SEQUENCE LISTING (20 sequences)

SEQ ID NO: 1 - protein, 1912 aa, synthetic construct [sequence omitted]

SEQ ID NO: 2 - protein, 1245 aa, synthetic construct
ETPPRFTRTPVDQTGVSGGVASFICQATGDPRPKIVWNKKGKKVSNQRFEVIEFDDGSGSVLRIQPLRTPRDEAIYECVASNNVGEISVSTRLTVLREDQIPRGFPTIDMGPQLKVVERTRTATMLCAASGNPDPEITWFKDFLPVDTSNNNGRIKQLRSESIGGTPIRGALQIEQSEESDQGKYECVATNSAGTRYSAPANLYVRELREVRRVPPRFSIPPTNHEIMPGGSVNITCVAVGSPMPYVKWMLGAEDLTPEDDMPIGRNVLELNDVRQSANYTCVAMSTLGVIEAIAQITVKALPKPPGTPVVTESTATSITLTWDSGNPEPVSYYIIQHKPKNSEELYKEIDGVATTRYSVAGLSPYSDYEFRVVAVNNIGRGPPSEPVLTQTSEQAPSSAPRDVQARMLSSTTILVQWKEPEEPNGQIQGYRVYYTMDPTQHVNNWMKHNVADSQITTIGNLVPQKTYSVKVLAFTSIGDGPLSSDIQVITQTGVPGQPLNFKAEPESETSILLSWTPPRSDTIANYELVYKDGEHGEEQRITIEPGTSYRLQGLKPNSLYYFRLAARSPQGLGASTAEISARTMQSKPSAPPQDISCTSPSSTSILVSWQPPPVEKQNGIITEYSIKYTAVDGEDDKPHEILGIPSDTTKYLLEQLEKWTEYRITVTAHTDVGPGPESLSVLIRTNEDVPSGPPRKVEVEAVNSTSVKVSWRSPVPNKQHGQIRGYQVHYVRMENGEPKGQPMLKDVMLADAQWEFDDTTEHDMIISGLQPETSYSLTVTAYTTKGDGARSKPKLVSTTGAVPGKPRLVINHTQMNTALIQWHPPVDTFGPLQGYRLKFGRKDMEPLTTLEFSEKEDHFTATDIHKGASYVFRLSARNKVGFGEEMVKEISIPEEVPTGFPQNLHSEGTTSTSVQLSWQPPVLAERNGIITKYTLLYRDINIPLLPMEQLIVPADTTMTLTGLKPDTTYDVKVRAHTSKGPGPYSPSVQFRTLPVDQVFAKNFHVKAVMKTSVLLSWEIPENYNSAMPFKILYDDGKMVEEVDGRATQKLIVNLKPEKSYSFVLTNRGNSAGGLQHRVTAKTAPDVLRTKPAFIGKTNLDGMITVQLPEVPANENIKGYYIIIVPLKKSRGKFIKPWESPDEMELDELLKEISRKRRSIRYGREVELKPYIAAHFDVLPTEFTLGDDKHYGGFTNKQLQSGQEYVFFVLAVMEHAESKMYATSPYSDPVVSMDLDPQPITDEEE

SEQ ID NO: 3 - protein, 297 aa, synthetic construct
PRFTRTPVDQTGVSGGVASFICQATGDPRPKIVWNKKGKKVSNQRFEVIEFDDGSGSVLRIQPLRTPRDEAIYECVASNNVGEISVSTRLTVLREDQIPRGFPTIDMGPQLKVVERTRTATMLCAASGNPDPEITWFKDFLPVDTSNNNGRIKQLRSESIGGTPIRGALQIEQSEESDQGKYECVATNSAGTRYSAPANLYVRELREVRRVPPRFSIPPTNHEIMPGGSVNITCVAVGSPMPYVKWMLGAEDLTPEDDMPIGRNVLELNDVRQSANYTCVAMSTLGVIEAIAQITVK

SEQ ID NO: 4 - protein, 82 aa, synthetic construct
SIPPTNHEIMPGGSVNITCVAVGSPMPYVKWMLGAEDLTPEDDMPIGRNVLELNDVRQSANYTCVAMSTLGVIEAIAQITVK

SEQ ID NO: 5 - protein, 30 aa, synthetic construct
HAEGTFTSDVSSYLEGQAAKEFIAWLVKGR

SEQ ID NO: 6 - protein, 30 aa, synthetic construct; identical to SEQ ID NO: 5
  MOD_RES 30: note = Amidation

SEQ ID NO: 7 - protein, 4 aa, synthetic construct
GSSG

SEQ ID NO: 8 - protein, 4 aa, synthetic construct
SGGS

SEQ ID NO: 9 - protein, 4 aa, synthetic construct
SGGS

SEQ ID NO: 10 - protein, 5 aa, synthetic construct
GGGGS

SEQ ID NO: 11 - protein, 10 aa, synthetic construct
SGGSGGSGGS

SEQ ID NO: 12 - protein, 16 aa, synthetic construct
SGSETPGTSESATPES

SEQ ID NO: 13 - protein, 32 aa, synthetic construct
SGGSSGGSSGSETPGTSESATPESSGGSSGGS

SEQ ID NO: 14 - protein, 9 aa, synthetic construct
HAHGHGHAH

SEQ ID NO: 15 - protein, 4 aa, synthetic construct
IEGR

SEQ ID NO: 16 - protein, 5 aa, synthetic construct
GGGRR

SEQ ID NO: 17 - protein, 1271 aa, synthetic construct
MYRMQLLSCIALSLALVTNSETPPRFTRTPVDQTGVSGGVASFICQATGDPRPKIVWNKKGKKVSNQRFEVIEFDDGSGSVLRIQPLRTPRDEAIYECVASNNVGEISVSTRLTVLREDQIPRGFPTIDMGPQLKVVERTRTATMLCAASGNPDPEITWFKDFLPVDTSNNNGRIKQLRSESIGGTPIRGALQIEQSEESDQGKYECVATNSAGTRYSAPANLYVRELREVRRVPPRFSIPPTNHEIMPGGSVNITCVAVGSPMPYVKWMLGAEDLTPEDDMPIGRNVLELNDVRQSANYTCVAMSTLGVIEAIAQITVKALPKPPGTPVVTESTATSITLTWDSGNPEPVSYYIIQHKPKNSEELYKEIDGVATTRYSVAGLSPYSDYEFRVVAVNNIGRGPPSEPVLTQTSEQAPSSAPRDVQARMLSSTTILVQWKEPEEPNGQIQGYRVYYTMDPTQHVNNWMKHNVADSQITTIGNLVPQKTYSVKVLAFTSIGDGPLSSDIQVITQTGVPGQPLNFKAEPESETSILLSWTPPRSDTIANYELVYKDGEHGEEQRITIEPGTSYRLQGLKPNSLYYFRLAARSPQGLGASTAEISARTMQSKPSAPPQDISCTSPSSTSILVSWQPPPVEKQNGIITEYSIKYTAVDGEDDKPHEILGIPSDTTKYLLEQLEKWTEYRITVTAHTDVGPGPESLSVLIRTNEDVPSGPPRKVEVEAVNSTSVKVSWRSPVPNKQHGQIRGYQVHYVRMENGEPKGQPMLKDVMLADAQWEFDDTTEHDMIISGLQPETSYSLTVTAYTTKGDGARSKPKLVSTTGAVPGKPRLVINHTQMNTALIQWHPPVDTFGPLQGYRLKFGRKDMEPLTTLEFSEKEDHFTATDIHKGASYVFRLSARNKVGFGEEMVKEISIPEEVPTGFPQNLHSEGTTSTSVQLSWQPPVLAERNGIITKYTLLYRDINIPLLPMEQLIVPADTTMTLTGLKPDTTYDVKVRAHTSKGPGPYSPSVQFRTLPVDQVFAKNFHVKAVMKTSVLLSWEIPENYNSAMPFKILYDDGKMVEEVDGRATQKLIVNLKPEKSYSFVLTNRGNSAGGLQHRVTAKTAPDVLRTKPAFIGKTNLDGMITVQLPEVPANENIKGYYIIIVPLKKSRGKFIKPWESPDEMELDELLKEISRKRRSIRYGREVELKPYIAAHFDVLPTEFTLGDDKHYGGFTNKQLQSGQEYVFFVLAVMEHAESKMYATSPYSDPVVSMDLDPQPITDEEEHHHHHH

SEQ ID NO: 18 - DNA, 3816 bp, synthetic construct
  source 1..3816: mol_type = other DNA
atgtacaggatgcaactcctgtcttgcattgcactaagtcttgcacttgtcacgaactcggagacacctccaaggtttacacgaacacccgttgatcagacaggggtctctggcggagttgcctctttcatctgccaagctacgggagacccaagacctaaaattgtctggaacaaaaaaggaaagaaagtcagcaatcagagatttgaggtaatagagtttgacgatgggtctggatcagttctcagaatacaacccttacggactccgagggatgaggccatttatgaatgtgtggcctcaaataatgtgggagaaataagtgtatccaccagactcacagttttgcgggaagatcaaattcccaggggcttccctaccattgacatgggcccacagttgaaggtggttgagcgtactcgcacggccaccatgctttgtgcagccagtggtaatccggatccagaaatcacttggtttaaagatttcttacctgtggacacaagcaacaacaatggtcgtattaagcagttacgatcagaatctattggtggtacaccaataagaggagcccttcagattgagcagagtgaagagtctgaccaaggaaaatatgagtgtgttgccaccaacagcgcgggcactcgctattccgctcctgccaatttatatgtcagagagctgcgagaagttcgccgtgtcccaccaagattctctatcccacccactaatcatgaaatcatgccaggcggaagcgttaatatcacctgtgtggccgtggggtcaccaatgccttatgtaaagtggatgttgggggcagaagatctgacacctgaagatgatatgccaataggaagaaatgtgctagaactgaatgatgtaagacagtcagcaaattacacctgtgttgctatgtcaacactgggtgtcattgaagcaatagcacagatcactgtcaaagccttacccaaacctccaggaactcctgtagtgaccgagagcacagctacaagcatcacactgacgtgggactctgggaaccctgagcctgtttcttattacataattcagcataaacctaaaaactctgaggaactttacaaagaaattgatggggtggcgaccacacgctacagtgtcgctggactaagtccctactcggattatgaattcagggttgttgctgtcaataacattgggcgggggcctcccagcgaacctgtgctaacacaaacctcagagcaagcaccatccagtgccccgagggatgtccaggcacgaatgttgagttcgaccaccattttggtacagtggaaggaacctgaagagccaaatggacagatccaaggatatagagtttattatacaatggatcccactcaacatgtcaacaactggatgaaacacaatgtagctgacagccaaatcactactattggcaacttagtgccccagaaaacatattctgtcaaagtcctggcttttacctcaattggagatggtcccctttcaagtgacatacaagtcatcactcagacaggagtaccagggcagccactaaacttcaaagcagaacctgagtctgaaacaagtattttgctctcttggacacctccacgttcagataccattgccaactatgaactggtctacaaagatggggagcatggagaggagcaacgaattaccattgagccagggacatcatataggctgcaaggactgaaaccaaacagcttatactatttccgtctggctgcacgctcccctcaaggcctgggtgcttctactgcagaaatatcagctagaaccatgcagtcaaagccgtcagctcctcctcaagacattagttgcaccagcccaagttccactagtattttggtaagttggcaacctccaccagtggaaaaacagaatggcattatcactgaatactccatcaagtacactgcagtggatggggaagatgacaagcctcacgagattttgggaattccttcggacactaccaaataccttttggaacagctggaaaaatggactgaataccggatcactgtgacagcccatacagatgtcggccctggccctgagagcttgtccgtgttgattcgaaccaatgaagatgttcctagtggtcctcctcgcaaagtcgaggtagaggctgtcaactcaacatctgttaaagtctcatggcgctcacccgtgcccaataaacagcatggccagataagaggatatcaggtgcattatgtgaggatggaaaatggtgagcccaagggccagcccatgctgaaagatgtcatgctggctgatgcacagtgggaatttgatgatactactgaacatgacatgatcatttctgggctccagcctgaaacttcctactccctcaccgtcacagcctacacaaccaaaggagatggtgctcgcagcaagcccaaactggtgtccaccactggggcagttccagggaaacctcggcttgtgattaaccacactcagatgaatactgctcttattcagtggcaccctccggtggacacatttggacctcttcagggctaccgtctaaaatttggccgcaaggatatggagccacttactactcttgagttctctgaaaaagaagatcactttacagctacagacatccacaagggagcatcatacgtcttcaggctctcagccagaaacaaagtgggctttggggaggagatggtgaaggagatttccattccagaagaagtaccaactggattccctcaaaaccttcactcagaaggcaccacttcaacctccgtccagttatcttggcaaccacctgtcctggcagagagaaatggcattatcaccaagtatacccttctttatagggatatcaacatcccccttctcccgatggagcagcttattgttccagctgacaccactatgacactcactggcttaaaaccagataccacatacgatgtaaaagtacgtgctcatacgagcaaagggcccgggccatatagtcccagtgtccagttcaggacactgcctgtggatcaagtgtttgcaaaaaattttcatgtcaaagcagtaatgaagacttccgtgttgctgtcttgggagattccagagaattataactccgccatgcctttcaaaattctttatgatgatgggaaaatggtagaagaagtggatggccgagccacacagaagttaattgtcaacctgaagcctgagaaatcatattcatttgtgctgacaaatcgtggaaacagtgctggtgggctgcagcacagggtcacggcaaagactgcaccagatgtattacgtaccaagcctgccttcattgggaagaccaacttggatggcatgattactgtgcaactgcctgaagtacctgcaaatgagaatataaaaggttactacataataattgtgcctttgaagaaatctcgcgggaaatttatcaagccatgggagagtccagatgaaatggaattagatgagctgcttaaggagatatctaggaagcgcagaagcatccgttatgggagagaagttgaattaaagccatatattgccgctcactttgatgtccttcccactgagttcaccctgggggatgacaagcattatggtggatttacaaacaagcaactccaaagtggtcaagaatatgtcttctttgtgttagcagtaatggaacatgcagagtctaagatgtatgcaaccagcccttactccgaccccgtggtgtcaatggatctggatccgcagccaatcacggatgaagaagaacaccaccaccatcaccactag

SEQ ID NO: 19 - protein, 92 aa, synthetic construct
PRFTRTPVDQTGVSGGVASFICQATGDPRPKIVWNKKGKKVSNQRFEVIEFDDGSGSVLRIQPLRTPRDEAIYECVASNNVGEISVSTRLTV

SEQ ID NO: 20 - protein, 100 aa, synthetic construct
TIDMGPQLKVVERTRTATMLCAASGNPDPEITWFKDFLPVDTSNNNGRIKQLRSESIGGTPIRGALQIEQSEESDQGKYECVATNSAGTRYSAPANLYVR

(19) United States
(12) Patent Application Publication
(10) Pub. No.: US 2024/0424057 A1
(43) Pub. Date: Dec. 26, 2024

(54) METHODS AND COMPOSITION OF INHIBITING ASPROSIN MEDIATED OREXIGENESIS AND/OR GLUCOGENSIS

(71) Applicants: CASE WESTERN RESERVE UNIVERSITY, Cleveland, OH (US); UNIVERSITY HOSPITALS CLEVELAND MEDICAL CENTER, Cleveland, OH (US)

(72) Inventors: Atul Chopra, Cleveland, OH (US); Ila Mishra, Cleveland, OH (US)

(21) Appl. No.: 18/685,081

(22) PCT Filed: Aug. 18, 2022
(86) PCT No.: PCT/US2022/075127

§ 371 Date: Feb. 20, 2024

Related U.S. Application Data
(60) Provisional application No. 63/234,505, filed on Aug. 18, 2021.

(51) Int. Cl. A61K 38/17; A61K 38/20; A61K 38/26; A61K 38/38; A61P 3/10

(52) U.S. Cl. CPC A61K 38/177; A61K 38/2013; A61K 38/26; A61K 38/38; A61P 3/10

(57) ABSTRACT

A method of inhibiting asprosin mediated orexigenesis and/or glucogenesis in a subject in need thereof includes administering to the subject a therapeutic peptide that includes an amino acid sequence substantially identical to an extracellular portion of the amino acid sequence of protein tyrosine phosphatase receptor type δ (PTPRD) that binds to asprosin.

### RELATED APPLICATION

This application claims priority from U.S. Provisional Application No. 63/234,505, filed Aug. 18, 2021, the subject matter of which is incorporated herein by reference in its entirety.

### GOVERNMENT FUNDING

This invention was made with government support under DK102529, awarded by The National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Aug. 18, 2022, is named CWR-030382WO ORD st.26 and is 27,594 bytes in size.

### BACKGROUND

Asprosin is a fasting-induced, glucogenic and orexigenic hormone. In cell-autonomous actions at the hepatocyte and hypothalamic AgRP neurons, asprosin induces hepatic glucose release into the circulation and appetite stimulation, respectively. Plasma asprosin crosses the blood-brain-barrier, and asprosin levels in the cerebrospinal fluid (CSF) increase when fasted, as they do in plasma. Identification of asprosin was made possible through study of a human genetic disease called Neonatal Progeroid syndrome (NPS), also known as Marfan Lipodystrophy syndrome. NPS patients display plasma asprosin deficiency, marked leanness, hypophagia and robust insulin sensitivity compared with age and sex matched control subjects. Mice (and rabbits) engineered to harbor NPS mutations (Fbnl^{NPS/+}) mimic the plasma asprosin deficiency, hypophagia, leanness and insulin sensitivity displayed by NPS patients. Moreover, Fbnl^{NPS/+} mice display remarkable resistance to metabolic syndrome despite being subjected to severe obesogenic and diabetogenic stress. Depressed AgRP⁺ neuron activity and low appetite displayed by NPS mice can be completely corrected upon replenishing plasma asprosin, thereby demonstrating a causal link. Asprosin was found elevated in metabolic syndrome patients (and rodent models) in a multitude of studies.

### SUMMARY

Embodiments described herein relate to compositions and methods of inhibiting asprosin mediated orexigenesis and/or glucogenesis, including orexigenic and/or glucogenic activity or function of asprosin in a subject in need thereof, and particularly relates to compositions and methods of treating or preventing one or more of insulin resistance, obesity, an obesity related condition, diabetes, a metabolic disease or disorder, such as metabolic syndrome, and being overweight in a subject in need thereof. We identified Protein Tyrosine Phosphatase Receptor δ (Ptprd), a membrane bound phosphatase receptor as the orexigenic and/or glucogenic asprosin-receptor. In mice, genetic ablation of Ptprd recapitulates the hypophagic and markedly lean phenotype associated with genetic asprosin deficiency and renders null mice unresponsive to the orexigenic effects of asprosin. AgRP⁺ neuron-specific Ptprd ablation renders neurons unresponsive to asprosin-mediated activation and leads to protection of mice from diet-induced obesity. As illustrated schematically in FIG. 1, we also found that a peptide comprising a portion of the asprosin ligand binding domain of Ptprd (i.e., Ptprd-ligand binding domain or Ptprd-lbd), when introduced ectopically into or administered in the circulation of obese mice, binds to and sequesters plasma asprosin leading to improvements in appetite, body weight, and blood glucose.

Accordingly, in some embodiments, a method of inhibiting asprosin mediated orexogenesis and/or glucogenesis in a subject in need thereof includes administering to the subject a therapeutically effective amount of an agent that inhibits asprosin mediated Ptprd signaling or activity. In some embodiments, the agent can include a therapeutic peptide that is a peptide mimetic of the asprosin ligand binding domain of Ptprd (i.e., Ptprd-lbd). The therapeutic peptide or peptide mimetic of the asprosin ligand binding domain of Ptprd can have an amino acid sequence substantially identical to an extracellular portion of the amino acid sequence of Ptprd that binds to asprosin.

In some embodiments, the therapeutic peptide can have an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to at least about 10, at least about 20, at least about 30, at least about 40, at least about 50, at least about 60, at least about 70, at least about 80, at least about 90, at least about 100, at least about 150, at least about 200, at least about 300, at least about 400, at least about 500, at least about 600, at least about 700, at least about 800, at least about 900, or at least 1000 consecutive amino acids of SEQ ID NO: 2.

In other embodiments, the therapeutic peptide has an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 2.

In still other embodiments, the therapeutic peptide can have an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to at least about 10, at least about 20, at least about 30, at least about 40, at least about 50, at least about 60, at least about 70, at least about 80, at least about 90, at least about 100, at least about 150, at least about 200 consecutive amino acids of SEQ ID NO: 3.

In some embodiments, the therapeutic peptide has a binding affinity K_{D} to asprosin less than about 10 μM, less than about 1 μM, less than about 500 nM, less than about 400 nM, less than about 300 nM, less than about 200 nM, less than about 100 nM, less than about 10 nM, less about 1 nM, or less than about 500 pM.

In some embodiments, the therapeutic peptide can be linked to at least one heterologous polypeptide. The at least one heterologous polypeptide can include, for example, an antibody or antigen binding fragment thereof, a glucagon-like peptide-1 receptor (GLP-1R) agonist, a Fc portion of an immunoglobulin, an albumin peptide, an albumin binding domain (ABD), a signal peptide, or a combination thereof.

In some embodiments, the heterologous polypeptide can include a glucagon-like peptide-1 receptor (GLP-1R) agonist. The GLP-IR agonist can include a bioactive GLP-1, a GLP-1 analogue, or a GLP-1 substitute. For example, the GLP-IR agonist can be selected from GLP-1 (7-37), GLP-1 (7-36) amide, exendin-4, liraglutide, CJC-1131, albugon, albiglutide, exantide, exenatide-LAR, oxyntomodulin, lixisenatide, geniproside, or a peptide fragment thereof with GLP-IR agonistic activity.

In other embodiments, the heterologous polypeptide can be linked to the therapeutic peptide with a peptide linker. In some embodiments, the linker can include a cleavable site that is cleavable by, for example, a protease, upon administration of the therapeutic peptide in vivo. For example, the peptide linker can include a protease cleavage site, such as IEGR (SEQ ID NO: 15) or GGGRR (SEQ ID NO: 16).

In some embodiments, the subject treated with the therapeutic peptide can have a body mass index (BMI) of 30 or greater.

In some embodiments, the therapeutic peptide is administered to the subject by parenteral administration of a fusion polypeptide comprising the therapeutic peptide.

In other embodiments, the therapeutic peptide can be administered to subject by expressing the therapeutic peptide from cells of the subject.

In some embodiments, the cells can be transfected with a vector encoding the therapeutic peptide.

In some embodiments, the expressed therapeutic peptide can be linked to a signal peptide sequence that promotes secretion of the expressed therapeutic peptide from the cells.

In other embodiments, the vector can include a nucleic sequence having SEQ ID NO: 18.

In some embodiments, the vector is a recombinant adeno-associated virus (AAV) vector having a capsid selected from AAV1, AAV2, AAV6, AAV8, AAV9, AAVrh74, AAVrh10, AAV5, AAV7, AAVS3, AAVHSC, AAV2.7m8, AAV-LK03, AAV8/Olig001, AAV218, AAVhu37, AAV2tYF, AAVh1, AAVhu68, AAVrh.8, AAVrh9, AAV.PHP.B., AAV.PHP.eB, AAV.PHP.S, AAV/BBB, AAV-DJ, AAVr3.45, AAV-sh10, AAV2 (Y444F), AAV4, AAV-RPF2, AAV3b, AAVrh64R1, or variants thereof.

Other embodiments described herein relate to a fusion polypeptide or protein that includes a therapeutic peptide having an amino acid sequence substantially identical to an extracellular portion of the amino acid sequence of protein tyrosine phosphatase receptor type δ (Ptprd) that binds to asprosin and at least one polypeptide having an amino acid sequence heterologous to the therapeutic peptide.

In some embodiments, the therapeutic peptide of the fusion polypeptide can have an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to at least about 10, at least about 20, at least about 30, at least about 40, at least about 50, at least about 60, at least about 70, at least about 80, at least about 90, at least about 100, at least about 150,at least about 200, at least about 300, at least about 400, at least about 500, at least about 600,at least about 700, at least about 800, at least about 900, or at least 1000 consecutive amino acids of SEQ ID NO: 2.

In other embodiments, the therapeutic peptide of the fusion polypeptide can have an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 2.

In still other embodiments, the therapeutic peptide of the fusion polypeptide can have an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to at least about 10, at least about 20, at least about 30, at least about 40, at least about 50, at least about 60, at least about 70, at least about 80, at least about 90, at least about 100, at least about 150,at least about 200 consecutive amino acids of SEQ ID NO: 3.

In some embodiments, the therapeutic peptide can have a binding affinity K_{D} to asprosin less than about 10 μM, less than about 1 μM, less than about 500 nM, less than about 400 nM, less than about 300 nM, less than about 200 nM, less than about 100 nM, less than about 10 nM, less about 1 nM, or less than about 500 pM.

In other embodiments, the at least one heterologous polypeptide of the fusion polypeptide can include an antibody or antigen binding fragment thereof, a glucagon-like peptide-1 receptor (GLP-1R) agonist, a Fc portion of an immunoglobulin, an albumin peptide, an albumin binding domain (ABD), a peptide linker, a signal peptide, or a combination thereof.

In some embodiments, the heterologous polypeptide of the fusion polypeptide can include a glucagon-like peptide-1 receptor (GLP-1R) agonist. The GLP-IR agonist can include a bioactive GLP-1, a GLP-1 analogue, or a GLP-1 substitute. For example, the GLP-1R agonist can be selected from GLP-1 (7-37), GLP-1 (7-36) amide, exendin-4, liraglutide, CJC-1131, albugon, albiglutide, exantide, exenatide-LAR, oxyntomodulin, lixisenatide, geniproside, or a peptide fragment thereof with GLP-IR agonistic activity.

In some embodiments, a peptide linker can link the therapeutic peptide to the GLP-1R agonist. the linker comprises a protease cleavable site. In some embodiments, the linker can include a cleavable site that is cleavable by, for example, a protease, upon administration of the therapeutic peptide to a subject. For example, the peptide linker can include a protease cleavage site, such as IEGR (SEQ ID NO: 15) or GGGRR (SEQ ID NO: 16).

In other embodiments, the fusion polypeptide can include a signal peptide that promotes secretion of the expressed therapeutic peptide from cells. For example, the signal peptide can be an IL-2 signal peptide that promotes secretion of the fusion polypeptide from cells.

In some embodiments, the fusion polypeptide can be formulated as a medicament for use in inhibiting asprosin mediated orexigenesis and/or glucogenesis in a subject in need thereof.

Other embodiments described herein relate to a viral vector that includes a nucleic acid encoding a fusion polypeptide or a therapeutic peptide as described herein.

In some embodiments, the viral vector can include expression control sequences that direct expression of the fusion polypeptide in host cells.

In some embodiments, the viral vector can include a nucleic sequence having SEQ ID NO:18.

In some embodiments, the vector is a recombinant adeno-associated virus (AAV) vector having a capsid selected from AAV1, AAV2, AAV6, AAV8, AAV9, AAVrh74, AAVrh10, AAV5, AAV7, AAVS3, AAVHSC, AAV2.7m8, AAV-LK03, AAV8/Olig001, AAV218, AAVhu37, AAV2tYF, AAVh1, AAVhu68, AAVrh.8, AAVrh9, AAV.PHP.B., AAV.PHP.eB, AAV.PHP.S, AAV/BBB, AAV-DJ, AAVr3.45, AAV-sh10, AAV2 (Y444F), AAV4, AAV-RPF2, AAV3b, AAVrh64R1, or variants thereof.

Still other embodiments described herein related to a pharmaceutical composition that includes a pharmaceutically acceptable carrier or excipient and a fusion polypeptide or a viral vector as described herein. The pharmaceutical composition can be used for inhibiting asprosin mediated orexigenesis and/or glucogenesis in a subject in need thereof.

In some embodiments, the therapeutic peptide can bind to and sequester asprosin in circulation of the subject.

### BRIEF DESCRIPTION OF THE DRAWINGS

FIG. 1 illustrates a graphical representation of sequestering of asprosin with PTPRD-LBD.

FIGS. 2 (A-I) illustrate the identification of an asprosin-interacting receptor in the mouse brain. (A) Dual-label immunofluorescence RNAscope hybridization showing relative overlap between AgRP and Olfr734 expressing cells in the arcuate (ARC) nucleus of wild type (WT) mice. Scale bars, 100 μm. 3V, 3^{rd} ventricle. (B) qPCR results showing relative mRNA levels of Olfr734 (left) from visually enriched tdTOMATO expressing neurons of AgRP-cre mice and (right) cre-responsive ribotag enriched transcripts from AgRP neurons of AgRP-cre mice. (C) Schematic overview of asprosin immunoprecipitation (IP; left) of brain tissue incubated with recombinant asprosin or recombinant GFP before mass spectrometry (MS) analysis, (right) overlap of candidate asprosin-interacting proteins from 3 repeats of IP/MS. 58 candidate proteins, including Ptprd appeared in all 3 repeats. (D) Dual-label immunofluorescence RNAscope hybridization showing relative overlap between AgRP and Piprd expressing cells in the arcuate (ARC) nucleus of wild type (WT) mice. Scale bars, 100 ηm. 3V, 3^{rd} ventricle. (E) qPCR results showing relative mRNA levels of Ptprd (left) from visually enriched tdTOMATO expressing neurons of AgRP-cre mice and (right) cre-responsive ribotag enriched transcripts from AgRP neurons of AgRP-cre mice. (F) Reciprocal in vitro immunoprecipitation of recombinant GST-asprosin and extracellular domain of his-tagged PTPRD (PTPRD-6his), with mouse IgG and rabbit IgG as negative controls. (G-I) Quantification of the binding affinity between Asprosin and PTPRD by Microscale thermophoresis (k; MST; Kd of 4.2±10.2 nM), Bio-layer interferometry (1; BLI; Kd of 57.0±1.83 nM), and by Surface plasmon resonance analysis (m; SPR; Kd of 36.8±5.7 nM). Data are represented as mean±SEM.

FIGS. 3(A-R) illustrate Ptprd ablation phenocopies the body weight and orexigenic deficits associated with genetic asprosin deficiency and pharmacologic asprosin inhibition. (A) Representative photograph of 5-months-old male Ptprd^{+/+} and Ptprd^{-/-} littermates. (B) Weekly body weights of wildtype (Ptprd^{+/+}, n=25) and Piprd null (Ptprd^{-/-}, n=11) male mice from 5-to 12-weeks of age. (C-D) Body composition data using Magnetic Resonance Imaging (MRI) on 5-month-old Ptprd^{+/+} and Ptprd^{-/-} male mice on normal chow (n=4/group). (E-G) Cumulative food intake (e), total food intake in the dark and light phase (f), and ANCOVA analysis (g) of total food intake of Ptprd^{+/+} and Ptprd^{-/-} male mice measured over 3 days using the Promethion metabolic system (n=12 or 13/group). (H-J) Hourly energy expenditure (h) total energy expenditure in the light and dark phase (i), and ANCOVA analysis (j) of energy expenditure of Ptprd^{+/+} and Ptprd^{-/-} male mice measured over 3 days using the Promethion metabolic system (n=13/group). (K) Representative photograph of 6-month-old female Ptprd^{+/+} and Ptprd^{-/-} littermates. (L) Weekly body weights of wildtype (Ptprd^{+/+} n=16) and Ptprd null (Ptprd^{-/-}, n=11) female mice from 5- to 10-weeks of age. (M-O) Cumulative food intake (m), total food intake in the dark and light phase (n), and ANCOVA analysis (o) of total food intake of Ptprd^{+/+} (n=8) and Ptprd^{-/-} (n=9) female mice measured over 3 days using Promethion metabolic system. (P-R) Hourly energy expenditure (P) and total energy expenditure in the light and dark phase (Q), and ANCOVA analysis (R) of energy expenditure of Ptprd^{+/+} (n=8) and Ptprd^{-/-} (n=9) female mice measured over 3 days using the Promethion metabolic system. *P<0.05, **P<0.01, ***P<0.001 and ****P<0.0001; effect of genotype' determined by two-tailed t-test (C, D, F, I, N, Q), Repeated measures two-way ANOVA (B, E, H, L, M, P) or ANCOVA (G, J, O, R). Data are represented as mean±SEM. See also FIGS. 9, 10, 11 and 12.

FIGS. 4(A-O) illustrate whole body loss of a single allele and AgRP neuron-specific complete loss of Ptprd is protective against diet induced obesity in females. (A) Weekly body weights of wildtype (Ptprd^{+/+}; n=16) and heterozygous Ptprd knockout (Ptprd^{+/-}; n=24) female mice maintained on ad libitum normal chow (NC) diet. (B, C) Cumulative food intake and hourly energy expenditure of 10-week-old Ptprd^{+/+} and Ptprd^{+/-} female mice on NC over 3 days using the Promethion metabolic system (food intake: Ptprd^{+/+} n=8; Piprd^{+/-} n=10; Energy expenditure: Ptprd^{+/+} n=8; Ptprd^{+/-} n=14). (D, E) Weekly body weight change of Ptprd^{+/+} and Ptprd^{+/-} females maintained on high fat diet (HFD) from 5-weeks of age, with food intake measured in a randomly selected cohort of 16-week-old female mice from each group (body weight change: Ptprd^{+/+} n=9, Ptprd^{+/-} n=12; food intake: Ptprd^{+/+} n=4; Ptprd^{+/-} n=4). (F) Body weight of 6-week-old Ptprd^{flox/flox} and AgRP-cre; Ptprd^{flox/flox} maintained on ad libitum NC diet (Ptprd^{flox/flox} n=9, AgRP-cre; Ptprd^{flox/flox} n=10) (G, H) Cumulative food intake and hourly energy expenditure of 8-week-old Ptprd^{flox/flox} and AgRP-cre; Ptprd^{flox/flox} mice maintained on ad libitum NC over 4 days using the Promethion system (n=4/group). (I, J) Weekly body weight change of Ptprd^{flox/flox} (n=9), AgRP-cre; Ptprd^{flox/+} (n=4) and AgRP-cre; Ptprd^{flox/flox} (n=10) females maintained on high fat diet (HFD) from 5 weeks of age, with food intake measured in 14-week-old females from each group (Ptprd^{flox/flox}: n=8, AgRP-cre; Ptprd^{flox/+}: n=5 and AgRP-cre; Ptprd^{flox/flox}: n=8). (K) schematic showing bilateral stereotactic injection of virus (AAV) containing Ptprd sgRNA with AAV expressing mCherry (control) or Cas9 (AgRP^{Ptprd-KO}) in the arcuate (ARC) nucleus of adult AgRP-cre female mice. (L) Body weight of control and AgRP^{Ptprd-KO} female mice maintained on NC diet from day 0 to day 48 post stereotactic injection (control: n=8; AgRP^{Ptprd-KO}: n=11). (M) Cumulative food intake measured every fourth day of control and AgRP^{Ptprd-KO} female mice maintained on NC diet (control: n=6; AgRP^{Ptprd-KO}: n=11). (N, O) Body weight change and cumulative food intake of control and AgRP^{Ptprd-KO} female mice subjected to HFD on day 48 post stereotactic injection of AAVs coding cas9 and Ptprd sgRNA (control: n=6-7; AgRP^{Ptprd-KO}: n=11). *P<0.05, **P<0.01, ***P<0.001 and ****P<0.0001; by student T-test (E, F, J, L), ‘effect of genotype’ determined by repeated measures two-way ANOVA (A, B, C, G, H) with point-comparison by multiple unpaired T-test (D, I, M, N, O). Circle (O) and square (D) symbol represents mice maintained on NC diet and HFD, respectively. Data are represented as mean±SEM.

FIGS. 5(A-G) illustrates the deletion of Ptprd renders AgRP neurons unresponsive to asprosin. (A) Schematic showing unilateral stereotactic injection of virus (AAV) containing Ptprd sgRNA with AAV expressing mCherry (control) or Cas9 (AgRP^{Ptprd-KO}) in the arcuate (ARC) nucleus of adult AgRP-cre male mice (B-D) Representative action potential firing traces, baseline neuronal firing rate and membrane potential of AgRP neurons from AgRP-cre mice subjected to overnight fasting or ad libitum feeding (control+fed: n=28; control+fasted: n=31; Ptprd KO+fed: n=31; Ptprd KO+fasted: n=28) after unilateral stereotactic injection of Ptprd sgRNA+mCherry expressing AAVs on one side (control side), and Ptprd sgRNA+Cas9 expressing AAVs in the other side (AgRP^{Ptprd-KO}; KO side) ARC of hypothalamus (E-G) Representative action potential firing traces, neuronal firing rate and membrane potential of AgRP neurons from AgRP-Cre mice, incubated with recombinant GFP or asprosin for 2 hours (Control+GFP: n=13, Control+Asprosin: n=13, Ptprd KO+GFP: n=13, Ptprd KO+Asprosin: n=11) after unilateral stereotactic injection of Ptprd sgRNA+mCherry expressing AAVs on one side (control side), and Piprd sgRNA+Cas9 expressing AAVs in the other side (AgRP^{Ptprd-KO}; KO side) of ARC of the hypothalamus. *P<0.05, **P<0.01, ***P<0.001 and ****P<0.0001; by two-tailed T-test. Data are represented as mean±SEM.

FIGS. 6(A-F) illustrate Ptprd^{-/-} mice are unresponsive to the orexigenic effects of asprosin while responding normally to its glucogenic effects. (A-C) 14-week-old normal chow fed Ptprd^{+/+} and Ptprd^{-/-} male mice tail-vein transduced with Ad-empty or Ad-hFBNI (3.6×10⁹ pfu/mouse) viruses. (A) Cumulative food intake measured in Promethion metabolic chamber over 3 days; day 8 to day 11 post adenoviral vector transduction (Ad-empty: n=8 Ptprd^{+/+} and n=10 Ptprd^{-/-}; Ad-hFBNl: n=7 Ptprd^{+/+} and n=6 Ptprd^{-/-}. (B, C) ad libitum fed baseline blood glucose levels and glucose tolerance assay in response to intraperitoneal glucose injection (2 mg/kg) on day 14 post adenoviral transduction (baseline glucose: Ad-empty: n=5 Ptprd^{+/'} and n=6 Ptprd^{-/-}; Ad-hFBNl: n=8 Ptprd^{+/+} and n=6 Ptprd^{-/-}, GTT: Ad-empty: n=5 Ptprd^{+/30} and n=4 Ptprd^{-/-}; Ad-hFBNl: n=7 Ptprd^{+/+} and n=4 Ptprd^{-/-}). (D-F) 12-week-old normal chow fed Ptprd^{+/+} and Ptprd^{-/-} male mice tail-vein transduced with Ad-empty or Ad-hAsprosin (5×10¹⁰ pfu/mouse) viruses. (D) Cumulative food intake measured in promethion metabolic chamber over 4 days; day 8 to day 12 post adenoviral vector transduction (Ad-empty: n=8 Ptprd^{+/+} and n=8 Ptprd^{-/-}; Ad-hAsprosin: n=9 Ptprd^{+/+} and n=6 Ptprd^{-/-}. (E,F) ad libitum fed baseline blood glucose levels and glucose tolerance assay (GTT) in response to intraperitoneal glucose injection (2 mg/kg) on day 15 post adenoviral transduction (Ad-empty: n=7 Ptprd^{+/+} and n=10 Ptprd^{-/-}; Ad-hAsprosinl: n=7 Ptprd^{+/+} and n=7 Ptprd^{-/-}). *P<0.05, **P<0.01, ***P<0.001 and ****P<0.0001; by two-tailed t-test (A, B, D, E), and repeated measures two-way ANOVA (C, F). Data are represented as mean±SEM.

FIGS. 7(A-N) illustrate asprosin-mediated Ptprd signaling leads to Stat3 dephosphorylation and inactivation. (A) phosphorylated-Stat3 (p-Stat3) levels measured using ELISA in hypothalamic neural lysate of wild-type normal chow fed lean mice, 15 days post tail-vein transduction with Ad-empty or Ad-hFBNl (3.6×10⁹ pfu/mouse; n=7 or 8/group) viruses. (B) p-Stat levels measured using ELISA in hypothalamic neural lysate of normal chow fed lean Ptprd^{+/+} and Ptprd^{-/-} male mice (n=7 or 8/group). (C-D) Western blot analysis of β-actin, p-Stat3 and Stat3 in hypothalamic neural lysate of normal chow fed Ptprd^{+/+} and Ptprd^{-/-} male mice (n=6/group). (E) PTPRD mRNA levels measured in HEK293T cells 24 h post transfection with scrambled (control) and Ptprd siRNA (25 nM; n=3/treatment). (F-G) Western blot analysis of beta actin, p-Stat3 and Stat3 in HEK293T cell lysate 48 h post transfection with control and PTPRD siRNA (25 nM; n=4/treatment). (H) Stat3-response element driven luciferase activity measured in HEK293T cells transduced with 2 μg 4×M67 pTATA-TK-Luc plasmid with control or PTPRD siRNA treatment (25 nM; n=11/treatment). (I,J) Validation of asprosin overexpression in HEK293T cells using IL2-his-asprosin expressing mammalian expression plasmid (empty plasmid as control;i) and Ad5-IL2-his-Asprosin (Ad5-empty as control; j) under Ptprd knockdown condition (non-targeted scrambled siRNA as control; n=4/group). (K) Percent change in Stat3-response element driven luciferase activity measured in HEK293T cells 72 hours post co-transfection of 2 μg 4×M67 pTATA-TK-Luc plasmid with serial dilution of empty or IL2-his-asprosin expressing mammalian expression plasmid (0.25, 0.5, 1 and 2 μg plasmid; n=5 or 6/treatment). (L) Stat3-response element driven luciferase activity measured in HEK293T cells 72 hours post co-transfection of 2 μg 4×M67 pTATA-TK-Luc plasmid with 1 μg empty or asprosin expressing plasmid under conditions of control or PTPRD knockdown (25 nM; n=9or 10/treatment). (M,N) Western blot analysis of β-actin, p-Stat3 and Stat3 of HEK293T cell lysate, 72 h post co-treatment of control and PTPRD siRNA (25 nM) with control or asprosin expressing Ad5 vectors (100 vp/cell; n=4/treatment). *P<0.05, **P<0.01, ***P<0.001 and ****P<0.0001; by two-tailed t-test. Data are represented as mean±SEM. See also FIG. 16.

FIGS. 8(A-Q) illustrate the introduction of the PTPRD ligand-binding-domain (PTPRD-LBD) into the circulation functions as an “asprosin-trap” to ameliorate appetite, body weight and blood glucose in obese mice. (A) Asprosin detected by sandwich ELISA in 5 nM recombinant asprosin (rAsprosin) preincubated with 500 ng GFP or recombinant PTPRD-ligand binding domain (rPTPRD-LBD) for one hour in phosphate buffer saline (n=4/treatment). Asprosin levels plotted relative to signal detected in rAsprosin+GFP. (B-C) Adenoviral vector (Ad5) mediated overexpression of the IL2-tagged PTPRD ligand-binding-domain (Ptprd-LBD) detected at mRNA level in hepatic tissue (B) and as protein in plasma (C) of 18-week-old DIO mice, 15 days post tail vein injection of Ad5-GFP or Ad5-hPTPRD-LBD (1×10¹¹ vp/mouse) viruses. (D) Plasma asprosin levels of 18-week-old DIO mice, 4 days post tail vein injection of Ad5-GFP or Ad5-hPTPRD-LBD (2×10¹¹ vp/mouse) viruses. Plasma asprosin plotted relative to day 0 asprosin levels for each mouse (n=11 Ad5-GFP and 10 Ad5-hPTPRD-LBD). (E-H) Food intake, body weight change, baseline glucose and glucose tolerance test (GTT) measured in 18-week-old male DIO mice 12-14 days post tail-vein-injection of Ad5-GFP or Ad5-hPTPRD-LBD (1×10¹¹ vp/mouse) viruses (body weight change, food intake: Ad5-GFP: n=9; Ad5-hPTPRD-LBD: n=10; baseline glucose: n=10/treatment; GTT: Ad5-GFP: n=8; Ad5-hPTPRD-LBD: n=7). (I-K) Representative action potential firing traces, depolarization and firing frequency of AgRP neurons after recombinant asprosin (rAsprosin), rAsprosin+recombinant PTPRD-LBD (rPTPRD-LBD), and rAsprosin treatment (n=6/treatment). (L-N) Representative action potential firing traces, depolarization and firing frequency of AgRP neurons after rAsprosin and rAsprosin+GFP treatment, to test the effect of irrelevant protein (GFP) treatment (n=5/treatment). (O,P) Western blot analysis of β-actin, p-Stat3 and Stat3 in 10 μg cell lysate from HEK293T cells transfected with empty-backbone (pEmpty) or asprosin coding (pAsprosin) mammalian expression plasmid (2 μg) for 48 hours, followed by 8 hours of treatment with rGFP or rPTPRD-LBD (1 μg; n=4/treatment). (Q) Percent change in Stat3-response element driven luciferase activity measured in HEK293T cells treated with GFP or rPTPRD-LBD (1 μg) for 12 hours, 72 hours post co-transfection of 2 μg 4×M67 pTATA-TK-Luc plasmid with 1 μg pEmpty or pAsprosin (pEmpty+GFP: n=11; pAsprosin+GFP: n=12; pAsprosin+rPTPRD-LBD: n=11). *P<0.05, **P<0.01, ***P<0.001 and ****P<0.0001; by two-tailed t-test and 2-way ANOVA (H). Data are represented as mean±SEM. See also FIG. 17.

FIG. 9 illustrates an analysis of covariance of energy expenditure in active and inactive phase. Analysis of covariance (ANCOVA) of energy expenditure measured during day and night period (12 h Light: 12 h Dark) in Ptprd^{+/+} and Ptprd^{-/-} male and female mice housed in Promethion metabolic chambers for 3 days under ad libitum normal chow feeding condition. For statistical significance, alpha (α) was set at 0.05.

FIGS. 10 (A-B) illustrate plasma lipid and metabolic hormone profile associated with Ptprd ablation. (A) Plasma levels of low-density lipoproteins (LDL), high-density lipoprotein (HDL), total cholesterol (TC), triglycerides (TG), free fatty acids (FFA), and glycerol measured in plasma of 14-week-old ad libitum fed Ptprd^{+/+}, Ptprd^{+/-}, Piprd^{-/-} male mice (n=9/group). (B) Plasma levels of key metabolic hormones, asprosin, adiponectin, ghrelin, and leptin, measured in plasma of 14-week-old ad libitum fed Ptprd^{+/+}, Ptprd^{+/-}, Ptprd^{-/-}male mice (n=9/group except plasma asprosin measured in n=12 Ptprd^{+/+}, n=12 Ptprd^{+/-} and n=15 Ptprd^{-/-} mice). Asterisk (*) indicate the range of alpha; *p<0.05, **p<0.01, ***p<0.001, and ****p<0.0001; by two-tailed T-test. Data are represented as mean±SEM.

FIGS. 11(A-D) illustrate locomotor activity and respiratory exchange ratio of Ptprd-null mice, cumulative locomotor activity. (A, C) and hourly respiratory exchange ratio (R.E.R.; B, D) measured in 12-week-old male and 10-week-old female Ptprd^{+/+} and Ptprd^{-/-} mice over 3 days in Promethion metabolic chambers under ad libitum normal chow feeding condition. For statistical significance, alpha (α) was set at 0.05 with repeated measures Two-way ANOVA. Data are represented as mean±SEM.

FIGS. 12(A-G) illustrate Ptprd is dispensable for the glucogenic function of Asprosin. (A) qPCR of Ptprd expression in various tissues of wild type male mice (n=5). (B-C) Blood glucose levels of Ad libitum fed and 18-hour-fasted 12-week-old male mice (n=17 Ptprd^{+/+}, 16 Ptprd^{+/-} and 12 Ptprd^{-/-}). (D) Glucose measured at 0, 15, 30, 60 and 120 min post intraperitoneal (IP) injection of 2 mg/kg glucose solution in 18-hour-fasted 12-week-old male mice (n=15 Ptprd^{+/+}, 16 Ptprd^{+/-} and 10 Ptprd^{-/-}). (E, F) Blood glucose levels of Ad libitum fed and 24-hour-fasted 10-week-old female mice (n=13 Ptprd^{+/+}, 14 Ptprd^{+/-} and 12 Ptprd^{-/-}). (G) Glucose measured at 0, 15, 30, 60 and 120 min post intraperitoneal injection of 2 mg/kg glucose solution in 24-hour-fasted female mice (n=13Ptprd^{+/+}, 14 Ptprd^{+/-} and 12 Ptprd^{-/-}). One-way (B, C, E, F) and two-way ANOVA (D, G) tested statistical significance with alpha (α) set at 0.05. Data are represented as mean±SEM.

FIGS. 13(A-D) illustrate energy expenditure, locomotor activity and respiratory exchange ratio of AgRP neuron-specific Ptprd knock-out female mice on a high fat diet. Body weight (A), cumulative locomotion activity (B), respiratory exchange ratio (C), and hourly energy expenditure of 14-week-old Ptprd^{flox/flox} (n=6), AgRP-cre; Ptprd^{flox/+} (n=4) and AgRP-cre; Ptprd^{flox/flox} (n=6) females maintained on high fat diet (HFD) from 5-weeks of age. Change in body weight and food intake presented in FIG. 3I, J. Asterisk (*) indicate the range of alpha; *p<0.05, **p<0.01, ***p<0.001, and ****p<0.0001, as determined by one-way ANOVA (a) and two-way ANOVA (B-D). Data are represented as mean±SEM.

FIGS. 14(A-O) illustrate whole body loss of a single allele and AgRP neuron-specific loss of Ptprd is not protective against diet induced obesity in males. (A) Weekly body weight of wildtype (Ptprd^{+/+}; n=24) and heterozygous (Ptprd^{+/-}; n=25) male mice maintained on ad libitum normal chow (NC) diet. (B, C) Cumulative food intake and hourly energy expenditure of 12-week-old Ptprd^{+/+} and Ptprd^{+/-} mice on NC over 3 days using the Promethion system (food intake: Ptprd^{+/+} n=14; Ptprd^{+/-} n=15; energy expenditure: Ptprd^{+/+} n=13; Ptprd^{+/-} n=13). (D, E) Weekly body weight change of Ptprd^{+/+} and Ptprd^{+/-} males maintained on high fat diet (HFD) from 5 weeks of age, with food intake measured in a randomly selected cohort of 16-week-old mice from each group (Body weight change: Ptprd^{+/+} n=9, Ptprd^{+/-} n=7; food intake: Ptprd^{+/+} n=5; Ptprd^{+/-} n=4). (F) Body weight of 8-week-old Ptprd^{flox/flox} and AgRP-cre; Ptprd^{flox/flox} males maintained on ad libitum NC diet (n=4/group) (G, H) Cumulative food intake and hourly energy expenditure of 8-week-old Ptprd^{flox/flox} and AgRP-cre; Ptprd^{flox/flox} male mice maintained on ad libitum NC over 4 days using the Promethion system (n=4/group). (I, J) Weekly body weight change of Ptprd^{flox/flox} (n=16), AgRP-cre; Ptprd^{flox/+} (n=9) and AgRP-cre; Ptprd^{flox/flox} (n=11) males maintained on high fat diet (HFD) from 5 weeks of age, with food intake measured in a randomly selected cohort of 14-week-old mice from each group (n=9/group). (K) Schematic showing bilateral stereotactic injection of virus (AAV) containing Ptprd sgRNA with AAV expressing mCherry (control) or Cas9 (AgRP^{Ptprd-KO}) in the arcuate (ARC) nucleus of adult AgRP-cre male mice. (L) Body weight of control and AgRP^{Ptprd-KO} male mice maintained on NC diet from day 0 to day 48 post stereotactic injection (control: n=9 and AgRP^{Ptprd-KO}: n=9). (M) Cumulative food intake measured every week of control and AgRP^{Ptprd-KO} male mice maintained on NC diet (control: n=9 and AgRP^{Ptprd-KO}: n=9). (N, O) Body weight change and cumulative food intake of control and AgRP^{Ptprd-KO} male mice subjected to HFD on day 48 post stereotactic injection of AAVs coding cas9 and Ptprd sgRNA (control: n=9 and AgRP^{Ptprd-KO}: n=9). *P<0.05, **P<0.01, ***P<0.001 and ****P<0.0001; by student T-test (E, F, J, L), ‘effect of genotype’ determined by repeated measures two-way ANOVA (A-D; G-I; M-O). Circle (O) and square symbol represents mice maintained on NC diet and HFD, respectively. Data are represented as mean±SEM.

FIGS. 15 (A-B) illustrate adenovirus mediated overexpression of asprosin. Human asprosin levels detected in plasma of Ad5-FBNl. (A) and Ad5-Asprosin (B) injected Ptprd^{+/+} and Ptprd^{-/-} male mice is plotted relative to the average background signal detected in Ad5-empty injected mice. Asterisk (*) indicate the range of alpha; *p<0.05, **p<0.01, ***p<0.001, and ****p<0.0001, as determined by two-tailed T-test. Data are represented as mean±SEM.

FIGS. 16 (A-B) illustrate asprosin neutralization rescues p-Stat3 response in diet-induced obese mice and HEK293T cells. (A) phosphorylated-Stat3 (p-Stat3) levels measured using ELISA in hypothalamic neural lysate diet-induced obese (DIO) male mice, 16 h post intraperitoneal treatment with anti-asprosin monoclonal antibody (mAb) or IgG control (500 μg in 500 ml saline/mouse; n=6/group). Note that mice were without food for the duration of the experiment, demonstrating that the effect on hypothalamic p-stat was independent of the mAb induced changes in food intake and adiposity. (B) Percent change in Stat3-response element driven luciferase activity measured in pTATA-TK-Luc plasmid transfected HEK293T cells, treated for 18 h with asprosin conditioned media preincubated with anti-asprosin mAb (or IgG control 500 ng/well), or 18 h ectopic treatment of anti-asprosin mAb (IgG control; 500 ng/well) 48 h post co-transfection of Empty or Asprosin expressing mammalian plasmid (n=10 or 11/group). *P<0.05, **P<0.01, ***P<0.001 and ****P<0.0001; by two-tailed t-test. Data are represented as mean±SEM.

FIG. 17 illustrates PTPRD ligand binding domain sandwich ELISA. A custom-built sandwich ELISA using rabbit anti-PTPRD (100 ng/well) as the capture antibody, and mouse anti-his (100 ng/well) as the detection antibody. An anti-mouse secondary antibody (1:5000) linked to HRP was used to generate a signal, and recombinant PTPRD was used to generate a standard curve.

FIG. 18 illustrate a nucleotide sequence (SEQ ID NO:18) of IL2-Ptprd extracellular domain (with C-terminal 6His tag)

FIG. 19 illustrates the amino acid sequence (SEQ ID NO:17) of IL2-Ptprd-extracellular domain with a C-terminal 6HIS tag.

### DETAILED DESCRIPTION

Unless otherwise defined, scientific and technical terms used herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art.

For convenience, certain terms employed in the specification, examples, and appended claims are collected here. Unless defined otherwise, all technical and scientific terms used herein have the same meaning as commonly understood by one of ordinary skill in the art to which this application belongs.

The articles “a” and “an” are used herein to refer to one or to more than one (i.e., to at least one) of the grammatical object of the article. By way of example, “an element” means one element or more than one element.

The terms “comprise,” “comprising,” “include,” “including,” “have,” and “having” are used in the inclusive, open sense, meaning that additional elements may be included. The terms “such as”, “e.g.,”, as used herein are non-limiting and are for illustrative purposes only. “Including” and “including but not limited to” are used interchangeably.

The term “or” as used herein should be understood to mean “and/or” unless the context clearly indicates otherwise.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

As used herein, “one or more of a, b, and c” means a, b, c, ab, ac, bc, or abc. The use of “or” herein is the inclusive or.

The term “administering” to a patient includes dispensing, delivering or applying an active compound in a pharmaceutical formulation to a subject by any suitable route for delivery of the active compound to the desired location in the subject (e.g., to thereby contact a desired cell), including administration into the cerebrospinal fluid or across the blood-brain barrier, delivery by either the parenteral or oral route, intramuscular injection, subcutaneous or intradermal injection, intravenous injection, buccal administration, transdermal delivery and administration by the rectal, colonic, vaginal, intranasal or respiratory tract route.

The terms “chimeric protein”, “fusion protein”, “fusion polypeptide”, and “chimeric polypeptide” are used interchangeably herein and refer to a fusion of a first amino acid sequence encoding a polypeptide with a second amino acid sequence defining a domain (e.g., polypeptide portion) foreign to or heterologous with and not substantially homologous with the domain of the first polypeptide. A chimeric protein may present a foreign domain, which is found (albeit in a different protein) in an organism, which also expresses the first protein, or it may be an “interspecies”, “intergenic”, etc. fusion of protein structures expressed by different kinds of organisms.

An “effective amount” of an agent or therapeutic peptide is an amount sufficient to achieve a desired therapeutic or pharmacological effect, such as an amount that is capable of activating the growth of neurons. An effective amount of an agent as defined herein may vary according to factors, such as the disease state, age, and weight of the subject, and the ability of the agent to elicit a desired response in the subject. Dosage regimens may be adjusted to provide the optimum therapeutic response. An effective amount is also one in which any toxic or detrimental effects of the active compound are outweighed by the therapeutically beneficial effects.

The term “expression” refers to the process by which nucleic acid is translated into peptides or is transcribed into RNA, which, for example, can be translated into peptides, polypeptides or proteins. If the nucleic acid is derived from genomic DNA, expression may, if an appropriate eukaryotic host cell or organism is selected, include splicing of the mRNA. For heterologous nucleic acid to be expressed in a host cell, it must initially be delivered into the cell and then, once in the cell, ultimately reside in the nucleus.

The term “genetic therapy” and grammatical variants thereof (e.g., “gene therapy”), involves the transfer of heterologous DNA to cells of a mammal, particularly a human, with a disorder or conditions for which therapy or diagnosis is sought. The DNA is introduced into the selected target cells in a manner such that the heterologous DNA is expressed and a therapeutic product encoded thereby is produced. Alternatively, the heterologous DNA may in some manner mediate expression of DNA that encodes the therapeutic product; it may encode a product, such as a peptide or RNA that in some manner mediates, directly or indirectly, expression of a therapeutic product. Genetic therapy may also be used to deliver nucleic acid encoding a gene product to replace a defective gene or supplement a gene product produced by the mammal or the cell in which it is introduced. The heterologous DNA encoding the therapeutic product may be modified prior to introduction into the cells of the afflicted host in order to enhance or otherwise alter the product or expression thereof.

The term “gene” or “recombinant gene” refers to a nucleic acid comprising an open reading frame encoding a polypeptide, including both exon and (optionally) intron sequences.

The term “heterologous nucleic acid sequence” is typically DNA that encodes RNA and proteins that are not normally produced in vivo by the cell in which it is expressed or that mediates or encodes mediators that alter expression of endogenous DNA by affecting transcription, translation, or other regulatable biochemical processes. A heterologous nucleic acid sequence may also be referred to as foreign DNA. Any DNA that one of skill in the art would recognize or consider as heterologous or foreign to the cell in which it is expressed is herein encompassed by heterologous DNA. Examples of heterologous DNA include, but are not limited to, DNA that encodes traceable marker proteins, such as a protein that confers drug resistance, DNA that encodes therapeutically effective substances, and DNA that encodes other types of proteins, such as antibodies. Antibodies that are encoded by heterologous DNA may be secreted or expressed on the surface of the cell in which the heterologous DNA has been introduced.

The term “heterologous polypeptide” as used herein refers to a non-naturally or foreign occurring polypeptide compared to a protein tyrosine phosphatase receptor δ (Ptprd) polypeptide of a fusion or chimeric protein or polypeptide. Accordingly, a heterologous polypeptide of a fusion or chimeric protein or polypeptide is not found in nature in at least one Ptprd polypeptide of the fusion or chimeric protein or polypeptide, so the heterologous polypeptide is non-naturally occurring with respect the at least one Ptprd polypeptide. For example, a fusion or chimeric protein or polypeptide includes a Ptprd polypeptide that is a portion of a Ptprd polypeptide and the fusion or chimeric protein or polypeptide includes a heterologous polypeptide, and the heterologous polypeptide is non-naturally occurring compared to the Ptprd polypeptide in the absence of the heterologous polypeptide (e.g., the Ptprd polypeptide without or prior to including (e.g., insertion or fusion of) the heterologous polypeptide and the Ptprd polypeptide). In some embodiments, the heterologous polypeptide is heterologous to the Ptprd protein from which the Ptprd polypeptide is a portion of.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into a target tissue, such that it enters the animal's system and, thus, is subject to metabolism and other like processes, for example, subcutaneous administration.

The term “patient” or “subject” or “animal” or “host” or “individual” refers to any mammal. The subject may be a human, but can also be a mammal in need of veterinary treatment, e.g., domestic animals (e.g., dogs, cats, and the like), farm animals (e.g., cows, sheep, fowl, pigs, horses, and the like) and laboratory animals (e.g., rats, mice, guinea pigs, and the like).

The terms “polynucleotide sequence”, “nucleotide sequence”, and “nucleic acid sequence” are also used interchangeably herein.

The terms “peptide” or “polypeptide” are used interchangeably herein and refer to compounds consisting of from about 2 to about 90 amino acid residues, inclusive, wherein the amino group of one amino acid is linked to the carboxyl group of another amino acid by a peptide bond. A peptide can be, for example, derived or removed from a native protein by enzymatic or chemical cleavage, or can be prepared using conventional peptide synthesis techniques (e.g., solid phase synthesis) or molecular biology techniques (see Sambrook et al., MOLECULAR CLONING: LAB. MANUAL (Cold Spring Harbor Press, Cold Spring Harbor, NY, 1989)). A “peptide” can comprise any suitable L- and/or D-amino acid, for example, common a-amino acids (e.g., alanine, glycine, valine), non-a-amino acids (e.g., P-alanine, 4-aminobutyric acid, 6aminocaproic acid, sarcosine, statine), and unusual amino acids (e.g., citrulline, homocitruline, homoserine, norleucine, norvaline, ornithine). The amino, carboxyl and/or other functional groups on a peptide can be free (e.g., unmodified) or protected with a suitable protecting group. Suitable protecting groups for amino and carboxyl groups, and means for adding or removing protecting groups are known in the art. See, e.g., Green &amp; Wuts, PROTECTING GROUPS IN ORGANIC SYNTHESIS (John Wiley &amp; Sons, 1991). The functional groups of a peptide can also be derivatized (e.g., alkylated) using art-known methods.

Peptides can be synthesized and assembled into libraries comprising a few too many discrete molecular species. Such libraries can be prepared using well-known methods of combinatorial chemistry, and can be screened as described herein or using other suitable methods to determine if the library comprises peptides which can sequester asprosin. Such peptides can then be isolated by suitable means.

The term “peptidomimetic”, refers to a protein-like molecule designed to mimic a peptide. Peptidomimetics typically arise either from modification of an existing peptide, or by designing similar systems that mimic peptides, such as peptoids and β-peptides. Irrespective of the approach, the altered chemical structure is designed to advantageously adjust the molecular properties such as, stability or biological activity. These modifications involve changes to the peptide that do not occur naturally (such as altered backbones and the incorporation of nonnatural amino acids).

The terms “portion”, “fragment”, “variant”, “derivative” and “analog”, when referring to a polypeptide include any polypeptide that retains at least some biological activity referred to herein (e.g., inhibition of an interaction such as binding). Polypeptides as described herein may include portion, fragment, variant, or derivative molecules without limitation, as long as the polypeptide still serves its function. Polypeptides or portions thereof of the present invention may include proteolytic fragments, deletion fragments and in particular, or fragments that more easily reach the site of action when delivered to an animal.

In some embodiments, a “portion” or “fragment” polypeptide (including a domain) will be understood to mean a polypeptide of reduced length (e.g., reduced by 1, 2, 3, 4, 5, 6, 7, 8, 9, 10, 11, 12, 13, 14, 15, 16, 17, 18, 19, 20 or more residue(s) (e.g., peptide(s)) relative to a reference polypeptide, respectively, and comprising, consisting essentially of and/or consisting of a polypeptide of contiguous residues, respectively, identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to the reference polypeptide.

Different nucleic acids or proteins having homology are referred to herein as “homologues.” The term homologue includes homologous sequences from the same and other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the compositions and methods described herein further comprise homologues to the nucleotide sequences and polypeptides of this invention. “Orthologous” and “orthologs” as used herein, refers to homologous nucleotide sequences and/or amino acid sequences in different species that arose from a common ancestral gene during speciation. A homologue or ortholog of a nucleotide sequence of this invention has a substantial sequence identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to the nucleotide sequence described herein.

The term “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

The term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence as compared to a reference polypeptide.

The phrase “substantially identical,” or “substantial identity” in the context of two nucleic acid molecules, nucleotide sequences, polypeptide sequences, or protein sequences, refers to two or more sequences or subsequences that have at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% nucleotide or amino acid residue identity, when compared and aligned for maximum correspondence, as measured using one of the following sequence comparison algorithms or by visual inspection. In some embodiments, the substantial identity exists over a region of consecutive nucleotides of a nucleotide sequence of the invention that is about 10 nucleotides to about 20 nucleotides, about 10 nucleotides to about 25 nucleotides, about 10 nucleotides to about 30 nucleotides, about 15 nucleotides to about 25 nucleotides, about 30 nucleotides to about 40 nucleotides, about 50 nucleotides to about 60 nucleotides, about 70 nucleotides to about 80 nucleotides, about 90 nucleotides to about 100 nucleotides, or more nucleotides in length, and any range therein, up to the full length of the sequence. In some embodiments, the nucleotide sequences can be substantially identical over at least about 20 nucleotides (e.g., about 20, 21, 22, 23, 24, 25, 26, 27, 28, 29, 30, 31, 32, 33, 34, 35, 36, 37, 38, 39, 40 nucleotides). In some embodiments, a substantially identical nucleotide or protein sequence performs substantially the same function as the nucleotide (or encoded protein sequence) to which it is substantially identical.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG. Wisconsin Package (Accelrys Inc., San Diego, Calif.). An “identity fraction” for aligned segments of a test sequence and a reference sequence is the number of identical components which are shared by the two aligned sequences divided by the total number of components in the reference sequence segment, e.g., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Two nucleotide sequences may also be considered substantially complementary when the two sequences hybridize to each other under stringent conditions. In some representative embodiments, two nucleotide sequences considered to be substantially complementary hybridize to each other under highly stringent conditions.

“Stringent hybridization conditions” and “stringent hybridization wash conditions” in the context of nucleic acid hybridization experiments such as Southern and Northern hybridizations are sequence dependent, and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tₘ) for the specific sequence at a defined ionic strength and pH.

A polynucleotide and/or recombinant nucleic acid construct described herein can be codon optimized for expression. In some embodiments, a polynucleotide, nucleic acid construct, expression cassette, and/or vector described herein (e.g., that comprises/encodes a fusion or chimeric protein or polypeptide) may be codon optimized for expression in an organism (e.g., an animal, a plant, a fungus, an archaeon, or a bacterium). In some embodiments, the codon optimized nucleic acid constructs, polynucleotides, expression cassettes, and/or vectors of the invention have about 70% to about 99.9% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%. 99.9% or 100%) identity or more to the reference nucleic acid constructs, polynucleotides, expression cassettes, and/or vectors but which have not been codon optimized.

In any of the embodiments described herein, a polynucleotide or nucleic acid construct described herein may be operatively associated with a variety of promoters and/or other regulatory elements for expression in an organism or cell thereof. Thus, in some embodiments, a polynucleotide or nucleic acid construct described herein may further comprise one or more promoters, introns, enhancers, and/or terminators operably linked to one or more nucleotide sequences. In some embodiments, a promoter may be operably associated with an intron. In some embodiments, a promoter associated with an intron maybe referred to as a “promoter region”.

A polynucleotide sequence (DNA, RNA) is “operatively linked” to an expression control sequence when the expression control sequence controls and regulates the transcription and translation of that polynucleotide sequence. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

The term “linked,” or “fused” in reference to polypeptides, refers to the attachment of one polypeptide to another. A polypeptide may be linked or fused to another polypeptide (at the N-terminus or the C-terminus) directly (e.g., via a peptide bond) or through a linker (e.g., a peptide linker).

The term “linker” in reference to polypeptides is art-recognized and refers to a chemical group, or a molecule linking two molecules or moieties, e.g., two domains of a fusion polypeptide protein. A linker may be comprised of a single linking molecule (e.g., a single amino acid) or may comprise more than one linking molecule. In some embodiments, the linker can be an organic molecule, group, polymer, or chemical moiety such as a bivalent organic moiety. In some embodiments, the linker may be an amino acid or it may be a peptide. In some embodiments, the linker is a peptide.

A “promoter” is a nucleotide sequence that controls or regulates the transcription of a nucleotide sequence (e.g., a coding sequence) that is operably associated with the promoter. The coding sequence controlled or regulated by a promoter may encode a polypeptide and/or a functional RNA. Typically, a “promoter” refers to a nucleotide sequence that contains a binding site for RNA polymerase II and directs the initiation of transcription. In general, promoters are found 5′, or upstream, relative to the start of the coding region of the corresponding coding sequence.

Promoters can include, for example, constitutive, inducible, temporally regulated, developmentally regulated, chemically regulated, tissue-preferred and/or tissue-specific promoters for use in the preparation of recombinant nucleic acid molecules, e.g., “synthetic nucleic acid constructs” or “protein-RNA complex.” These various types of promoters are known in the art.

The choice of promoter may vary depending on the temporal and spatial requirements for expression, and also may vary based on the host cell to be transformed. Promoters for many different organisms are well known in the art. Based on the extensive knowledge present in the art, the appropriate promoter can be selected for the particular host organism of interest. Thus, for example, much is known about promoters upstream of highly constitutively expressed genes in model organisms and such knowledge can be readily accessed and implemented in other systems as appropriate.

The term “recombinant,” as used herein, means that a protein is derived from a prokaryotic or eukaryotic expression system.

The term “therapeutically effective” means that the amount of the composition used is of sufficient quantity to ameliorate one or more causes, symptoms, or sequelae of a disease or disorder. Such amelioration only requires a reduction or alteration, not necessarily elimination, of the causes, symptoms, or sequelae of a disease or disorder.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “vector” refers to a nucleic acid molecule capable of transporting another nucleic acid to which it has been linked. Preferred vectors are those capable of one or more of, autonomous replication and expression of nucleic acids to which they are linked. Vectors capable of directing the expression of genes to which they are operatively linked are referred to herein as “expression vectors”.

The term “wild type” (or “WT”) refers to the naturally-occurring polynucleotide sequence encoding a protein, or a portion thereof, or protein sequence, or portion thereof, respectively, as it normally exists in vivo. As used herein, the term “nucleic acid” refers to polynucleotides, such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

The agents, compounds, compositions, polypeptides, proteins, etc. used in the methods described herein are considered to be purified and/or isolated prior to their use. Purified materials are typically “substantially pure”, meaning that a nucleic acid, polypeptide or fragment thereof, or other molecule has been separated from the components that naturally accompany it. Typically, the polypeptide is substantially pure when it is at least 60%, 70%, 80%, 90%, 95%, or even 99%, by weight, free from the proteins and other organic molecules with which it is associated naturally. For example, a substantially pure polypeptide may be obtained by extraction from a natural source, by expression of a recombinant nucleic acid in a cell that does not normally express that protein, or by chemical synthesis. “Isolated materials” have been removed from their natural location and environment. In the case of an isolated or purified domain or protein fragment, the domain or fragment is substantially free from amino acid sequences that flank the protein in the naturally-occurring sequence. The term “isolated DNA” means DNA has been substantially freed of the genes that flank the given DNA in the naturally occurring genome. Thus, the term “isolated DNA” encompasses, for example, cDNA, cloned genomic DNA, and synthetic DNA.

The term “insulin resistance” refers to the condition in which normal amounts of insulin are inadequate to produce a normal insulin response from fat, muscle and liver cells. Insulin resistance in fat cells results in hydrolysis of stored triglycerides, which elevates free fatty acids in the blood plasma. Insulin resistance in muscle reduces glucose uptake whereas insulin resistance in liver reduces glucose storage, with both effects serving to elevate blood glucose. High plasma levels of insulin and glucose due to insulin resistance often leads to metabolic syndrome and type 2 diabetes.

The term “metabolic disorders” refers to a group of identified disorders in which errors of metabolism, imbalances in metabolism, or sub-optimal metabolism occur. The metabolic disorders as described herein also include diseases that can be treated through the modulation of metabolism, although the disease itself may or may not be caused by a specific metabolic defect. Such metabolic disorders may involve, for example, glucose oxidation pathways.

The term “metabolic syndrome” refers a combination of medical disorders that increase one's risk for cardiovascular disease and diabetes. It is known under various other names, such as (metabolic) syndrome X, insulin resistance syndrome, Reaven's syndrome. Symptoms and features are fasting hyperglycemia, diabetes mellitus type 2 or impaired fasting glucose, impaired glucose tolerance, or insulin resistance; high blood pressure; central obesity (also known as visceral, male-pattern or apple-shaped adiposity), overweight with fat deposits mainly around the waist; decreased HDL cholesterol; elevated triglycerides; and elevated uric acid levels. Associated diseases and signs are: fatty liver (especially in concurrent obesity), progressing to non-alcoholic fatty liver disease, polycystic ovarian syndrome, hemochromatosis (iron overload); and acanthosis nigricans (a skin condition featuring dark patches).

The term “metabolic disease” refers to a group of identified disorders in which errors of metabolism, imbalances in metabolism, or sub-optimal metabolism occur. The metabolic diseases as described herein also include diseases that can be treated through the modulation of metabolism, although the disease itself may or may not be caused by a specific metabolic defect. Such metabolic diseases may involve, for example, glucose and fatty acid oxidation pathways.

The term “obesity” as used herein is defined in the WHO classifications of weight. Underweight is less than 18.5 BMI (thin); healthy is 18.5-24.9 BMI (normal); grade 1 overweight is 25.0-29.9 BMI (overweight); grade 2 overweight is 30.0-39.0 BMI (obesity); grade 3 overweight is greater than or equal to 40.0 BMI. BMI is body mass index (morbid obesity) and is kg/m². Waist circumference can also be used to indicate a risk of metabolic complications. Waist circumference can be measured (in cm) at midpoint between the lower border of ribs and the upper border of the pelvis. Other measures of obesity include, but are not limited to, skinfold thickness and bioimpedance, which is based on the principle that lean mass conducts current better than fat mass because it is primarily an electrolyte solution.

The term “obesity related condition” refers to any disease or condition that is caused by or associated with (e.g., by biochemical or molecular association) obesity or that is caused by or associated with weight gain and/or related biological processes that precede clinical obesity. Examples of obesity-related conditions include, but are not limited to, diabetes (e.g., type 1 diabetes, type 2 diabetes, and gestational diabetes), Syndrome X, hyperglycemia, hyperinsulinemia, impaired glucose tolerance, impaired fasting glucose, dyslipidemia, hypertriglyceridemia, insulin resistance, hypercholesterolemia, atherosclerosis, coronary artery disease, peripheral vascular disease, and hypertension.

“Diabetic complications” is the dysfunction of other tissue/organs of the body induced by chronic hyperglycemia, such as diabetic nephropathy, diabetic neuropathy, diabetic feet (foot ulcers and low blood circulation) and eye lesions (retinopathy). Diabetes also increases the risks of heart disease and osteoarticular diseases. The other long-term complications of diabetes comprise skin, digestive, sexual function, teeth and gums disease. “Dyslipidemia” is a lipoprotein metabolic disorder, comprising the over synthesis or defect of lipoprotein. Dyslipidemia can exhibit as the elevated concentration of total cholesterol, low density lipoprotein (LDL) cholesterol and triglycerides, and the reduced concentration of high density lipoprotein (HDL) cholesterol.

“Nonalcoholic fatty liver disease (NAFLD)” is a liver disease which is not associated with abused alcohol consumption and is characterized in hepatocellular steatosis.

“Nonalcoholic steatohepatitis (NASH)” is a liver disease which is not associated with abused alcohol consumption and is characterized in hepatocellular steatosis accompanied by lobular inflammation and fibrosis.

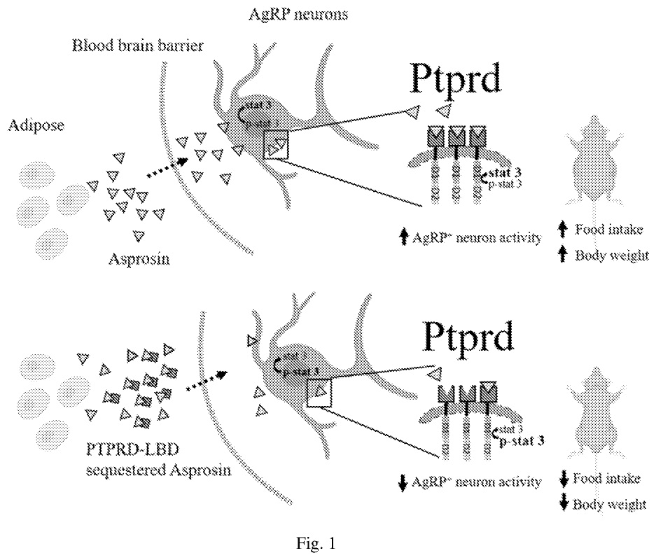

Embodiments described herein relate to compositions and methods of inhibiting asprosin mediated orexigenesis and/or glucogenesis, including orexigenic and/or glucogenic activity or function of asprosin in a subject in need thereof, and particularly relates to compositions and methods of treating or preventing one or more of insulin resistance, obesity, an obesity related condition, diabetes, a metabolic disease or disorder, such as metabolic syndrome, and being overweight in a subject in need thereof. We identified Protein Tyrosine Phosphatase Receptor δ (Ptprd) (SEQ ID NO: 1), a membrane bound phosphatase receptor as the orexigenic and/or glucogenic asprosin-receptor. In mice, genetic ablation of Ptprd recapitulates the hypophagic and markedly lean phenotype associated with genetic asprosin deficiency and renders null mice unresponsive to the orexigenic effects of asprosin. AgRP⁺ neuron-specific Ptprd ablation renders neurons unresponsive to asprosin-mediated activation and leads to protection of mice from diet-induced obesity. As illustrated schematically in FIG. 1, we also found that a peptide comprising a portion of the asprosin ligand binding domain of Ptprd (i.e., Ptprd-ligand binding domain or Ptprd-lbd), when introduced ectopically into or administered in the circulation of obese mice, binds to and sequesters plasma asprosin leading to improvements in appetite, body weight and blood glucose.

Accordingly, in some embodiments, a method of inhibiting asprosin mediated orexogenesis and/or glucogenesis in a subject in need thereof includes administering to the subject a therapeutically effective amount of an agent that inhibits asprosin mediated Ptprd signaling or activity. In some embodiments, the agent can include a therapeutic peptide that is a peptide mimetic of the asprosin ligand binding domain of Ptprd (i.e., Ptprd-lbd). The therapeutic peptide or peptide mimetic of the asprosin ligand binding domain of Ptprd can have an amino acid sequence substantially identical to an extracellular portion of the amino acid sequence of Ptprd that binds to asprosin. Peptide mimetics of the Ptprd-lbd when ectopically introduced into the circulation of a subject in need thereof inhibit asprosin induced or mediated Ptprd signaling in neural cells resulting in a corresponding decrease in appetite, body weight, blood glucose levels. In some embodiments, the peptide mimetic or therapeutic peptide can bind to and sequester asprosin in the circulation of the subject.

In some embodiments, the therapeutic peptide can have an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to at least about 10, at least about 20, at least about 30, at least about 40, at least about 50, at least about 60, at least about 70, at least about 80, at least about 90, at least about 100, at least about 150, at least about 200, at least about 300, at least about 400, at least about 500, at least about 600, at least about 700, at least about 800, at least about 900, or at least 1000 consecutive amino acids of SEQ ID NO: 2.

For example, the therapeutic peptide can have an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to about 10 to about 1240, about 20 to about 1220,about 30 to about 1210, about 40 to about 1200, about 50 to about 1190, about 60 to about 1180, about 70 to about 1170, about 80 to about 1160, about 90 to about 1180, about 100 to about 1150, about 200 to about 1100, about 300 to about 1000, about 400 to about 900, or about 500 to about 800 consecutive amino acids of SEQ ID NO: 2.

In other embodiments, the therapeutic peptide can have an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 2.

In still other embodiments, the therapeutic peptide can have an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to at least about 10, at least about 20, at least about 30, at least about 40, at least about 50, at least about 60, at least about 70, at least about 80, at least about 90, at least about 100, at least about 150, at least about 200 consecutive amino acids of the extracellular Ig domain of Ptprd.

In one example, the therapeutic peptide can have an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to at least about 10, at least about 20, at least about 30, at least about 40, at least about 50, at least about 60, at least about 70, at least about 80, at least about 90, at least about 100, at least about 150, at least about 200 consecutive amino acids of SEQ ID NO: 3.

In another example, the therapeutic peptide can have an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to at least about 10, at least about 20, at least about 30, at least about 40, at least about 50, at least about 60, at least about 70, at least about 80 consecutive amino acids of SEQ ID NO: 4.

In another example, the therapeutic peptide can have an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to at least about 10, at least about 20, at least about 30, at least about 40, at least about 50, at least about 60, at least about 70, at least about 80, at least about 90 consecutive amino acids of SEQ ID NO: 19.

In another example, the therapeutic peptide can have an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to at least about 10, at least about 20, at least about 30, at least about 40, at least about 50, at least about 60, at least about 70, at least about 80, at least about 90 consecutive amino acids of SEQ ID NO: 20.

In some embodiments, the therapeutic peptide has a binding affinity K_{D} to asprosin less than about 10 μM, less than about 1 μM, less than about 500 nM, less than about 400 nM, less than about 300 nM, less than about 200 nM, less than about 100 nM, less than about 10 nM, less about 1 nM, or less than about 500 pM.

The therapeutic peptides described herein can be subject to other various changes, substitutions, insertions, and deletions where such changes provide for certain advantages in its use. In this regard, therapeutic peptides that have an amino acid sequence substantially identical to an extracellular portion of the amino acid sequence of Ptprd that binds to asprosin can correspond to or be substantially homologous with, rather than be identical to, the sequence of a recited polypeptide where one or more changes are made and it retains the ability to inhibit or reduce one or more of the activity, signaling, and/or function of asprosin orexigenesis and/or glucogenesis.

The therapeutic peptide can be in any of a variety of forms of polypeptide derivatives that include amides, conjugates with proteins, cyclized polypeptides, polymerized polypeptides, analogs, fragments, chemically modified polypeptides and the like derivatives.

The therapeutic peptide can also include conservative substitutions of amino acid residues. It will be appreciated that the conservative substitution can also include the use of a chemically derivatized residue in place of a non-derivatized residue provided that such peptide displays the requisite binding activity.

“Chemical derivative” refers to a subject peptide having one or more residues chemically derivatized by reaction of a functional side group. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those polypeptides, which contain one or more naturally occurring amino acid derivatives of the twenty standard amino acids. For example: 4-hydroxyproline may be substituted for proline; 5-hydroxylysine may be substituted for lysine; 3-methylhistidine may be substituted for histidine; homoserine may be substituted for serine; and ornithine may be substituted for lysine. Polypeptides described herein may also include any polypeptide having one or more additions and/or deletions or residues relative to the sequence of a polypeptide whose sequence is shown herein, so long as the requisite activity is maintained.

One or more of peptides of the therapeutic peptides described herein can also be modified by natural processes, such as posttranslational processing, and/or by chemical modification techniques, which are known in the art. Modifications may occur in the peptide including the peptide backbone, the amino acid side-chains and the amino or carboxy termini. It will be appreciated that the same type of modification may be present in the same or varying degrees at several sites in a given peptide. Modifications comprise for example, without limitation, acetylation, acylation, addition of acetomidomethyl (Acm) group, ADP-ribosylation, amidation, covalent attachment to fiavin, covalent attachment to a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cystine, formation of pyroglutamate, formylation, gamma-carboxylation, glycosylation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation and ubiquitination (for reference see, Protein-structure and molecular properties, 2nd Ed., T. E. Creighton, W. H. Freeman and Company, New-York, 1993).

Peptides and/or proteins described herein may also include, for example, biologically active mutants, variants, fragments, chimeras, and analogues; fragments encompass amino acid sequences having truncations of one or more amino acids, wherein the truncation may originate from the amino terminus (N-terminus), carboxy terminus (C-terminus), or from the interior of the protein. Analogues of the invention involve an insertion or a substitution of one or more amino acids. Variants, mutants, fragments, chimeras and analogues may function as inhibitors of asprosin mediated orexigenesis and/or glucogenesis (without being restricted to the present examples).

The therapeutic polypeptides described herein may be prepared by methods known to those skilled in the art. The peptides and/or proteins may be prepared using recombinant DNA. For example, one preparation can include cultivating a host cell (bacterial or eukaryotic) under conditions, which provide for the expression of peptides and/or proteins within the cell.

The purification of the polypeptides may be done by affinity methods, ion exchange chromatography, size exclusion chromatography, hydrophobicity or other purification technique typically used for protein purification. The purification step can be performed under non-denaturating conditions. On the other hand, if a denaturating step is required, the protein may be renatured using techniques known in the art.

In some embodiments, the therapeutic peptide is an exogenous peptide that can be recombinantly produced and systemically administered to the subject by, for example, parenteral or intravenous administration.

In some embodiments, the therapeutic peptide includes at least one heterologous or foreign moiety, such as a heterologous or foreign polypeptide. The at least one heterologous polypeptide can include, for example, an antibody or antigen binding fragment thereof, a glucagon-like peptide-1 receptor (GLP-1R) agonist, a Fc portion of an immunoglobulin, an albumin peptide, an albumin binding domain (ABD), a signal peptide, or a combination thereof.

In some embodiments, the heterologous moiety is fused to the N-terminus or C-terminus of the therapeutic peptide. In other embodiments, the heterologous moiety is inserted between two amino acids within the therapeutic peptide.

In other embodiments, the therapeutic peptide can further comprise two, three, four, five, six, seven, or eight heterologous sequences. In some embodiments, all the heterologous moieties are identical. In some embodiments, at least one heterologous moiety is different from the other heterologous moieties. In some embodiments, the disclosure can comprise two, three, four, five, six, or more than seven heterologous moieties in tandem.

In some embodiments, the heterologous moiety increases the half-life (is a “half-life extender”) of the therapeutic peptide.

In some embodiments, the heterologous moiety is a peptide or a polypeptide with either unstructured or structured characteristics that are associated with the prolongation of in vivo half-life when incorporated in the therapeutic peptide. Non-limiting examples include albumin, albumin fragments, Fc fragments of immunoglobulins, the C-terminal peptide (CTP) of the β subunit of human chorionic gonadotropin, a HAP sequence, an XTEN sequence, a transferrin or a fragment thereof, a PAS polypeptide, polyglycine linkers, polyserine linkers, albumin-binding moieties, or any fragments, derivatives, variants, or combinations of these polypeptides.

In one particular embodiment, the heterologous polypeptide includes an immunoglobulin constant region or a portion thereof, transferrin, albumin, or a PAS sequence. In some aspects, a heterologous moiety includes von Willebrand factor or a fragment thereof. In other related aspects a heterologous polypeptide can include an attachment site (e.g., a cysteine amino acid) for a non-polypeptide moiety such as polyethylene glycol (PEG), hydroxyethyl starch (HES), polysialic acid, or any derivatives, variants, or combinations of these elements. In some aspects, a heterologous moiety comprises a cysteine amino acid that functions as an attachment site for a non-polypeptide moiety such as polyethylene glycol (PEG), hydroxyethyl starch (HES), polysialic acid, or any derivatives, variants, or combinations of these elements.

In one specific embodiment, a first heterologous polypeptide is a half-life extending molecule which is known in the art, and a second heterologous moiety is a half-life extending molecule which is known in the art. In certain embodiments, the first heterologous polypeptide (e.g., a first Fc polypeptide) and the second heterologous polypeptide (e.g., a second Fc polypeptide) are associated with each other to form a dimer. In one embodiment, the second heterologous polypeptide is a second Fc polypeptide, wherein the second Fc polypeptide is linked to or associated with the first heterologous polypeptide, e.g., the first Fc polypeptide. For example, the second heterologous polypeptide (e.g., the second Fc polypeptide) can be linked to the first heterologous moiety (e.g., the first Fc polypeptide) by a linker or associated with the first heterologous moiety by a covalent or non-covalent bond.

In some embodiments, the heterologous polypeptide comprises, consists essentially of, or consists of at least about 10, at least about 100, at least about 200, at least about 300, at least about 400, at least about 500, at least about 600, at least about 700, at least about 800, at least about 900, at least about 1000, at least about 1100, at least about 1200, at least about 1300, at least about 1400, at least about 1500, at least about 1600, at least about 1700, at least about 1800, at least about 1900, at least about 2000, at least about 2500, at least about 3000, or at least about 4000 amino acids. In other embodiments, the heterologous polypeptide comprises, consists essentially of, or consists of about 100 to about 200 amino acids, about 200 to about 300 amino acids, about 300 to about 400 amino acids, about 400 to about 500 amino acids, about 500 to about 600 amino acids, about 600 to about 700 amino acids, about 700 to about 800 amino acids, about 800 to about 900 amino acids, or about 900 to about 1000 amino acids.

In certain embodiments, a heterologous polypeptide improves one or more pharmacokinetic properties of the therapeutic peptide without significantly affecting its biological activity or function.

In certain embodiments, a heterologous polypeptide increases the in vivo and/or in vitro half-life of the therapeutic peptide. In other embodiments, a heterologous polypeptide facilitates visualization or localization of the therapeutic peptide. Visualization and/or location of the therapeutic peptide can be in vivo, in vitro, ex vivo, or combinations thereof.

In other embodiments, a heterologous polypeptide increases stability of the therapeutic peptide. As used herein, the term “stability” refers to an art-recognized measure of the maintenance of one or more physical properties of the therapeutic protein in response to an environmental condition (e.g., an elevated or lowered temperature). In certain aspects, the physical property can be the maintenance of the covalent structure of the therapeutic peptide (e.g., the absence of proteolytic cleavage, unwanted oxidation or deamidation). In other aspects, the physical property can also be the presence of the therapeutic peptide in a properly folded state (e.g., the absence of soluble or insoluble aggregates or precipitates). In one aspect, the stability of the therapeutic peptide is measured by assaying a biophysical property of the therapeutic protein, for example thermal stability, pH unfolding profile, stable removal of glycosylation, solubility, biochemical function (e.g., ability to bind to a protein, receptor or ligand), etc., and/or combinations thereof. In another aspect, biochemical function is demonstrated by the binding affinity of the interaction. In one aspect, a measure of protein stability is thermal stability, i.e., resistance to thermal challenge. Stability can be measured using methods known in the art, such as, HPLC (high performance liquid chromatography), SEC (size exclusion chromatography), DLS (dynamic light scattering), etc. Methods to measure thermal stability include, but are not limited to differential scanning calorimetry (DSC), differential scanning fluorimetry (DSF), circular dichroism (CD), and thermal challenge assay.

In some embodiments, the heterologous moiety or polypeptide can include an Fc portion of an immunoglobulin. The Fc portion of an immunoglobulin can be linked to the therapeutic peptide to form a fusion or chimeric polypeptide or protein. Fusion or chimeric polypeptides or proteins that can combine the Fc regions of IgG with one or more domains of another protein, such as various cytokines and soluble receptors, are known. These chimeric proteins can be fusions of human Fc regions and human domains of another protein. These chimeric proteins would then be a “humanized Fc chimera”, which would be advantageous as a human therapeutic. (See, for example, Capon et al., Nature, 337:525-531, 1989; Chamow et al., Trends Biotechnol., 14:52-60, (1996); U.S. Pat. Nos. 5,116,964 and 5,541,087). The fusion polypeptide can be a homodimeric protein linked through cysteine residues in a hinge region of IgG Fc, resulting in a molecule similar to an IgG molecule without the CHI domains and light chains. Due to the structural homology, such Fc fusion proteins exhibit in vivo pharmacokinetic profile comparable to that of human IgG with a similar isotype. This approach has been applied to several therapeutically important cytokines, such as IL-2 and IFN-a, and soluble receptors, such as TNF-Rc and IL-5-Rc (See, for example, U.S. Pat. Nos. 5,349,053, 6,224,867 and 7,250,493).

In some embodiments, the therapeutic peptide-Fc fusion polypeptide or chimera is a chimeric molecule that includes a human sequence encoded extracellular portion of Ptprd fused to a human Fc fragment.

In other embodiments, the heterologous polypeptide can include a glucagon-like peptide 1 receptor (GLP-1R) agonist that is linked to the therapeutic peptide to form a fusion polypeptide or protein. GLP-1R agonist can include a peptide, which binds to and activates the GLP-1 receptor like GLP-1 (glucagon-like peptide 1). Physiological actions of GLP-1 and/or of the GLP-IR agonist are described e.g., in Nauck, M. A. et al. (1997) Exp. Clin. Endocrinol. Diabetes, 105, 187-195. These physiological actions in normal subjects, in particular humans, include e.g., glucose-dependent stimulation of insulin secretion, suppression of glucagon secretion, stimulation of (pro) insulin biosynthesis, reduction of food intake, deceleration of gastric emptying and/or equivocal insulin sensitivity.

Assays that can be used to discover GLP-IR agonists are described in, e.g., Thorkildsen, Chr. et al. (2003), Journal of Pharmacology and Experimental Therapeutics, 307, 490-496; Knudsen, L. B. et al. (2007), PNAS, 104, 937-942, No. 3; Chen, D. et al. (2007), PNAS, 104, 943-948, No. 3; or US2006/0003417 A1 (see e.g., Example 8). In short, in a “receptor binding assay”, a purified membrane fraction of eukaryotic cells harboring e.g. the human recombinant GLP-1 receptor, e.g. CHO, BHK or HEK293 cells, is incubated with the test compound or compounds in the presence of e.g., human GLP-1, e.g., GLP-1 (7-36) amide which is marked with e.g., ¹²⁵I (e.g. 80 kBq/pmol). Usually different concentrations of the test compound or compounds are used and the IC₅₀ values are determined as the concentrations diminishing the specific binding of human GLP-1.

In some embodiments, GLP-IR agonists are selected from a bioactive GLP-1, a GLP-1 analog or a GLP-1 substitute, as e.g. described in Drucker, D. J. (2006) Cell Metabolism, 3, 153-165; Thorkildsen, Chr. (2003; supra); Chen, D. et al. (2007; supra); Knudsen, L. B. et al. (2007; supra); Liu, J. et al. (2007) Neurochem Int., 51, 361-369, No. 6-7; Christensen, M. et al. (2009), Drugs, 12, 503-513; Maida, A. et al. (2008) Endocrinology, 149, 5670-5678, No. 11 and US2006/0003417. Examples of GLP-IR agonists include GLP-1 (7-37), GLP-1 (7-36) amide, exendin-4, liraglutide, CJC-1131, albugon, albiglutide, exenatide, exenatide-LAR, oxyntomodulin, lixisenatide, geniproside, a short peptide with GLP-IR agonistic activity and/or a small organic compound with GLP-IR agonistic activity.

Human GLP-1 (7-37) possesses the amino acid sequence of SEQ ID NO: 5. Human GLP-1 (7-36) amide possesses the amino acid sequence of SEQ ID NO: 6.

Other peptides with GLP-IR agonistic activity are disclosed in US 2006/0003417, US 2019/0085043, and small organic compounds with GLP-IR agonistic activity are disclosed in Chen et al. 2007, PNAS, 104, 943-948, No. 3 or Knudsen et al., 2007, PNAS, 104, 937-942.

The therapeutic peptide can linked directly to heterologous peptide or indirectly to the heterologous polypeptide with a linker. The linker can include a structural unit that is inserted in between two or more other units (e.g., two or more peptides or polypeptides or proteins or a peptide and a protein a polypeptide and a protein, a peptide and a polypeptide) and couple these two or more other units with each other to create one molecule. The coupling of the two units is preferably by covalent bond(s). The linker as used herein also refers to a structural unit that can be attached to the N- or C-terminus of two or more other units (e.g., two or more peptides or polypeptides or proteins or a peptide and a protein a polypeptide and a protein, a peptide and a polypeptide), wherein said two or more other units are directly coupled together. The linker as used herein also refers to combinations of the preceding definitions, i.e., one structural unit is inserted in between the two or more other units (e.g., two or more peptides or polypeptides or proteins or a peptide and a protein a polypeptide and a protein, a peptide and a polypeptide) and one or more further structural units is/are attached to the N- or C-terminus of two or more other units (e.g., two or more peptides or polypeptides or proteins or a peptide and a protein a polypeptide and a protein, a peptide and a polypeptide). The attachment of the structure unit to the N- or C-terminus of two or more other units is preferably by covalent bond(s).

In some embodiments, the linker can include, for example, additional residues that may be added at either terminus of a therapeutic peptide for the purpose of conveniently linking other the polypeptides, proteins or other molecules, such as detectable moieties, labels, solid matrices, or carriers.

Amino acid residue linkers are usually at least one residue and can be 2 or more residues, more often about 2 to about 1000 or more amino acids in length, for example, about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length (e.g., about 2 to about 40, about 2 to about 50, about 2 to about 60, about 4 to about 40, about 4 to about 50, about 4 to about 60, about 5 to about 40, about 5 to about 50, about 5 to about 60, about 9 to about 40, about 9 to about 50, about 9 to about 60, about 10 to about 40, about 10 to about 50, about 10 to about 60, or about 2, 3, 4, 5, 6, 7, 8, 9, 10, 11, 12, 13, 14, 15, 16, 17, 18, 19, 20, 21, 22, 23, 24, 25 amino acids to about 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 300, 400, 500, 600, 700, 800, 900 or about 1000 amino acids in length (e.g., about 105, 110, 115, 120, 130, 140 150 or more amino acids in length).

In some embodiments, the linker can be a flexible peptide linker that links the therapeutic peptide to other polypeptides, proteins, and/or molecules, such as detectable moieties, labels, solid matrices, or carriers. A flexible peptide linker can be about 20 or fewer amino acids in length. For example, a peptide linker can contain about 12 or fewer amino acid residues, e.g., 3, 4, 5, 6, 7, 8, 9, 10, 11, and 12. Typical amino acid residues used for linking are glycine, serine, tyrosine, cysteine, lysine, glutamic and aspartic acid, or the like. In some cases, a peptide linker comprises two or more of the following amino acids: glycine, serine, alanine, and threonine.

In some embodiments, a peptide linker may be a GS linker. In some embodiments, the peptide linker may comprise an amino acid sequence of (GGS)ₙ, GS, SG, GSSG (SEQ ID NO: 7), S (GGS) n (SEQ ID NO: 8), SGGS (SEQ ID NO: 9), or (GGGGS) n (SEQ ID NO: 10), wherein n is an integer of 1-20 (e.g., 1, 2, 3, 4, 5, 6, 7, 8, 9, 10, 11, 12, 13, 14, 15, 16, 17, 18, 19, or 20). In some embodiments, the peptide linker may comprise the amino acid sequence: SGGSGGSGGS (SEQ ID NO: 11). In some embodiments, the peptide linker may comprise the amino acid sequence: SGSETPGTSESATPES (SEQ ID NO: 12), also referred to as the XTEN linker. In some embodiments, the peptide linker may comprise the amino acid sequence: SGGSSGGSSGSETPGTSESATPESSGGSSGGS (SEQ ID NO: 13), also referred to as the GS-XTEN-GS linker.

In one embodiment of the fusion polypeptide or protein described herein the peptide linker can include a functional moiety conferring one or more additional functions beyond that of linking the therapeutic peptide and the heterologous moiety or polypeptide.

The linker can be added for improved or independent folding of one or both of the polypeptides forming the fusion polypeptide or protein and/or for avoiding sterical hindrance and/or for introducing further desired functionalities, e.g., entry sites for covalent attachment of additional moieties, tags for protein purification, protease cleavage sites, protein stabilization and/or half-life extension of the protein. Linkers can include between 0, 1 to 1000 amino acids. The linker can also be absent (i.e., 0 amino acids).

Typical linker types can e.g., be helical or non-helical, wherein helical linkers are thought to act as rigid spacers separating two domains and non-helical linkers contain proline or are rich in proline, which also leads to structural rigidity and isolation of the linker from the attached domains. This means that both linker types are likely to act as a scaffold to prevent unfavorable interactions between folding domains.

In some embodiments, the linker can a moiety conferring increased stability and/or half-life to the fusion polypeptide, such as a) an XTENylation, rPEG or PASylation or HESylation sequence or Elastin-like polypeptides (ELPs); b) an entry site for covalent modification of the fusion protein such as a cysteine or lysine residue; c) a moiety with intra- or extracellular targeting function such as a protein-binding scaffold (such as an antibody, antigen-binding fragment, or other proteinaceous non-antibody binding scaffold), a nucleic acid (such as an aptamer, PNA, DNA or the like); d) a protease cleavage site such as a Factor Xa cleavage site or a cleavage site for another (preferably extracellular) protease; or e) an albumin binding domain (ABD); or f) an amino acid sequence comprising one or more histidine (His linker, abbreviated as “His”) amino acids, for example HAHGHGHAH (SEQ ID NO: 14). The linker can also comprise one or more amino acids that do not confer additional functionality to the linker and a functionality-conferring moiety.

In some embodiments, the linker includes a factor Xa cleavage site that includes or consists of the sequence IEGR (SEQ ID NO:15) or a protease cleavage site that includes or consists of at least one arginine, such as GGGRR (SEQ ID NO: 16).

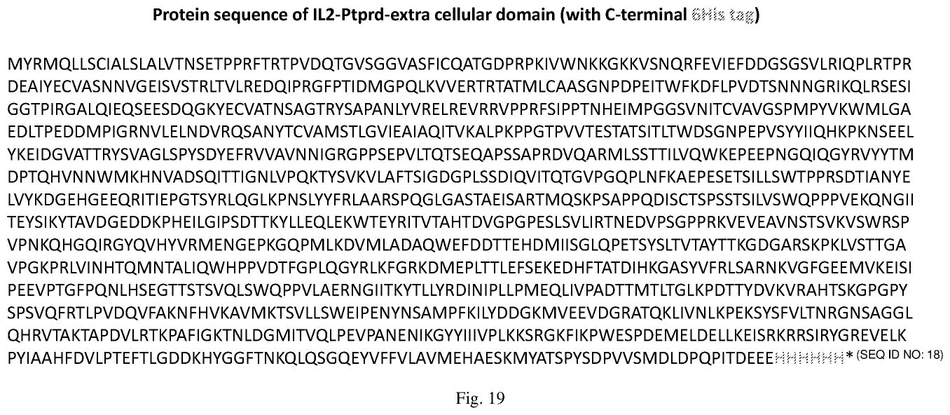

In some embodiments, the therapeutic peptide or fusion polypeptide comprising the therapeutic polypeptide may be modified to include a signal peptide that promotes secretion of the expressed therapeutic peptide or fusion polypeptide from a cell. The characteristics of the signal peptides are well known in the art, and the signal peptides conventionally having 16 to 30 amino acids, but they may include more or less number of amino acid residues. Conventional signal peptides consist of three regions of the basic N-terminal region, a central hydrophobic region, and a more polar C-terminal region. The signal peptide can include, for example, an IL2 or IL15 signal peptide. For example, the therapeutic peptide can have an IL2 signal peptide, such as described in SEQ ID NO: 17 and FIG. 19.

In some embodiments, the therapeutic peptide or fusion polypeptide comprising the therapeutic peptide and a heterologous polypeptide can be expressed from a cell, in vivo or ex vivo, using a vector that includes a nucleic acid encoding the therapeutic peptide or fusion polypeptide. A “vector” (sometimes referred to as gene delivery or gene transfer “vehicle”) refers to a macromolecule or complex of molecules comprising a polynucleotide to be delivered to the cell. The polynucleotide to be delivered may comprise a coding sequence of interest in gene therapy. Vectors include, for example, viral vectors (such as adenoviruses (Ad), adeno-associated viruses (AAV), and retroviruses), liposomes and other lipid-containing complexes, and other macromolecular complexes capable of mediating delivery of a polynucleotide to a target cell.

In some embodiments, the vector can include an expression cassette. The expression cassette can include a nucleic acid molecule which comprises the therapeutic peptide or fusion polypeptide coding sequences (e.g., coding sequences for the therapeutic peptide, heterologous polypeptide, and optional linker), promoter, and may include other regulatory sequences therefor, which cassette may be engineered into a genetic element and/or packaged into the capsid of a viral vector (e.g., a viral particle). Typically, such an expression cassette for generating a viral vector contains the therapeutic peptide and heterologous polypeptide sequences described herein flanked by packaging signals of the viral genome and other expression control sequences such as those described herein. Any of the expression control sequences can be optimized for a specific species using techniques known in the art including, e.g., codon optimization, as described herein.

The expression cassette typically contains a promoter sequence as part of the expression control sequences. For example, the promoter can include a liver-specific promoter thyroxin binding globulin (TBG). In another example, vectors described herein can include a CB7 promoter. CB7 is a chicken β-actin promoter with cytomegalovirus enhancer elements. Alternatively, other liver-specific promoters may be used. TTR minimal enhancer/promoter, alpha-antitrypsin promoter, LSP (845 nt) 25 (requires intron-less scAAV). Although less desired, other promoters, such as viral promoters, constitutive promoters, regulatable promoters (see, e.g., WO 2011/126808 and WO 2013/04943), or a promoter responsive to physiologic cues may be used may be utilized in the vectors described herein.

In addition to a promoter, an expression cassette and/or a vector may contain other appropriate control sequences, such as transcription initiation, termination, enhancer sequences, efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. Examples of suitable polyA sequences include, e.g., SV40, bovine growth hormone (bGH), and TK polyA. Examples of enhancers include, e.g., the alpha fetoprotein enhancer, the TTR minimal promoter/enhancer, LSP (TH-binding globulin promoter/alpha1-microglobulin/bikunin enhancer), amongst others. These control sequences can be operably linked to the therapeutic peptide sequences.

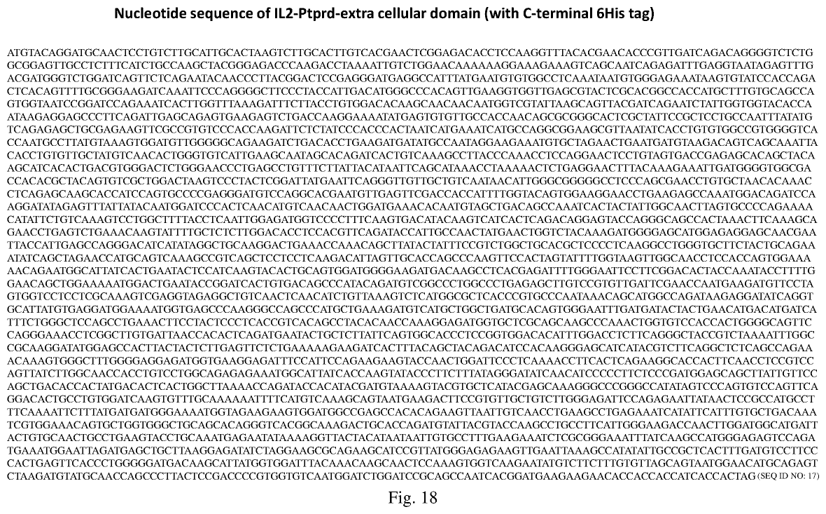

In some embodiments, the nucleic acid encoding the therapeutic peptides or fusion polypeptide may be modified to include a signal sequence that promotes secretion of the expressed therapeutic peptide from a cell. For example, the nucleic acid can include cDNA encoding the extracellular domain of Ptprd and IL2 signal sequence. The cDNA can have the nucleotide sequence of SEQ ID NO: 18, which is shown in FIG. 18. A number of such modifications are known in the art and can be applied by the skilled practitioner.

Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide (such as one or more transcriptional regulatory sequences). Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities.

Selectable markers can be positive, negative or bifunctional. Positive selectable markers allow selection for cells carrying the marker, whereas negative selectable markers allow cells carrying the marker to be selectively eliminated. A variety of such marker genes have been described, including bifunctional (i.e., positive/negative) markers (see, e.g., Lupton, S., WO 92/08796, published May 29, 1992; and Lupton, S., WO 94/28143, published Dec. 8, 1994). Such marker genes can provide an added measure of control that can be advantageous in gene therapy contexts. A large variety of such vectors are known in the art and are generally available.

In some embodiments, the vector can include an adeno-associated virus (AAV) viral vector. An AAV viral vector is an AAV DNase-resistant particle having an AAV protein capsid into which is packaged nucleic acid sequences for delivery to target cells. An AAV capsid is composed of 60 capsid (cap) protein subunits, VP1, VP2, and VP3, that are arranged in an icosahedral symmetry in a ratio of approximately 1:1:10 to 1:1:20, depending upon the selected AAV. AAV serotypes may be selected as sources for capsids of AAV viral vectors (DNase resistant viral particles) including, e.g., AAVI, AAV2, AAV6, AAV8, AAV9, AAVrh74, AAVrh10, AAV5, AAV7, AAVS3, AAVHSC, AAV2.7m8, AAV-LK03, AAV8/Olig001, AAV218, AAVhu37, AAV2tYF, AAVh1, AAVhu68, AAVrh.8, AAVrh9, AAV.PHP.B., AAV.PHP.eB, AAV.PHP.S, AAV/BBB, AAV-DJ, AAVr3.45, AAV-sh10, AAV2 (Y444F), AAV4, AAV-RPF2, AAV3b, AAVrh64R1, or variants of any of the known or mentioned AAVs or AAVs yet to be discovered. See, e.g., US Published Patent Application No. 2007-0036760-A1; US Published Patent Application No. 2009-0197338-A1; EP 1310571. See also, WO 2003/042397 (AAV7 and other simian AAV), U.S. Pat. Nos. 7,790,449 and 7,282,199 (AAV8), WO 2005/033321 and U.S. Pat. No. 7,906,111 (AAV9), and WO 2006/110689, and WO 2003/042397 (rh.10). Alternatively, a recombinant AAV based upon any of the recited AAVs, may be used as a source for the AAV capsid. These documents also describe other AAV which may be selected for generating AAV and are incorporated by reference. In some embodiments, an AAV cap for use in the viral vector can be generated by mutagenesis (i.e., by insertions, deletions, or substitutions) of one of the aforementioned AAV Caps or its encoding nucleic acid. In some embodiments, the AAV capsid is chimeric, comprising domains from two or three or four or more of the aforementioned AAV capsid proteins. In some embodiments, the AAV capsid is a mosaic of Vp1, Vp2, and Vp3 monomers from two or three different AAVs or recombinant AAVs. In some embodiments, an rAAV composition comprises more than one of the aforementioned Caps.

For packaging an expression cassette into virions, the ITRs are the only AAV components required in cis in the same construct as the gene. In one embodiment, the coding sequences for the replication (rep) and/or capsid (cap) are removed from the AAV genome and supplied in trans or by a packaging cell line in order to generate the AAV vector. For example, as described above, a pseudotyped AAV may contain ITRs from a source which differs from the source of the AAV capsid. Additionally or alternatively, a chimeric AAV capsid may be utilized. Still other AAV components may be selected. Sources of such AAV sequences are described herein and may also be isolated or obtained from academic, commercial, or public sources (e.g., the American Type Culture Collection, Manassas, Va.). Alternatively, the AAV sequences may be obtained through synthetic or other suitable means by reference to published sequences such as are available in the literature or in databases such as, e.g., GenBank, PubMed, or the like.

Methods for generating and isolating AAV viral vectors that can be used for delivery to a subject are known in the art. See, e.g., U.S. Pat. Nos. 7,790,449; 7,282,199; WO 2003/042397; WO 2005/033321, WO 2006/110689; and U.S. Pat. No. 7,588,772 B2]. In a one system, a producer cell line is transiently transfected with a construct that encodes the transgene flanked by ITRs and a construct(s) that encodes rep and cap. In a second system, a packaging cell line that stably supplies rep and cap is transiently transfected with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV—the required helper functions (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065. See generally, e.g., Grieger & Samulski, 2005, “Adeno-associated virus as a gene therapy vector: Vector development, production and clinical applications,” Adv. Biochem. Engin/Biotechnol. 99:119-145; Buning et al., 2008, “Recent developments in adeno-associated virus vector technology,” J. Gene Med. 10:717-733; and the references cited below, each of which is incorporated herein by reference in its entirety. The methods used to construct any embodiment of this invention are known to those with skill in nucleic acid manipulation and include genetic engineering, recombinant engineering, and synthetic techniques. See, e.g., Green and Sambrook et al, Molecular Cloning: A Laboratory Manual, Cold Spring Harbor Press, Cold Spring Harbor, N.Y. (2012). Similarly, methods of generating rAAV virions are well known and the selection of a suitable method is not a limitation on the present invention. See, e.g., K. Fisher et al, (1993) J. Virol., 70:520-532 and U.S. Pat. No. 5,478,745.

Optionally, the therapeutic peptide or fusion polypeptide described herein may be delivered via viral vectors other than rAAV. For example, other viral vectors that can be used herein include herpes simplex virus (HSV)-based vectors. HSV vectors deleted of one or more immediate early genes (IE) are advantageous because they are generally non-cytotoxic, persist in a state similar to latency in the target cell, and afford efficient target cell transduction. Recombinant HSV vectors can incorporate approximately 30 kb of heterologous nucleic acid.

Retroviruses, such as C-type retroviruses and lentiviruses, might also be used in the application. For example, retroviral vectors may be based on murine leukemia virus (MLV). See, e.g., Hu and Pathak, Pharmacol. Rev. 52:493-511, 2000 and Fong et al., Crit. Rev. Ther. Drug Carrier Syst. 17:1-60, 2000. MLV-based vectors may contain up to 8 kb of heterologous (therapeutic) DNA in place of the viral genes. The heterologous DNA may include a tissue-specific promoter and a nucleic acid encoding the therapeutic peptide. In methods of delivery to neural cells, it may also encode a ligand to a tissue specific receptor.

Additional retroviral vectors that might be used are replication-defective lentivirus-based vectors, including human immunodeficiency (HIV)-based vectors. See, e.g., Vigna and Naldini, J. Gene Med. 5:308-316, 2000 and Miyoshi et al., J. Virol. 72:8150-8157, 1998. Lentiviral vectors are advantageous in that they are capable of infecting both actively dividing and non-dividing cells.

Lentiviral vectors for use in the application may be derived from human and non-human (including SIV) lentiviruses. Examples of lentiviral vectors include nucleic acid sequences required for vector propagation as well as a tissue-specific promoter operably linked to a therapeutic peptide encoding nucleic acid. These former may include the viral LTRs, a primer binding site, a polypurine tract, att sites, and an encapsidation site.

In some aspects, a lentiviral vector can be employed. Lentiviruses have proven capable of transducing different types of CNS neurons (Azzouz et al., (2002) J Neurosci. 22:10302-12) and may be used in some embodiments because of their large cloning capacity.

A lentiviral vector may be packaged into any lentiviral capsid. The substitution of one particle protein with another from a different virus is referred to as “pseudotyping”. The vector capsid may contain viral envelope proteins from other viruses, including murine leukemia virus (MLV) or vesicular stomatitis virus (VSV). The use of the VSV G-protein yields a high vector titer and results in greater stability of the vector virus particles.

Alphavirus-based vectors, such as those made from semliki forest virus (SFV) and sindbis virus (SIN) might also be used in the application. Use of alphaviruses is described in Lundstrom, K., Intervirology 43:247-257, 2000 and Perri et al., Journal of Virology 74:9802-9807, 2000.

Recombinant, replication-defective alphavirus vectors are advantageous because they are capable of high-level heterologous (therapeutic) gene expression, and can infect a wide target cell range. Alphavirus replicons may be targeted to specific cell types by displaying on their virion surface a functional heterologous ligand or binding domain that would allow selective binding to target cells expressing a cognate binding partner. Alphavirus replicons may establish latency, and therefore long-term heterologous nucleic acid expression in a target cell. The replicons may also exhibit transient heterologous nucleic acid expression in the target cell.

In many of the viral vectors compatible with methods of the application, more than one promoter can be included in the vector to allow more than one heterologous gene to be expressed by the vector. Further, the vector can comprise a sequence, which encodes a signal peptide or other moiety, which facilitates expression of the therapeutic peptide from the target cell.

To combine advantageous properties of two viral vector systems, hybrid viral vectors may be used to deliver a nucleic acid encoding a therapeutic peptide to a target neuron, cell, or tissue. Standard techniques for the construction of hybrid vectors are well-known to those skilled in the art. Such techniques can be found, for example, in Sambrook, et al., In Molecular Cloning: A laboratory manual. Cold Spring Harbor, N.Y. or any number of laboratory manuals that discuss recombinant DNA technology. Double-stranded AAV genomes in adenoviral capsids containing a combination of AAV and adenoviral ITRs may be used to transduce cells. In another variation, an AAV vector may be placed into a “gutless”, “helper-dependent” or “high-capacity” adenoviral vector. Adenovirus/AAV hybrid vectors are discussed in Lieber et al., J. Virol. 73:9314-9324, 1999. Retrovirus/adenovirus hybrid vectors are discussed in Zheng et al., Nature Biotechnol. 18:176-186, 2000. Retroviral genomes contained within an adenovirus may integrate within the target cell genome and effect stable gene expression.

Other nucleotide sequence elements, which facilitate expression of the therapeutic peptide and cloning of the vector are further contemplated. For example, the presence of enhancers upstream of the promoter or terminators downstream of the coding region, for example, can facilitate expression.

In accordance with another embodiment, a tissue-specific promoter can be fused to nucleotides encoding the therapeutic peptides described herein. By fusing such tissue specific promoter within the adenoviral construct, transgene expression is limited to a particular tissue. The efficacy of gene expression and degree of specificity provided by tissue specific promoters can be determined, using the recombinant adenoviral system.

In addition to viral vector-based methods, non-viral methods may also be used to introduce a nucleic acid encoding a therapeutic peptide into a target cell. A review of non-viral methods of gene delivery is provided in Nishikawa and Huang, Human Gene Ther. 12:861-870, 2001. An example of a non-viral gene delivery method according to the application employs plasmid DNA to introduce a nucleic acid encoding a therapeutic peptide into a cell. Plasmid-based gene delivery methods are generally known in the art.

Synthetic gene transfer molecules can be designed to form multimolecular aggregates with plasmid DNA. These aggregates can be designed to bind to a target cell. Cationic amphiphiles, including lipopolyamines and cationic lipids, may be used to provide receptor-independent nucleic acid transfer into target cells.

In addition, preformed cationic liposomes or cationic lipids may be mixed with plasmid DNA to generate cell-transfecting complexes. Methods involving cationic lipid formulations are reviewed in Felgner et al., Ann. N.Y. Acad. Sci. 772:126-139, 1995 and Lasic and Templeton, Adv. Drug Delivery Rev. 20:221-266, 1996. For gene delivery, DNA may also be coupled to an amphipathic cationic peptide (Fominaya et al., J. Gene Med. 2:455-464, 2000).

Methods that involve both viral and non-viral based components may be used according to the application. For example, an Epstein Barr virus (EBV)-based plasmid for therapeutic gene delivery is described in Cui et al., Gene Therapy 8:1508-1513, 2001. Additionally, a method involving a DNA/ligand/polycationic adjunct coupled to an adenovirus is described in Curiel, D. T., Nat. Immun. 13:141-164, 1994.

Additionally, the nucleic acid encoding the therapeutic peptides of fusion polypeptide can be introduced into the target cell by transfecting the target cells using electroporation techniques. Electroporation techniques are well known and can be used to facilitate transfection of cells using plasmid DNA.

Vectors that encode the expression of the therapeutic peptides can be delivered in vivo to the target cell in the form of an injectable preparation containing pharmaceutically acceptable carrier, such as saline, as necessary. Other pharmaceutical carriers, formulations and dosages can also be used in accordance with the present application.

The therapeutic peptide can be expressed for any suitable length of time within the target cell, including transient expression and stable, long-term expression.

The therapeutic peptide, fusion polypeptides, or vectors described herein may be formulated with one or more pharmaceutically acceptable carrier or excipients to provide a pharmaceutical composition. The therapeutic peptide, fusion polypeptides, or vectors described herein may be combined with a pharmaceutically acceptable buffer, and the pH adjusted to provide acceptable stability, and a pH acceptable for administration such as parenteral administration. Optionally, one or more pharmaceutically acceptable anti-microbial agents may be added. Meta-cresol and phenol are preferred pharmaceutically acceptable microbial agents. One or more pharmaceutically acceptable salts may be added to adjust the ionic strength or tonicity. One or more excipients may be added to further adjust the isotonicity of the formulation. Glycerin is an example of an isotonicity-adjusting excipient. Pharmaceutically acceptable means suitable for administration to a human or other animal does not contain toxic elements or undesirable contaminants and does not interfere with the activity of the active compounds therein.

In some embodiments, the therapeutic peptide or fusion polypeptides, described herein may be formulated as a solution formulation or as a lyophilized powder that can be reconstituted with an appropriate diluent. A lyophilized dosage form is one in which the therapeutic peptide or fusion polypeptide is stable, with or without buffering capacity to maintain the pH of the solution over the intended in-use shelf-life of the reconstituted product. It is preferable that the solution comprising the therapeutic peptide or fusion polypeptide described herein before lyophilization be substantially isotonic to enable formation of isotonic solutions after reconstitution.

A pharmaceutically-acceptable salt form of the therapeutic peptide or fusion polypeptides described herein can also be provided. Acids commonly employed to form acid addition salts are inorganic acids such as hydrochloric acid, hydrobromic acid, hydriodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Preferred acid addition salts are those formed with mineral acids such as hydrochloric acid and hydrobromic acid.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, and the like.

The therapeutic peptide, fusion polypeptide, or vector expressing the therapeutic peptide or fusion polypeptide can be delivered to a subject by any suitable route, including, for example, local and/or systemic administration. Systemic administration can include, for example, parenteral administration, such as intramuscular, intravenous, intraarticular, intraarterial, intrathecal, subcutaneous, or intraperitoneal administration. The agent can also be administered orally, transdermally, topically, by inhalation (e.g., intrabronchial, intranasal, oral inhalation or intranasal drops) or rectally. In some embodiments, the therapeutic peptide, fusion polypeptide, or vector can be administered to the subject via intravenous administration using an infusion pump to deliver daily, weekly, or doses of the therapeutic agent.

Pharmaceutically acceptable formulations of the therapeutic agent can be suspended in aqueous vehicles and introduced through conventional hypodermic needles or using infusion pumps.

For injection, the therapeutic peptide, fusion polypeptide, or vector described herein can be formulated in liquid solutions, typically in physiologically compatible buffers such as Hank's solution or Ringer's solution. In addition, the therapeutic agent may be formulated in solid form and re-dissolved or suspended immediately prior to use. Lyophilized forms are also included. The injection can be, for example, in the form of a bolus injection or continuous infusion (such as using infusion pumps) of the therapeutic agent.

In the methods of treatment disclosed herein, a therapeutically effective amount of the therapeutic peptide is administered to the subject to treat or prevent one or more of insulin resistance, obesity, an obesity related condition, diabetes, a metabolic disease or disorder, such as metabolic syndrome, and being overweight in a subject in need thereof.

It will be appreciated that the amount, volume, concentration, and/or dosage of the therapeutic peptide, fusion polypeptide, or vector that is administered to any one animal or human depends on many factors, including the subject's size, body surface area, age, the particular composition to be administered, sex, time and route of administration, general health, and other drugs being administered concurrently. Specific variations of the above noted amounts, volumes, concentrations, and/or dosages of the therapeutic peptide, fusion polypeptide, or vector can readily be determined by one skilled in the art using the experimental methods described below.

Doses may be in the range of 0.01 to 10 mg/kg body weight. In an embodiment, the doses may be in the range of 0.05 to 5 mg/kg body weight. In another embodiment, the doses may be in the range of 0.01 to 1 mg/kg body weight. In still another embodiment, the doses may be in the range of 0.05 to 0.5 mg/kg body weight. In still another embodiment, the doses may be in the range of 0.05 to 1 mg/kg body weight.

The therapeutic peptide, fusion polypeptide, or vector can be administered at an interval of one week or greater. Depending on the disease being treated, it may be necessary to administer the therapeutic peptide, fusion polypeptide, or vector described herein more frequently than the one week interval, such as two to three time per week.

For example, in accordance with embodiments, the doses may be administered at an interval of 1 week or greater. In one embodiment, the doses may be administered at an interval of 2 weeks or greater. In another embodiment, the doses may be administered at an interval of 3 weeks or greater. In still another embodiment, the doses may be administered at an interval of 3 days, 4 days, 5 days, 6 days, 7 days, 8 days, 9 days, 10 days, 15 days, 20 days, 30 days, 40 days, or greater. In another embodiment, the doses may be administered at a frequency of once a week, twice a week, once every other week, or twice per month, three times per month, and the like.

In an aspect, the method comprises administering the therapeutic peptide or fusion polypeptide at a dose of from about 0.01 mg/kg to about 10 mg/kg, about 0.02 mg/kg to about 10 mg/kg, from about 0.03 mg/kg to about 10 mg/kg, about 0.04 mg/kg to about 10 mg/kg, from about 0.05 mg/kg to about 10 mg/kg, about 0.06 mg/kg to about 10 mg/kg, from about 0.07 mg/kg to about 10 mg/kg, from about 0.08 mg/kg to about 10 mg/kg, from about 0.09 mg/kg to about 10 mg/kg, from about 0.1 mg/kg to about 10 mg/kg, from about 0.15 mg/kg to about 10 mg/kg, from about 0.2 mg/kg to about 10 mg/kg, from about 0.25 mg/kg to about 10 mg/kg, from about 0.3 mg/kg to about 10 mg/kg, from about 0.35 mg/kg to about 10 mg/kg, from about 0.4 mg/kg to about 10 mg/kg, from about 0.45 mg/kg to about 10 mg/kg, from about 0.5 mg/kg to about 10 mg/kg, from about 0.55 mg/kg to about 10 mg/kg, from about 0.6 mg/kg to about 10 mg/kg, from about 0.65 mg/kg to about 10 mg/kg, from about 0.7 mg/kg to about 10 mg/kg, from about 0.75 mg/kg to about 10 mg/kg, from about 0.8 mg/kg to about 10 mg/kg, from about 0.85 mg/kg to about 10 mg/kg, from about 0.9 mg/kg to about 10 mg/kg, from about 0.95 mg/kg to about 10 mg/kg, from about 1 mg/kg to about 10 mg/kg, from about 1.1 mg/kg to about 10 mg/kg, from about 1.2 mg/kg to about 10 mg/kg, from about 1.3 mg/kg to about 10 mg/kg, from about 1.4 mg/kg to about 10 mg/kg, from about 1.5 mg/kg to about 10 mg/kg, from about 1.6 mg/kg to about 10 mg/kg, from about 1.7 mg/kg to about 10 mg/kg, from about 1.8 mg/kg to about 10 mg/kg, from about 1.9 mg/kg to about 10 mg/kg, from about 2 mg/kg to about 10 mg/kg, from about 2.1 mg/kg to about 10 mg/kg, from about 2.2 mg/kg to about 10 mg/kg, from about 2.3 mg/kg to about 10 mg/kg, from about 2.4 mg/kg to about 10 mg/kg, from about 2.5 mg/kg to about 10 mg/kg, from about 2.6 mg/kg to about 1.0 mg/kg, from about 2.7 mg/kg to about 10 mg/kg, from about 2.8 mg/kg to about 10 mg/kg, from about 2.9 mg/kg to about 10 mg/kg, from about 3 mg/kg to about 10 mg/kg, from about 3.1 mg/kg to about 10 mg/kg, from about 3.2 mg/kg to about 10 mg/kg, from about 3.3 mg/kg to about 10 mg/kg, from about 3.4 mg/kg to about 10 mg/kg, from about 3.5 mg/kg to about 10 mg/kg, from about 3.6 mg/kg to about 10 mg/kg, from about 3.7 mg/kg to about 10 mg/kg, from about 3.8 mg/kg to about 10 mg/kg, from about 3.9 mg/kg to about 10 mg/kg, or from about 4 mg/kg to about 10 mg/kg, at an interval of 1 week or greater, at an interval of 2 weeks or greater, at an interval of 3 weeks or greater, or at an interval of 4 weeks or greater. In embodiments, the upper limit of the above ranges may be about 5 mg/kg. In another embodiments, the doses may be administered at an interval of 3 days, 4 days, 5 days, 6 days, 7 days, 8 days, 9 days, 10 days, 15 days, 20 days, 30 days, 40 days, or greater. In another embodiment, the doses may be administered at a frequency of once a week, twice a week, once every other week, or twice per month, three times per month, and the like.

In another embodiment, the therapeutic peptide or fusion polypeptide may be administered at a dose of from about 0.01 mg/kg to about 1 mg/kg, about 0.02 mg/kg to about 1 mg/kg, from about 0.03 mg/kg to about 1 mg/kg, about 0.04 mg/kg to about 1 mg/kg, from about 0.05 mg/kg to about 1 mg/kg, about 0.06 mg/kg to about 1 mg/kg, from about 0.07 mg/kg to about 1 mg/kg, from about 0.08 mg/kg to about 1 mg/kg, from about 0.09 mg/kg to about 1 mg/kg, from about 0.1 mg/kg to about 1 mg/kg, from about 0.16 mg/kg to about 1 mg/kg, from about 0.2 mg/kg to about 1 mg/kg, from about 0.24 mg/kg to about 1 mg/kg, from about 0.3 mg/kg to about 1 mg/kg, from about 0.35 mg/kg to about 1 mg/kg, from about 0.4 mg/kg to about 1 mg/kg, from about 0.45 mg/kg to about 1 mg/kg, from about 0.5 mg/kg to about 1 mg/kg, from about 0.55 mg/kg to about 1 mg/kg, from about 0.6 mg/kg to about 1 mg/kg, from about 0.65 mg/kg to about 1 mg/kg, from about 0.7 mg/kg to about 1 mg/kg, from about 0.75 mg/kg to about 1 mg/kg, from about 0.8 mg/kg to about 1 mg/kg, from about 0.85 mg/kg to about 1 mg/kg, from about 0.9 mg/kg to about 1 mg/kg, from about 0.95 mg/kg to about 1 mg/kg, at an interval of 1 week or greater, at an interval of 2 weeks or greater, at an interval of 3 weeks or greater, or at an interval of 4 weeks or greater. In still another embodiment, the doses may be administered at an interval of 3 days, 4 days, 5 days, 6 days, 7 days, 8 days, 9 days, 10 days, 15 days, 20 days, 30 days, 40 days, or greater. In another embodiment, the doses may be administered at a frequency of once a week, twice a week, once every other week, or twice per month, three times per month, and the like.

In another aspect, the method comprises administering the therapeutic peptide or fusion polypeptide at a dose of from about 0.1 mg/kg to about 5 mg/kg, from about 0.2 mg/kg to about 5 mg/kg, from about 0.3 mg/kg to about 5 mg/kg, from about 0.4 mg/kg to about 5 mg/kg, from about 0.5 mg/kg to about 5 mg/kg, from about 0.6 mg/kg to about 5 mg/kg, from about 0.7 mg/kg to about 5 mg/kg, from about 0.8 mg/kg to about 5 mg/kg, from about 0.9 mg/kg to about 5 mg/kg, from about 1 mg/kg to about 5 mg/kg, from about 1.1 mg/kg to about 5 mg/kg, from about 1.2 mg/kg to about 5 mg/kg, from about 1.3 mg/kg to about 5 mg/kg, from about 1.4 mg/kg to about 5 mg/kg, from about 1.5 mg/kg to about 5 mg/kg, from about 1.6 mg/kg to about 5 mg/kg, from about 1.7 mg/kg to about 5 mg/kg, from about 1.8 mg/kg to about 5 mg/kg, from about 1.9 mg/kg to about 5 mg/kg, from about 2 mg/kg to about 5 mg/kg, from about 2.1 mg/kg to about 5 mg/kg, from about 2.2 mg/kg to about 5 mg/kg, from about 2.3 mg/kg to about 5 mg/kg, from about 2.4 mg/kg to about 5 mg/kg, from about 2.5 mg/kg to about 5 mg/kg, from about 2.6 mg/kg to about 5 mg/kg, from about 2.7 mg/kg to about 5 mg/kg, from about 2.8 mg/kg to about 5 mg/kg, from about 2.9 mg/kg to about 5 mg/kg, from about 3 mg/kg to about 5 mg/kg, from about 3.1 mg/kg to about 5 mg/kg, from about 3.2 mg/kg to about 5 mg/kg, from about 3.3 mg/kg to about 5 mg/kg, from about 3.4 mg/kg to about 5 mg/kg, from about 3.5 mg/kg to about 5 mg/kg, from about 3.6 mg/kg to about 5 mg/kg, from about 3.7 mg/kg to about 5 mg/kg, from about 3.8 mg/kg to about 5 mg/kg, from about 3.9 mg/kg to about 5 mg/kg, or from about 4 mg/kg to about 5 mg/kg, at an interval of 3 days, 4 days, 5 days, 6 days, 7 days, 8 days, 9 days, 10 days, 15 days, 20 days, 30 days, 40 days, or greater. In another embodiment, the doses may be administered at a frequency of once a week, twice a week, once every two weeks, once per month, twice per month, three times per month, and the like.

In some embodiments, the therapeutic peptide or fusion polypeptide is administered at a dose of 0.05 mg/kg, 0.06 mg/kg, 0.07 mg/kg, 0.08 mg/kg, 0.09 mg/kg, 0.1 mg/kg, 0.11 mg/kg, 0.12 mg/kg, 0.13 mg/kg, 0.14 mg/kg, 0.15 mg/kg, 0.16 mg/kg, 0.17 mg/kg, 0.18 mg/kg, 0.19 mg/kg, 0.2 mg/kg, 0.21 mg/kg, 0.22 mg/kg, 0.23 mg/kg, 0.24 mg/kg, 0.25 mg/kg, 0.26 mg/kg, 0.27 mg/kg, 0.28 mg/kg, 0.29 mg/kg, or 3 mg/kg at an interval of 1 week or two weeks. It should be understood that the two weeks interval schedule may be replaced with a frequency of every other week.

In another embodiment, the therapeutic peptide or fusion polypeptide is administered at a dose of 0.05 mg/kg, 0.06 mg/kg, 0.07 mg/kg, 0.08 mg/kg, 0.09 mg/kg, 0.1 mg/kg, 0.11 mg/kg, 0.12 mg/kg, 0.13 mg/kg, 0.14 mg/kg, 0.15 mg/kg, 0.16 mg/kg, 0.17 mg/kg, 0.18 mg/kg, 0.19 mg/kg, 0.2 mg/kg at an interval of 1 week or 10 days.

In another embodiment, the therapeutic peptide or fusion polypeptide is administered at a dose of 0.1 mg/kg, 0.11 mg/kg, 0.12 mg/kg, 0.13 mg/kg, 0.14 mg/kg, 0.15 mg/kg, 0.16 mg/kg, 0.17 mg/kg, 0.18 mg/kg, 0.19 mg/kg, 0.2 mg/kg, 0.21 mg/kg, 0.22 mg/kg, 0.23 mg/kg, 0.24 mg/kg, 0.25 mg/kg, 0.26 mg/kg, 0.27 mg/kg, 0.28 mg/kg, 0.29 mg/kg, or 3 mg/kg at an interval of 2 weeks, or with a frequency of once every other week, twice per month, or three times per month.

In other embodiments, a pharmaceutically acceptable formulation used to administer the therapeutic peptide, fusion polypeptide, or vector can also be formulated to provide sustained delivery of the active compound to a subject. For example, the formulation may deliver the active compound for at least one, two, three, or four weeks, inclusive, following initial administration to the subject. For example, a subject to be treated in accordance with the method described herein can be treated with the therapeutic agent for at least 30 days (either by repeated administration or by use of a sustained delivery system, or both).

Approaches for sustained delivery include use of a polymeric capsule, a minipump to deliver the formulation, a biodegradable implant, or implanted transgenic autologous cells (see U.S. Pat. No. 6,214,622). Implantable infusion pump systems (e.g., INFUSAID pumps (Towanda, PA)); see Zierski et al., 1988; Kanoff, 1994) and osmotic pumps (sold by Alza Corporation) are available commercially and otherwise known in the art. Another mode of administration is via an implantable, externally programmable infusion pump. Infusion pump systems and reservoir systems are also described in, e.g., U.S. Pat. Nos. 5,368,562 and 4,731,058.

Vectors encoding the therapeutic peptides or fusion polypeptides can often be administered less frequently than other types of therapeutics. For example, an effective amount of such a vector can range from about 0.01 mg/kg to about 5 or 10 mg/kg, inclusive; administered daily, weekly, biweekly, monthly or less frequently.

The pharmaceutical compositions can be administered to any subject that can experience the beneficial effects of inhibition of asprosin-mediated orexigenesis and/or glucogenesis. Foremost among such animals are humans, although the present invention is not intended to be so limited.

The therapeutic peptide, fusion polypeptide, or vector expressing the therapeutic peptide or fusion polypeptide described herein can be used in methods and compositions for treating or preventing one or more of asprosin-mediated orexigenesis and/or glucogenesis related disease or disorders. Such diseases or disorders can include, for example, insulin resistance, obesity, an obesity related condition, diabetes, a metabolic disease or disorder, such as metabolic syndrome, and/or decreasing weight in an individual in need of weight loss. Other metabolic and/or endocrine related diseases or disorders that can be treated diseases include types 1 and 2 diabetes, pancreatitis, dyslipidemia, nonalcoholic fatty liver disease, nonalcoholic steatohepatitis, hyperinsulinemia, glucose intolerance, hyperglycemia, acute myocardial infarction, hypertension, cardiovascular disease, atherosclerosis, peripheral arterial disease, stroke, heart failure, coronary heart disease, nephropathy, diabetic complication, neuropathy, gastroparesis, and symptoms associated with the severe inactivation mutations of insulin receptor.

In some embodiment the individual to be treated may be in need of a decrease in adipose mass. The individual may be in need of weight loss for a variety of reasons, including because of a medical condition or state or for another reason. In cases wherein the individual is in need of weight loss because of a medical condition, the medical condition may or may not be a genetic condition and may or may not be an inherited condition. The cause of being in need of weight loss may be from genetics, metabolism, and/or illness. In specific embodiments, the medical condition has being overweight or obese as a symptom. In some cases, the symptom of being overweight or obese is present in all individuals with the medical condition, although it may be present in less than all individuals with the medical condition. The symptom of being overweight or obese may be because of a defect in pathways related to adipose metabolic regulation, fat storage, and inflammatory processes, although in some cases being overweight or obese is not directly related to adipose metabolic regulation, fat storage, and inflammatory processes. The individual may be overweight or obese because of diabetes; hypothyroidism; metabolic disorders, including metabolic syndrome; medication side effects; alcoholism; eating disorder; insufficient sleep; limited physical exercise; sedentary lifestyle; poor nutrition; addiction cessation; and/or stress; although in some embodiments such conditions are the result of being overweight or obese.

In some methods, an individual is in need of modulation of hepatic glucose release; such embodiments may modulate (such as activate) pathways that control rapid glucose release into the circulation. In particular embodiments, an individual has a defect in glucose control and is determined to need an improvement in such defect. In specific embodiments, the defect in glucose control is that there is an excessive amount of glucose in the blood of the individual. In particular embodiments, an individual has diabetes or is pre-diabetic and may or may not also be overweight or obese. The individual is provided an effective amount of one or more therapeutic peptides or fusion polypeptides to improve blood glucose control, in specific embodiments, including to reduce the level of excessive blood glucose. Such treatment is provided to the diabetic or pre-diabetic individual and an improvement in blood glucose control occurs. The decrease in blood glucose level may or may not be too normal blood glucose levels. In particular embodiments, in addition to an improvement in blood glucose control, one or more symptoms of diabetes or pre-diabetes is improved upon administration of one or more therapeutic peptides or fusion polypeptides described herein. Some methods of the disclosure treat insulin resistance, such as by reducing levels of asprosin mediated orexigenesis and/or glucogenesis by sequestering plasma asprosin using the therapeutic peptides or fusion polypeptides described herein. For pre-diabetic individuals, the onset of diabetes is prevented upon use of one or more therapeutic peptides or fusion polypeptides described herein. For insulin-resistant individuals, inhibiting asprosin mediated orexigenesis and/or glucogenesis results in restoration or improvement of insulin sensitivity, resulting in better glucose clearance, in specific embodiments.

In specific embodiments, an individual in need of weight loss is overweight (BMI between 25 and 29) or obese (BMI of 30 or more). The individual that is subjected to methods and compositions of the disclosure may first be identified by a medical practitioner as being in need of weight loss, and a therapeutic composition comprising one or more therapeutic peptides or fusion polypeptides described herein may be delivered to the individual for the specific purpose of decreasing weight.

In some embodiments an individual is determined to be in need of weight loss, such as by measuring their weight and/or by measuring their BMI and/or having an MRI and/or DEXA scan for assessment of adipose mass. The individual may be known to be in need of weight loss or suspected of being in need of weight loss or at risk for being in need of weight loss. An individual may determine themselves that they are in need of weight loss and/or it may be determined by a suitable medical practitioner.

Once the individual is known to be in need of weight loss or known to be at risk or susceptible to being in need of weight loss, they may be given a suitable and effective amount of the therapeutic peptide, fusion polypeptide, or vector described herein. In specific embodiments, the therapeutic peptide, fusion polypeptide, or vector are provided to the individual, such as in a composition or in multiple compositions. A composition comprising the of the therapeutic peptide, fusion polypeptide, or vector may be specifically formulated for a therapeutic application.

The individual may or may not be monitored by a medical practitioner during the course of the therapeutic peptide or fusion polypeptide regimen. The individual may cease to take the therapeutic peptide or fusion polypeptide once a desirable weight is achieved and may resume taking the therapeutic peptides or fusion polypeptides if the individual becomes in need of losing weight at a later point in time. In the event that an individual exceeds a suitable amount of the therapeutic peptides or fusion polypeptides such that too much weight is lost, the individual may increase their weight by any suitable means, including by increasing caloric intake.

In one embodiment, the subject to be treated has an increased appetite, hunger or craving for sweet or fat food. This may be related to e.g., stress, quit of smoking, pregnancy, premenstrual tension, or it can be ascribed physiological problems or diseases, such as binge eating, compulsive eating habits and Seasonal Affective Disorder.

Binge eating disorder (BED) is a fairly new diagnosable disorder—see e.g., Int. J. Obesity, 2002, 26, 299-307 and Curr. Opin. Psychiatry, 17, 43-48, 2004. BED is characterized by binge eating episodes as is bulimia nervosa (BN). However, subjects with BED do not, contrary to patients with BN, engage in compensatory behaviors, such as e.g., self-induced vomiting, excessive exercise, and misuse of laxatives, diuretics or enemas. Studies have shown that 1-3% of the general population suffer from BED, whereas a higher prevalence (up to 25-30%) have been reported for obese patients [Int. J. Obesity, 2002, 26, 299-307]. These numbers show that BED subjects may or may not be obese, and that obese patients may or may not have BED, i.e., that the cause of the obesity is BED. However, a proportion of subjects with BED eventually becomes obese due to the excess calorie intake. Laboratory studies have shown that BED patients consumed more dessert and snack (rich in fat and poor in proteins) than did an obese control group [Int. J. Obesity, 2002, 26, 299-307], and the therapeutic peptides or fusion polypeptides described herein are thus believed to be suited for treatment of BED. In one embodiment, a method or treating BED in a subject includes administering to the subject an effective amount of the therapeutic peptides or fusion polypeptides. In particular, said subject is obese.

Bulimia nervosa (BN) is characterized by the same binge eating episodes as is BED, however, BN is, however, also characterized by the above mentioned compensatory behavior. A proportion of subjects with BN will eventually become obese to the extent that the compensatory behavior cannot fully compensate the excess calorie intake. Studies have compared binges of patients with BN and with BED concluding that binges in subjects with BN were higher in carbohydrates and sugar content than those of subjects with BED. No difference was, however, found in the number of consumed calories [Int. J. Obesity, 2002, 26, 299-307]. The compositions and methods described herein are believed to be suited for the treatment of BN.

The invention is further illustrated by the following example, which is not intended to limit the scope of the claims.

### Example

Asprosin is a fasting-induced glucogenic and centrally-acting orexigenic hormone. We have now identified the receptor that asprosin engages to activate the central appetite circuitry. It is Protein Tyrosine Phosphatase Receptor δ (Ptprd), a hitherto orphan, membrane bound receptor that is highly expressed in AgRP neurons (the key hypothalamic cell type responsible for asprosin-mediated appetite stimulation). Asprosin functions as a high-affinity Ptprd ligand and regulates its activity in a cell autonomous manner. The genetic ablation of Ptprd results in a strong loss of appetite, leanness, and an inability to respond to the orexigenic effects of asprosin. Ablation of Ptprd specifically in AgRP neurons causes resistance to diet induced obesity. The soluble ligand binding domain of Ptprd when introduced into the circulation suppresses appetite and blood glucose levels by sequestering plasma asprosin. Identification of Ptprd as the orexigenic asprosin-receptor creates a new avenue for development of anti-obesity therapeutics.

### Experimental Model and Subject Details

### Mouse Models

Wildtype C57BL/6 mice (WT mice; Jackson Laboratory, JAX: 000664), diet induced obese mice (DIO mice; JAX: 380050), Rosa26-Flipase⁺ mice (Jackson Laboratory, JAX: 003946), AgRP-RES-Cre (C57BL/6-Agrptm1(cre)Low1; Jackson Laboratory, JAX: 012899), Rosa26-LSL-tdTOMATO (JAX: 007905), Rosa26-Ribotag (Jackson Laboratory, JAX: 029977) were purchased from The Jackson Laboratory. Two different strains of Ptprd mice were used in this work and were maintained as heterozygous. B6;129-Ptprd^{<tm1Yiw>} mice were purchased from the RIKEN BioResource Center. Mice with Ptprd cKO potential (C57BL/6N-A<tm1Brd>Ptprd<tm2a(KOMP)Wtsi>/WtsiOr1; MEXY mice) was purchased from Wellcome Trust Sanger Institute and crossed to Flpase⁺ mice to remove the neomycin selection cassette and LacZ reporter, thereby making a conditional allele. Thereafter, homozygous conditionally ready floxed mice (Ptprd tm2c(KOMP)Wtsi) were mated with AgRP-IRES-Cre (C57BL/6-Agrptm1(cre)Low1) to create AgRP neuron specific knock-out (KO) of Ptprd. For cKO of Ptprd in adult mice, CRISPR-cas9 mediated unilateral and bilateral KO was done in AgRP-IRES-Cre (C57BL/6-Agrptm1(cre)Low1) mice.

Mice were housed in microventilators on a 12-hour light cycle, and were fed normal chow (5V5R, Lab Supply), dustless pellet diet (F0173, Bio-Serv) or high fat diet (60% calories from fat, TD.06414, Envigo Teklad). Animal housing, husbandry, and euthanasia were conducted under animal protocols approved by the Case Western Reserve University Institutional Animal Care and Use Committee (protocol #2018-0042).

### Postnatal Knockdown of Ptprd in AgRP Neurons

CRISPR/Cas9 approach was used for unilateral and bilateral disruption of Ptprd selectively in AgRP neurons. Briefly, targeting efficiency of sgRNA against Ptprd was tested and confirmed with TIDE analysis before being cloned into AAV-ITR-U6-sgRNA plasmid.

To fully ablate the function of Ptprd selectively in AgRP neurons, a bilateral knockout of Ptprd was done in AgRP-IRES-Cre male and female mice (12 weeks old). AgRP-IRES-Cre mice received bilateral stereotaxic injections of AAV-FLEX-saCas9 (Vector Biolabs, #7122) and AAV-Ptprd/sgRNA-FLEX-mCherry in the arcuate nucleus of the hypothalamus (ARC). Wild type mice received the same viruses and AgRP-IRES-Cre mice received bilateral AAV-Ptprd/sgRNA-FLEX-mCherry and the AAV-mCherry (no Cas9) were served as two groups of control mice. Seven weeks post injection, mice were subjected to high fat diet for the next 5 weeks. Mice were monitored for changes in body weight and food intake throughout the experiment.

For unilateral knockdown of Ptprd, AgRP-IRES-Cre male mice (10-12 weeks old) received stereotaxic injections of AAV-FLEX-saCas9 (Vector Biolabs, #7122) with AAV-Ptprd/sgRNA-FLEX-GFP on one side and AAV-Ptprd/sgRNA-FLEX-GFP with AAV-mCherry (no Cas9) virus on the other side in the ARC. Six weeks after the stereotaxic injections, electrophysiology recordings in GFP-labeled AgRP neurons from each side of ARC (control vs. Ptprd-KO side) was performed.

### Cell Culture

HEK293T cells were maintained at 37° C. and 5% CO₂ in DMEM, containing 10% FBS (HyClone) and 100 mg ml-1 penicillin-streptomycin. In general, siRNA was transfected using transfection reagent (Dharmacon, T-2001-02) following manufacturer's protocol, and asprosin was overexpressed using Ad5-Asprosin or Asprosin coding mammalian expression plasmid with Ad5-empty or CMV6-Entry-empty as controls. For luciferase readout, HEK293T cells were transduced with 4xM67 pTATA-TK-Luc (Addgene; 8688) plasmid. HEK293T cell transfection, lysis and luciferase readouts were done with Fugene HD transfection reagent (Promega; E2312), Reporter Lysis 5× Buffer (Promega, E3971) and Luciferase Assay Reagent (Promega, E1483), respectively, using standard manufacturer's protocol.

### Immunoprecipitation and Mass Spectrometry

Cerebella were dissected, chopped into small pieces, and pooled from 5 adult mice and placed in 5 ml of DMEM media at room temperature. 300 μg of recombinant asprosin or GFP was added to each sample and incubated at room temperature for 10 min with rotation. Formaldehyde was added to a final concentration of 0.5% and samples were incubated at room temperature for 10 minutes with rotation. Crosslinking was quenched with addition of 125 mM glycine. Samples were incubated for another 5 minutes at room temperature with rotation. Cerebellar tissue was centrifuged at 2000 rpm for 5 minutes to collect tissue and then washed 3× with cold PBS. Crosslinked cerebellar tissue was then lysed with 3 times packed tissue volumes of NETN (170 mM NaCl, 1 mM EDTA, 50 mM Tris, and 0.5% NP40) by sonication (Sonics $ Materials, Inc., CT) with 25amp for total 3 min operation with 60 s rest every 30 sec. After ultracentrifugation (100 kg, 20 min, 4° C.), supernatant was pre-cleared with protein A beads. Pre-cleared supernatant was incubated with anti-asprosin antibody and antibody-protein complex were pulled down by protein A bead. Immunoprecipitated proteins complex were extracted from bead by boiling in laemmli buffer and separated by SDS-PAGE. After in-gel digestion by trypsin, peptides recovered from gel was analyzed with LC-MS/MS (Ultimate 3000 LC coupled with Orbitrap Fusion™ Tribrid™ ) based on a previous study. Search of the obtained mass spectrum was done in Proteome Discoverer 1.4 interface with the Mascot algorithm 2.4. In order to quantify asprosin and Ptprd, searched data and raw data was imported into Skyline proteomics application (MacCoss lab, Seattle, WA) and the AUC of unique peptides of each gene were calculated. Four peptides were used for each gene including 2 miscleavages without any static or dynamic modification.

For assessment of recombinant receptor-ligand interaction, 1 μg of recombinant (r) GST-Asprosin and rPTPRD-his protein (Creative BioMart, PTPRD-36H) were mixed and incubated at room temperature with rotation in IP Lysis Buffer (ThermoFisher Scientific, 87787) supplemented with 1X Halt protease inhibitor cocktail (ThermoFisher Scientific, 87786). Aliquots of this suspension were incubated with rabbit anti-PTPRD (ABclonal, A15713), mouse anti-asprosin, mouse IgG (Southern Biotech, 0107-01) or Rabbit IgG (Southern Biotech, 0111-01) for two hours. Thereafter, protein-antibody samples were loaded onto Protein G magnetic beads (ThermoFisher Scientific, 88847) and incubated for 1 hour with rotation. Elute from magnetic beads was subjected to western blot analysis of asprosin and PTPRD.

### Asprosin-PTPRD Affinity Assessment

### Surface Plasmon Resonance

Surface plasmon resonance (SPR) studies were performed using Biacore T200 with PTPRD (Acro Biosystems, PTD-H52H9) covalently immobilized on an S series CM5 sensor chip via amine coupling. To avoid protein aggregation, caspase-1 used in SPR experiments were purified by gel filtration in a buffer containing 25 mM Tris-HCl (pH 8.0), 100 mM NaCl and 5 mM DTT, stored at 4° C., and used within 48 hr with minimum concentration process. Asprosin was injected in a series of concentrations from 1 M to 7.8125 nM was injected at 30 μl/min over the sensor chip at room temperature. Sensorgram traces subtracted with the reference and zero-concentration traces were analyzed using BIA Evaluation software (GE Healthcare Bio-Sciences). Nonlinear steady-state analysis was performed to compute K_{d}.

### Microscale Thermophoresis

The microscale thermophoresis (MST) assay was performed with the Monolith NT.115 from NanoTemper Technologies. PTPRD (Acro Biosystems, PTD-H52H9) was fluorescently labeled according to the manufacturer's protocol (His-Tag Labeling Kit RED-tris-NTA, NanoTemper). A solution of unlabeled asprosin was serially diluted according to the manufacturer's protocol. After labeled PTPRD and unlabeled asprosin were mixed and incubated at room temperature for 30 minutes, samples were loaded into glass capillaries (Monolith NT.115 Capillaries, NanoTemper). Measurements were carried out according to manufacturer's protocol: 40% power and 40% MST power. Assays were repeated three times and K_{D}'s were calculated using the NanoTemper analysis software.

### Biometric Layer Interference

Binding affinity of asprosin to PTPRD was measured on Pall ForteBio's Octet RED96 system. The PTPRD protein (Creative BioMart, PTPRD-36H) was labeled with Biotin using EZ-Link-Sulfo-NHS-Biotin (ThermoFisher Scientific, 21217) and desalted by Zeba™ Spin Desalting Columns. The biotin-labeled PTPRD (20 μg/ml) was loaded on the super streptavidin (SSA) biosensors for 300 seconds. Following 20 seconds of baseline in kinetics buffer, the loaded biosensors were dipped into a series of 3-fold diluted human asprosin (1.23-900 nM) for 300 seconds to record association kinetics and then dipped into a kinetic buffer for 600 seconds to record dissociation kinetics. Kinetic buffer without asprosin was set to correct the background. For fitting of K_{D} value, ForteBio's data analysis software was used to fit the curve by a 1:1 binding model and the global fitting method was applied.

### RNA Scope Analysis of Ptprd and Olfr734

Mice were anesthetized and perfused transcardially with 0.9% saline followed by 10% formalin. Brains were removed and post fixed in 10% formalin for 16 h at 4° C. and cryoprotected in 30% sucrose for 48 hours. Brains were frozen, sectioned at 14 μm using the cryostat, washed in DEPC-treated phosphate buffered saline for 10 min. Sections were mounted on DEPC-treated charged slides, dried for 0.5 hour at room temperature and stored at −80° C. On the day of the RNAScope assay, the slides were thawed and slides were rinsed 2 times in PBS 1X and placed in an oven for 30 min at 60° C. After that, slides were post fixed in 10% formalin for 15 minutes at 4° C. Slides were then gradually dehydrated in ethanol (50, 70 and 100%, 5 min each) and underwent target retrieval for 5 minutes at 100° C. Slides were incubated in protease III (#322337, ACDBio) for 30 minutes at 40° C. Slides were then rinsed in distilled water and incubated in RNAScope probes for AgRP (Mm-AgRP; #400711, ACDBio) and Olfr734 (Mm-Olfr734-C3; #878653-C3, ACDBio) or Prprd (Mm-Ptprd-C2; #474651-C2, ACDBio,) for 2 hours at 40° C. Sections were then processed using the RNAScope Fluorescent Multiplex Detection Reagents (#320851, ACDBio) according to the manufacturer instructions. Slides were cover-slipped and analyzed using a fluorescence microscope.

### AgRP Neuron Labelling and Electrophysiology

AgRP neuron labeling, and electrophysiology experiments were performed as previously described. Briefly, to identify AgRP neurons, we crossed the Rosa26-LSL-tdTOMATO mice with AgRP-IRES-Cre mice to generate AgRP-IRES-Cre/Rosa26-LSL-tdTOMATO mice, which express tdTOMATO selectively in AgRP neurons. The entire brains of AgRP-IRES-Cre/Rosa26-LSL-tdTOMATO mice were removed and immediately submerged in ice-cold sucrose-based cutting solution (adjusted to pH 7.3) containing (in mM) 10 NaCl, 25 NaHCO₃, 195 Sucrose, 5 Glucose, 2.5 KCl, 1.25 NaH₂PO₄, 2 Na pyruvate, 0.5 CaCl₂, 7 MgCl₂ bubbled continuously with 95% O2 and 5% CO₂. The slices (250 μm) were cut with a Microm HM 650V vibratome (Thermo Scientific and recovered for 1 h at 34° C. and then maintained at room temperature in artificial cerebrospinal fluid (aCSF, pH 7.3) containing 126 mM NaCl, 2.5 mM KCl, 2.4 mM CaCl2, 1.2 mM NaH₂PO₄, 1.2 mM MgCl₂, 11.1 mM glucose, and 21.4 mM NaHCO₃ saturated with 95% O₂ and 5% CO₂. tdTOMATO(+) neurons were visualized using epifluorescence and IR-DIC imaging on an upright microscope equipped with a moveable stage (MP-285, Sutter Instrument). Individual neurons were manually picked up by the pipette and 20 neuron cells were combined as a sample for qPCR analysis of Ptprd and Olfr734.

In mice with unilateral knock out of Piprd using CRISPR-Cas9, six weeks after the stereotaxic injections of AAV-FLEX-saCas9+AAV-Ptprd/sgRNA-FLEX-GFP on one side and AAV-Ptprd/sgRNA-FLEX-GFP+AAV-mCherry (no Cas9) on the other side, electrophysiology recording of GFP-labeled or mCherry-labeled AgRP neurons was performed in AgPR-IRES-Cre mice. Mice maintained under ad libitum feeding or after overnight fasting were deeply anesthetized with isoflurane and were transcardially perfused, brain slices containing the ARH prepared and maintained in artificial CSF as described above. GFP or mCherry-labeled neurons in the ARH were visualized using epifluorescence and infrared-differential interference contrast (IR-DIC) imaging on an upright microscope (Eclipse FN-1, Nikon).

For electrophysiological recording, brain slices were superfused at 34° C. in oxygenated artificial CSF at a flow rate of 1.8-2 ml/min. Patch pipettes with resistances of 3-5 MΩ were filled with intracellular solution (pH 7.3) containing 128 mM K-Gluconate, 10 mM KCl, 10 mM HEPES, 0.1 mM EGTA, 2 mM MgCl₂, 0.05 mM Na-GTP and 0.05 mM Mg-ATP. Recordings were made using a MultiClamp 700B amplifier (Axon Instrument), sampled using Digidata 1440A, and analyzed offline with pClamp 10.3 software (Axon Instruments). Series resistance was monitored during the recording, and the values were generally <10 MΩ and were not compensated. Data were excluded if the series resistance increased dramatically during the experiment or without overshoot for the action potential. Currents were amplified, filtered at 1 kHz, and digitized at 20 kHz. The current clamp was engaged to measure neural firing frequency and resting membrane potential (RM) in control and Ptprd KO AgRP neurons from fed and fasted mice. Some experiments were performed in brain slices from fed mice after one-hour incubation with GFP or 34 nM asprosin prior to recording.

In another experiment, brain slices containing the ARC were prepared from AgRP-IRES-Cre/Rosa26-LSL-tdTOMATO mice (10-12 weeks age) using same method described above. tdTOMATO(+) AgRP neuron depolarization and firing rate were recorded in response to asprosin alone and asprosin preincubated with rPTPRD-LBD or a physiologically irrelevant protein (GFP). For this, rAsprosin was preincubated with either PTPRD-LBD or a physiologically irrelevant protein (GPF) at a 1:10 ratio and then kept on ice for 1 h before treating the brain. tdTOMATO(+) AgRP neurons were first exposed to a puff of rAsprosin (34 nM), followed by perfusion with a mixture of rAsprosin+PTPRD-LBD (or rAsprosin+GFP), each for 4 minutes. After a wash, AgRP neuron were treated with another puff of asprosin (34 nM).

### Adenovirus Injection

Twelve-fourteen week-old normal chow fed lean Ptprd^{-/-} and Ptprd^{+/+} (WT; wildtype) littermate male mice were injected intravenously via the tail-vein with adenovirus (Ad5) as previously described. Mice injected with Ad5-empty (3.6×10⁹ pfu/mouse) served as controls for experimental mice that received Ad5-FBN1 virus (3.6×10⁹ pfu/mouse) containing the human FBNl coding region under control of a CMV promoter. Mice injected with Ad5-empty (5×10¹⁰ pfu/mouse) served as controls for experimental mice that received Ad5-IL2-Asprosin (5×10¹⁰ pfu/mouse) containing an N-terminal his-tagged human asprosin coding region preceded by an IL2 signal peptide, under control of an EFI promoter.

Eighteen-week-old DIO (diet induced obese; C57BL/6J) male mice were intravenously injected via the tail vein with Ad5-IL2-hPTPRD-LBD or Ad5-eGFP (1×10¹¹ vp/mouse or 2×10¹² vp/mouse). Mice transduced with Ad5-eGFP served as controls for experimental mice that received Ad5-IL2-hPTPRD-LBD containing an N-terminal his-tagged ligand-binding-domain (LBD) of PTPRD coding region preceded by an IL2 signal peptide, under control of an EF1 promoter.

### Western Blot Analysis

HEK293T cell protein lysates were prepared in Pierce immunoprecipitation lysis buffer (ThermoFisher Scientific, 87787) supplemented with 1× Halt protease inhibitor cocktail (ThermoFisher Scientific, 87786). 5 or 10 μg cell lysate samples were mixed with Bolt LDS Sample Buffer (Fisher Scientific, B00008) and run on a Bolt 4-12% Bis-Tris Plus gradient gel (Thermo Fisher Scientific). Protein lysate from Snapfrozen hypothalamic tissue of Ptprd^{-/-} and WT mice was prepared using N-PER Neuronal protein extraction reagent (Thermo Fisher, 87792) supplemented with protease inhibitor and phosphatase inhibitor. 60 μg protein lysate samples were mixed with NuPAGE™ sample buffer (Fisher Scientific, NP0007) and run on a NuPAGE™ 3 to 8%, Tris-Acetate gel (Thermo Fisher Scientific). Proteins were transferred to nitrocellulose membranes using the Invitrogen Power Blotter for 7-10 minutes at room temperature. The membranes were blocked with Clear Milk Blocking Buffer or 5% BSA blocking buffer (in 1× TBST). Membranes were incubated with primary antibody at 4° C. overnight followed by incubation with HR-labelled secondary antibody for 2 hr at room temperature. HRP was detected using chemiluminiscent substrates (Thermo Fisher Scientific, 34577, 34094). Membranes blotted for p-Stat3 were stripped, washed and reblotted for stat3 detection.

Antibodies used were rabbit polyclonal anti-PTPRD (1:500; ABclonal, A15713), mouse monoclonal anti-asprosin (1:2000), mouse monoclonal anti-stat3 (1:2000; Cell signaling technology, 9139), rabbit monoclonal anti-phospho Stat3-Tyr705 (1:500; Cell signaling technology, catalog #9145); Mouse BActin (1:2000; Cell signaling; 8H10D10) mAb; HRP-conjugated anti-rabbit (1:10,000; GE Healthcare), and HRP-conjugated anti-mouse (1:10,000; KPL Scientific).

### Quantitative PCR

Ptprd expression was measured in hypothalamus, cerebellum, heart, liver, white adipose tissue, brown adipose tissue, and skeletal muscle of wild type mice. Expression of PTPRD was measured in liver of mice transduced with Ad5-GFP and Ad5-PTPRD-LBD, and in HEK293T cells transfected with scrambled or PTPRD siRNA. Aliquots of these tissues and cells were used for RNA extraction using the RNeasy kit (Qiagen) following manufacturer's instructions. First-strand cDNA was synthesized with Iscript™ cDNA synthesis kit (Bio-Rad) and subjected to qPCR analysis. For assessment of Ptprd and Olfr734 in AgRP neurons, individual neurons were manually picked up by the pipette and 20 neuron cells were combined as a sample RNA extraction and reverse transcription using the Ambion Single-Cell-to-CT Kit (Ambion, Life Technologies) according to the manufacturer's instruction. Briefly, 10 μl Single Cell Lysis solution with DNase I was added to each sample, and then the entire cell contents were used for cDNA synthesis (25°° C. for 10 min, 42° C. for 60 min, and 85° C. for 5 min).

For Ribotag pulldown of transcripts from AgRP neurons, RNA was isolated using Ribo-Tag strategy as described before. Briefly, 350 μl lysate from hypothalamic punches containing the entire ARC region of AgRP-IRES-Cre/Rosa26-LSL-RiboTag mice was incubated with 1 μl anti-HA antibody (MMS-101P, Covance) for 4 h at 4° C., and then 50 μl of protein A/G agarose beads (Santa Cruz) overnight at 4° C., followed by high salt buffer wash. The RNA from inputs (before incubation) and immunoprecipitates were extracted using Qiagen RNeasy Micro Plus kit (Qiagen) and used for RT-PCR.

Real-time qPCR was performed using iTaq™ Universal Probes Supermix (Bio-Rad) and the Bio-Rad CFX96 Real-Time system (Bio-Rad). Target gene primer sets were designed to be compatible with the Universal ProbeLibrary (Roche).

### Metabolic Caging and Manual Food Intake Assessment

Metabolic caging experiments were performed at the Cardiovascular Research Institute Mouse Metabolic and Phenotyping Core at CWRU (IACUC #2019-0029). Mice were housed with a 12-h light/dark cycle (7 am/7 pm) at 22° C. with controlled humidity. Metascreen software (V2.3.15.11) controlled system data acquisition. Respirometry (VO₂, VCO₂, H₂O vapor), activity and ad libitum food and water intake measures were collected individually using a Promethion metabolic cage system (Sable Systems, Las Vegas, NV, USA). Gas analyzers were calibrated before each run. Gas measurements were multiplexed over 8 cages and baselined to a cage-equivalent volume of room air twice per 5-minute cycle, while maintaining a 2 L/min/cage, negative pressure-derived flow rate. Acquired data was processed using Macro Interpreter (V2.34) running Macro V2.33.3-slice 1 hr. Energy expenditure was calculated using the Weir equation (Weir, 1949).

In some experiments, for manual measurement of food intake, DIO mice were acclimated to crushed high fat diet (60% calories from fat, TD.06414, Envigo Teklad) in single housing for three days. The diet was replenished, weighed, and re-weighed after 24 hours to establish food intake.

### Blood Parameters

For glucose tolerance test (GTT), mice were intraperitoneally injected with glucose solution (2 g/kg body mass) and blood glucose levels were measured at 0, 15-, 30-, 60- and 120-min post treatment. Mouse glucose was determined using a hand-held glucometer (OneTouch Ultra2, LifeScan) from a droplet of tail blood. Plasma Asprosin and Ghrelin levels were measured using mouse asprosin (Amsbio, AMS.ELK6516 and Abbexa, abx585287) and mouse/rat total Ghrelin (EMD Millipore, EZRGRT-91K) ELISA kits. Mouse plasma total cholesterol, HDL (high-density lipoprotein cholesterol), LDL (low-density lipoprotein cholesterol), triglyceride (TG), free fatty acids (FFA), glycerol, leptin, and adiponectin levels were measured by the mouse metabolism and phenotyping core at Baylor College of Medicine, Houston, Texas (MMPC at BCM & NIH fund RO1DK114356 & UM1HG006348).

### Phospho-Stat3 Assays and Luciferase Assays

### In Vivo Assessment of Phospho-Stat3

16-week-old lean wildtype mice were fasted overnight followed by 2 hours of refeeding, two weeks post transduction with ad5-hFBNl or ad5-empty (3.6×10⁹ pfu/mouse). Mice were decapitated under deep isoflorane anesthesia and hypothalamicised, snap-frozen, and later subjected to phosphor-stat3 quantification by ELISA (Abcam; ab126458), and by western blot analysis of β-actin, stat3 and phospho-stat3. Similarly, 16-week-old Ptprd^{-/-} and wildtype (WT) normal chow fed male littermates were subjected to overnight fast followed by 2 h refeeding, for assessment of hypothalamic phosphor-stat3 quantification by western blot and ELISA. To compare pSTAT3 in animals matched for adiposity, phosphorylated-Stat3 (p-Stat3) levels were measured using ELISA in hypothalamic neural lysate of overnight fasted 20-week-old diet-induced obese (DIO) male mice, 16 h post intraperitoneal treatment with anti-asprosin monoclonal antibody (mAb) or IgG control antibody (500 μg in 500 ml saline/mouse; n=6/group).

### In Vitro Phospho-Stat3 Studies

Phospho-stat3 levels were quantified by western blot and luciferase readout in HEK293T cells under conditions of PTPRD knockdown, with or without asprosin overexpression.

To evaluate the effect of PTPRD loss on Stat3 activity, HEK293T cells were co-transfected with 2 μg pTATA-TK-Luc (2 μg) and PTPRD siRNA (25 nM) or scrambled siRNA (25 nM). Cells were lysed for assessment of siRNA efficiency, and phospho-Stat3 quantification by western blot and luciferase readout, 72 hours post transfection. In another experiment, Stat3-response element driven luciferase activity was assessed in HEK293T co-transfected with pTATA-TK-Luc (1 μg/well) and serial dilution of asprosin-coding mammalian expression plasmid, with empty plasmid as control treatment (2 μg, 1 μg, 0.5 μg and 0.25 μg/well). Luciferase assay readout was performed 72 h post transfection. To ascertain the effect of PTPRD knockdown on asprosin mediated signaling, 24 hours after PTPRD or scrambled siRNA (25 nM) treatment, asprosin was overexpressed using Ad5-asprosin or Ad5-empty (100 vp/cell) in HEK293T cells. 48 hours post asprosin overexpression, media was collected for measuring secreted asprosin levels and cells were collected for western blot of β-actin, stat3 and p-Stat3. Similarly, asprosin was overexpressed using asprosin coding mammalian expression plasmid (1 μg/well) under conditions of PTPRD knockdown (with scrambled siRNA as control) and pTATA-TK-Luc (1 μg/well) transfection, and luciferase assay was done 72 h post treatment for assessment of asprosin induced changes in cellular stat3 activity under receptor knockdown conditions.

For in vitro neutralization of asprosin, HEK293T cells were transfected with empty or asprosin coding mammalian expression plasmid (1 μg/well). 48 h post transfection, cells were treated with lug recombinant eGFP or rPTPRD protein (Acro Biosystems, PTD-H52H9) for 8 hours. Cells were lysed for western blot of βactin, stat3 and p-Stat3. Similar experiment was done under conditions of pTATA-TK-Luc (lug/well) transfection, and luciferase assay was performed 8 hours post ectopic protein treatment. Additionally, effect of asprosin neutralization was tested in HEK293T cells expressing asprosin or cells exposed to asprosin conditioned media. For this, HEK293T cells were co-transfected with pTATA-TK-Luc (2 μg/well) and asprosin coding mammalian expression plasmid (1 μg/well) for 48 h, followed by ectopic treatment of anti-asprosin mAb or IgG control. Stat3-response element driven luciferase activity was measured 18 h post antibody treatment. Additionally, above experiment was repeated using asprosin-conditioned media. Conditioned media was collected from HEK293T cells transfected with asprosin expressing mammalian plasmid, and incubated with IgG control or anti-asprosin mAb (500 ng/ml) for 2 h. Stat3-response element driven luciferase activity was measured in pTATA-TK-Luc (2 μg/well) transfected cells, 18 h post treatment with asprosin conditioned media+IgG and asprosin conditioned media+anti-asprosin mAb.

### Mouse Body Composition, Magnetic Resonance Imaging

Mouse MRI imaging experiments were performed at the Case Center for Imaging Research. Each animal was anesthetized in isoflurane and placed in the prone position at isocenter in a Bruker Biospec 7T MRI scanner. A 72-mm inner diameter volume coil was used to acquire the images. Following initial localizer scans, a fat-water technique was used to obtain three separate high resolution coronal image sets with multiple echo shifts in order to generate separate fat and water images using the Relaxation-Compensated Fat Fraction (RCFF) MRI as described previously. These quantitative fat fraction maps were then used to automatically segment out peritoneal and subcutaneous adipose tissue in each imaging slice. The total volume of the peritoneal and subcutaneous adipose tissue was then calculated by summing over all imaging slices.

### Asprosin and PTPRD Ligand Binding Domain ELISA Detection Procedures

Media collected from HEK293T cells transduced with Ad5-asprosin or Ad5-empty was subjected to asprosin Elisa, as previously described. For validation of asprosin sequestration with PTPRD-ligand binding domain, 5 nM solution of recombinant human asprosin preincubated with recombinant PTPRD or GFP protein was subjected to asprosin ELISA as previously described. For detection of human asprosin in plasma of mice treated with human asprosin and FBNl expressing Ad5 vectors, plasma samples were first processed for IgG and albumin removal using Proteome purify2 columns (R & D systems, Catalog #IDR002) and concentrated using vivaspin 500 (VS0131) PES filters before running the ELISA.

Briefly, a sandwich ELISA custom built using mouse monoclonal anti-asprosin antibody against human asprosin amino acids 106-134 (human profibrillin amino acids 2838-2865) as the capture antibody, and a rabbit anti-asprosin monoclonal antibody as the detection antibody was used for detection of human asprosin. An anti-rabbit secondary antibody linked to HRP was used to generate a signal, and mammalian-cell produced recombinant human asprosin was used to generate a standard curve.

For detection of human PTPRD ligand binding domain (LBD) in plasma of mice treated with PTPRD-LBD expressing adenoviruses, a sandwich ELISA was custom built using rabbit anti-PTPRD (100 ng/well; ABclonal; A15713) as the capture antibody, and mouse anti-his (100 ng/well; Genscript; A00186) as the detection antibody. An anti-mouse secondary antibody linked to HRP was used to generate a signal, and recombinant PTPRD (Acro Biosystems, PTD-H52H9) was used to generate a standard curve.

### Quantification and Statistical Analyses

All results are presented as mean±standard error of the mean (s.e.m.). Statistical significance was tested using unpaired two tailed t-tests or Analysis of Variance (one-way and two-way ANOVA, when appropriate) followed by the Bonferroni multiple test corrections post-hoc analysis using GraphPad Prism 6 & 7. For metabolic caging experiments, data was additionally analyzed using analysis of covariance (ANCOVA) to account for covariates on R software (4.0.3). Alpha (α) for statistical significance was set at 0.05.

### Results

### Identification of an Asprosin-Interacting Receptor in the Mouse Brain

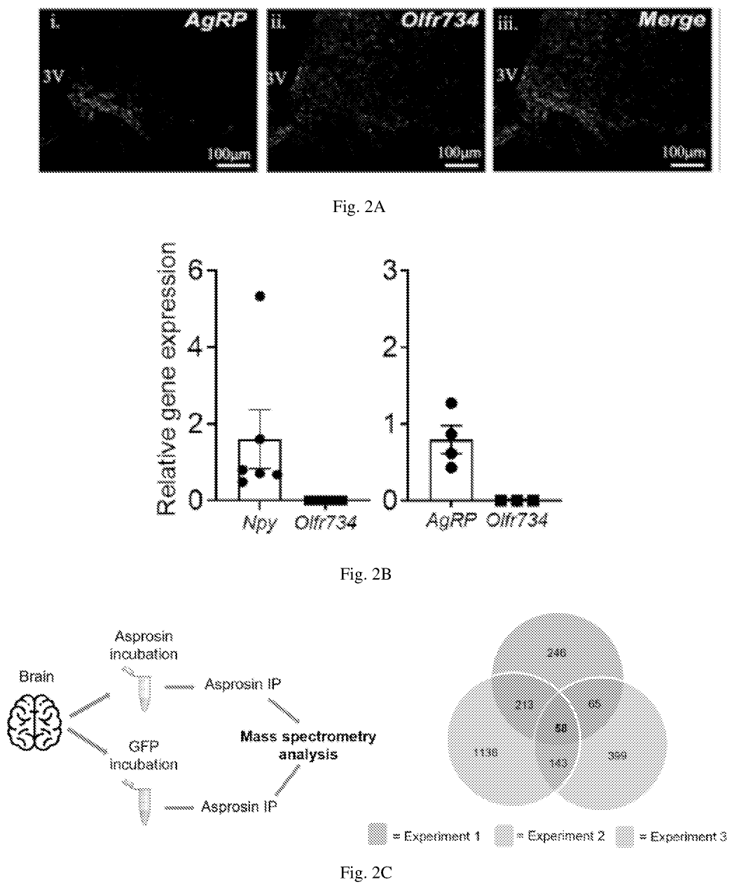

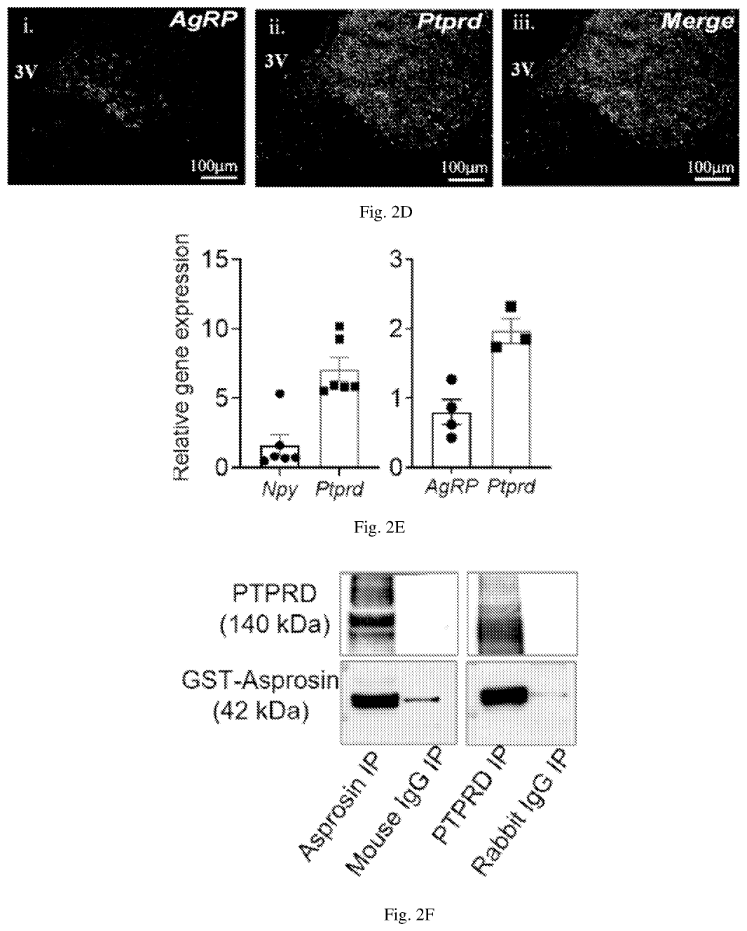

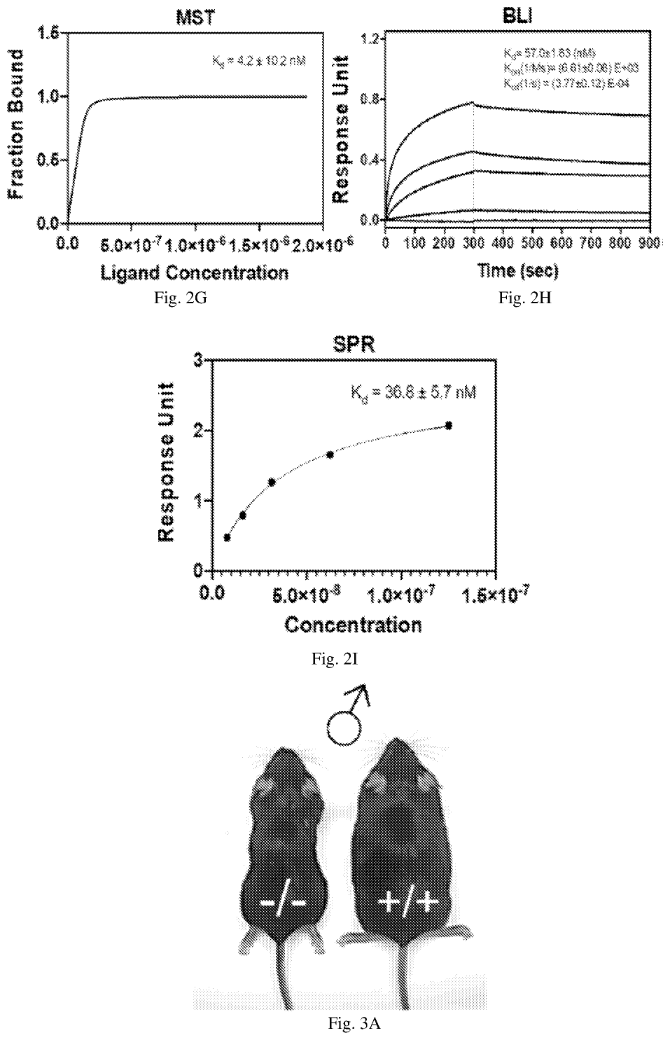

We confirmed the reported absence of the Olfr734 mRNA in mouse AgRP neurons via three independent methodologies. To that effect, we performed fluorescence RNAscope hybridization labeling for AgRP and Olfr734 transcripts in the hypothalamus of wild type mice and found no evidence of coexpression of Olfr734 and AgRP transcripts (FIG. 2A). Further, Olfr734 mRNA was undetectable by qPCR in visually enriched tdTOMATO expressing AgRP neurons (FIG. 2B), and in ribo-tag pulled-down active transcripts from AgRP-Cre mice (FIG. 2B). Given our inability to detect Olfr734 expression in AgRP neurons using multiple techniques, and the lack of concordance between Olfr734 loss and genetic asprosin deficiency or pharmacologic asprosin inhibition when it comes to body weight and ad libitum appetite deficits, we ruled out Olfr734 as a potential orexigenic asprosin-receptor.

To identify the orexigenic receptor for asprosin, we incubated recombinant asprosin or GFP with mouse brain homogenate followed by immunoprecipitation with a high-affinity anti-asprosin mAb and subjected the immunoprecipitates to mass spectrometry screening for potential asprosin-interacting proteins (FIG. 2C). Of the 58 proteins that were identified as potential asprosin-interactors in three separate experiments, we only found one membrane-bound receptor—Ptprd—was found to be strongly expressed in AgRP neurons. Olfr734 was not detected as an asprosin-interacting protein in any of the three experiments. Therefore, we focused on the plausibility of Ptprd to function as an asprosin-receptor and modulate asprosin-mediated AgRP neuron activation and orexigenic and body weight modulating functions.

Ptprd (protein tyrosine phosphatase receptor type δ), encoded by the Ptprd gene, is a single-pass membrane receptor belonging to the leukocyte common antigen-related protein (LAR)/type IIa class of PTPs (protein tyrosine phosphatases). Similar to other important appetite modulating receptors such as LEPR, GLP-IR and MC-4R, Ptprd is extensively expressed in the brain. The association of PTPRD variants with several neuronal disorders including addiction, restless leg syndrome, neurofibrillary pathology in Alzheimer's disease, obsessive-compulsive disorder and cognitive impairment has stimulated an interest in its neurobiology and genomics. While the precise neural function of Ptprd remains unknown, its intracellular phosphatase domain is reported to function as a regulator of phosphorylation-based signaling in the brain and peripheral tissues. Its extracellular domain has been implicated as a synaptic specifier and neural cell adhesion molecule; however, with no ligands identified, Ptprd is characterized as an orphan receptor.

To establish the relevance of Ptprd in asprosin's orexigenic signaling, we first verified the expression of Ptprd in AgRP neurons employing three distinct methodologies. Fluorescence RNAscope hybridization analysis indicated a strong overlap between AgRP and Piprd transcripts in the hypothalamus of wild type mice (FIG. 2D). Further, high expression of Ptprd was seen in visually enriched tdTOMATO expressing AgRP neurons, and in ribo-tag pulled down active transcripts from AgRP-Cre mice (FIG. 2E). These results confirmed that Ptprd is strongly expressed in AgRP neurons.

Asprosin Interacts with the Ptprd Extracellular Domain with Nanomolar Affinity

Reciprocal co-immunoprecipitation assays biochemically confirmed asprosin binding with the PTPRD extracellular domain (FIG. 2F).

Next, three different techniques, namely Microscale thermophoresis (MST), Biolayer interferometry (BLI) and Surface plasmon resonance (SPR) were employed to assess the affinity (K_{D}) of asprosin for the PTPRD extracellular domain (FIGS. 2G-I). The three assays found single-to double-digit nanomolar affinity between the two proteins (MST: 4.2±10.2 nM; BLI: 57±1.83 nM; SPR: 36.8±5.7 nM; FIGS. 2G-I), well within the range typically displayed by hormone-receptor interactions.

Ptprd Ablation Phenocopies the Body Weight and Feeding Deficits Associated with Genetic Asprosin Deficiency and Pharmacologic Asprosin Inhibition

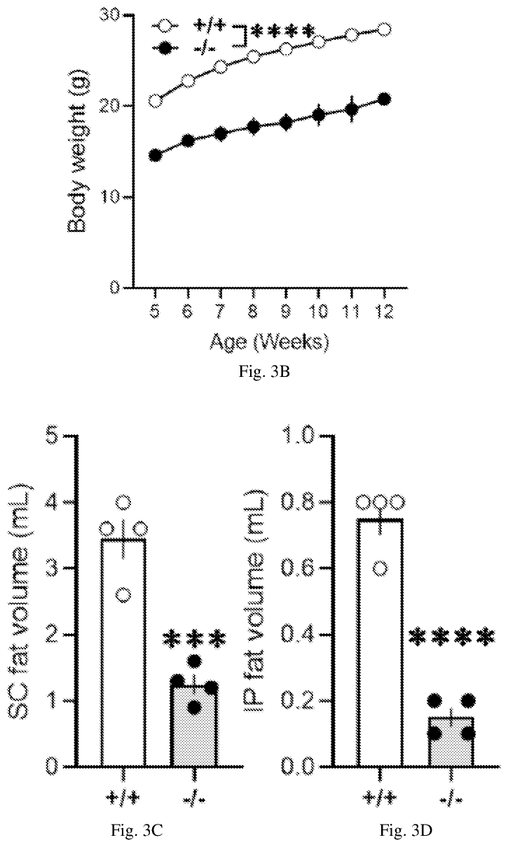

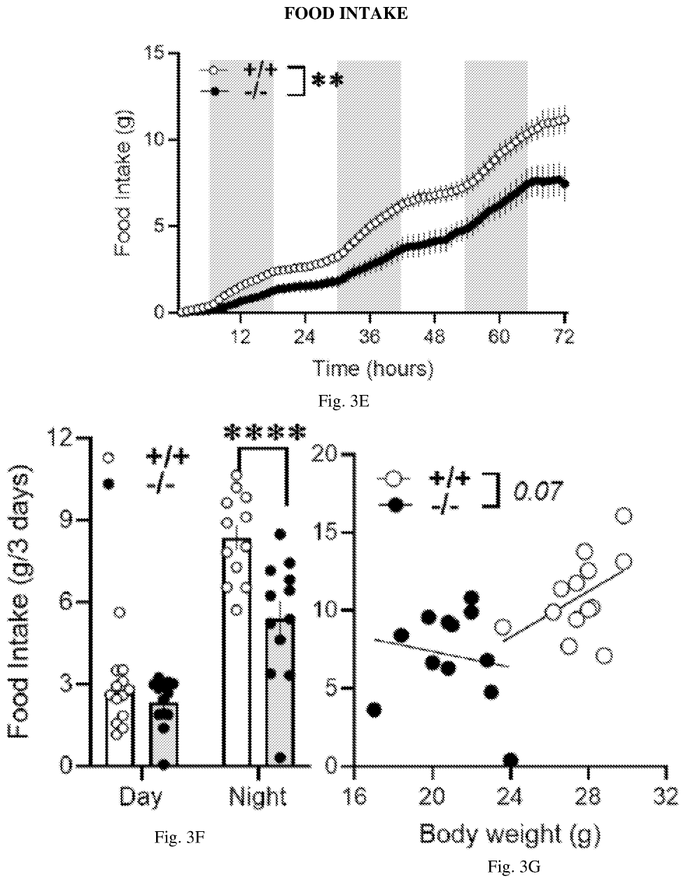

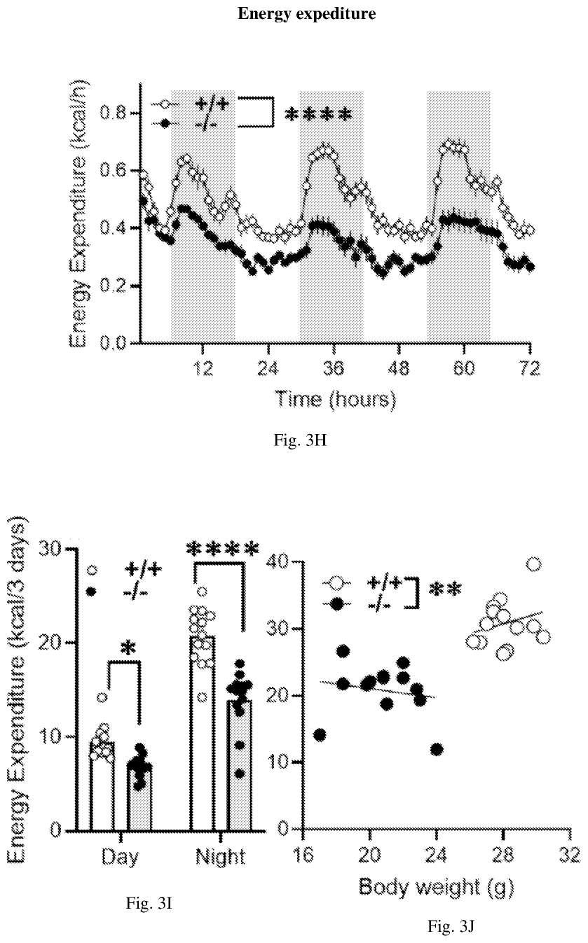

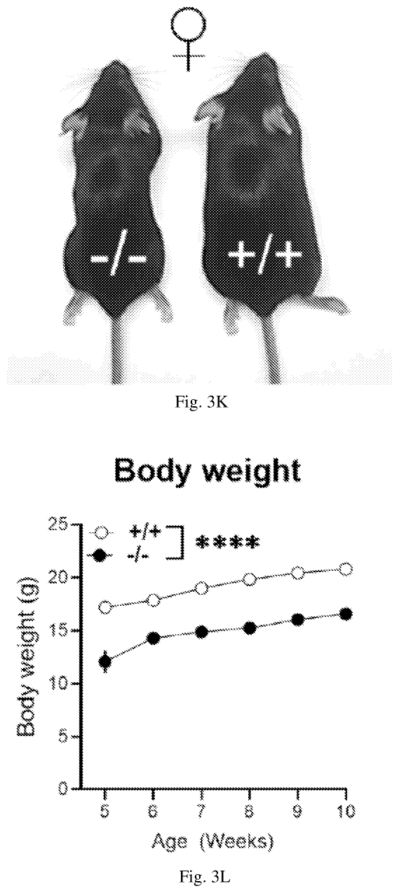

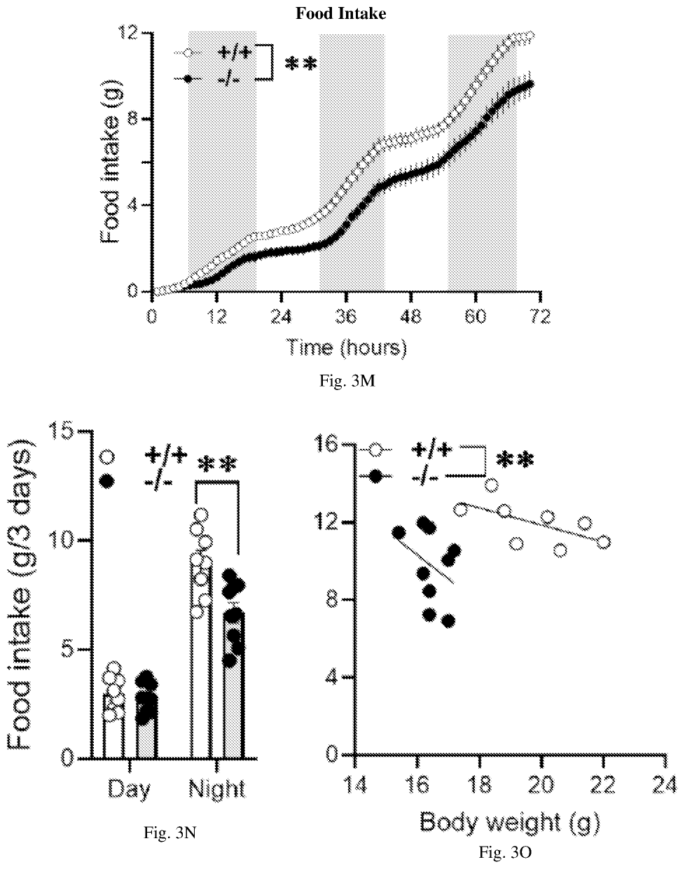

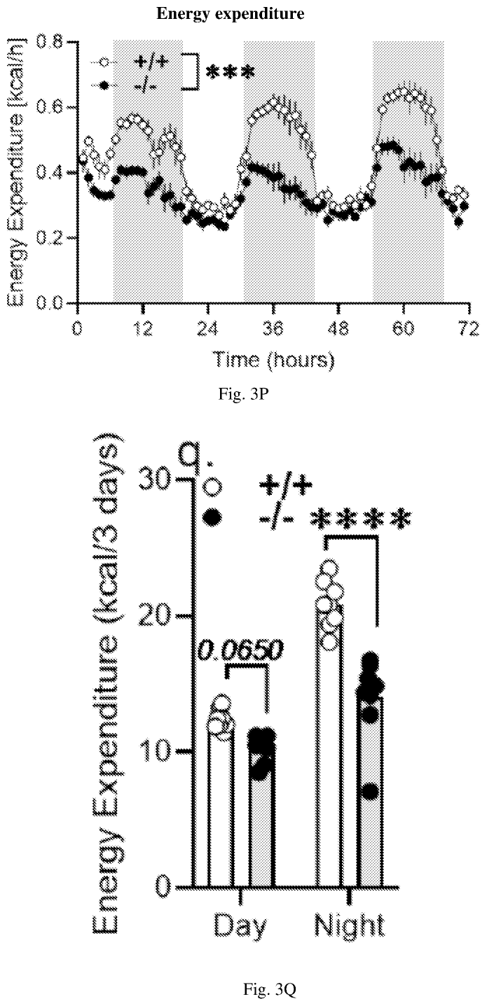

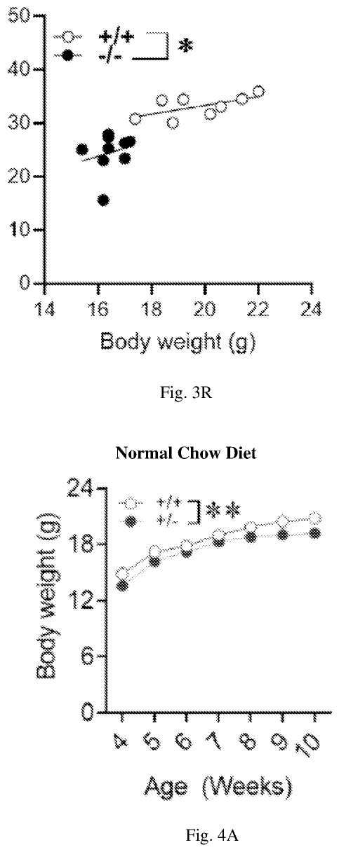

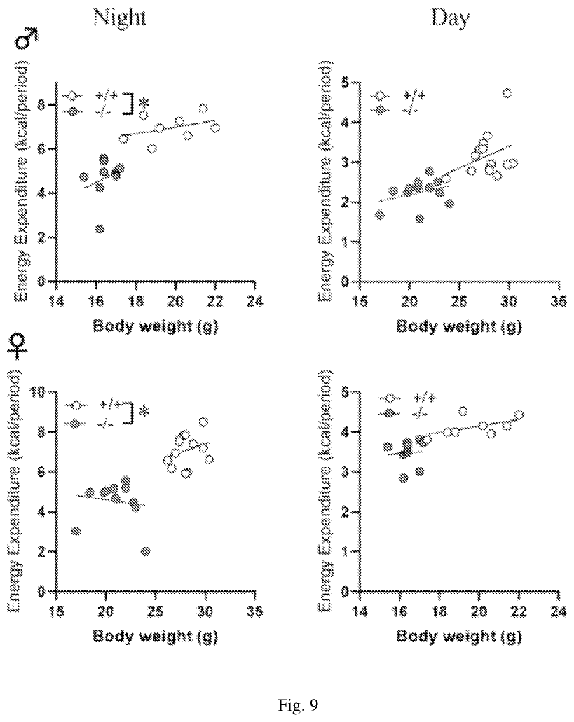

Ptprd^{-/-} mice of both sexes were found to be markedly lean and hypophagic, when compared to their WT littermates (FIGS. 3A-F, K-N). The hypophagia was accompanied by a likely compensatory reduction in energy expenditure (FIGS. 3H, I, P, Q; FIG. 9). Energy expenditure and food intake were both found to be significantly lower in Ptprd^{-/-} mice compared with WT upon analysis of covariance (ANCOVA; FIGS. 3G, J, O, R). ANCOVA statistically adjusts for the influence of total body mass disparity from group comparisons of energy expenditure. Thus, the reduced food intake and energy expenditure observed in Ptprd^{-/-} mice is independent of lower body weight. Further, the lower energy expenditure displayed by Ptprd^{-/-} mice during the active (dark) phase normalized during the inactive (light) phase (FIGS. 3I, Q; 9), suggesting that loss of Ptprd has little impact on core basal metabolic rate, and that the reduction in active (dark) phase energy expenditure is likely a compensatory mechanism to withstand the reduced food intake.

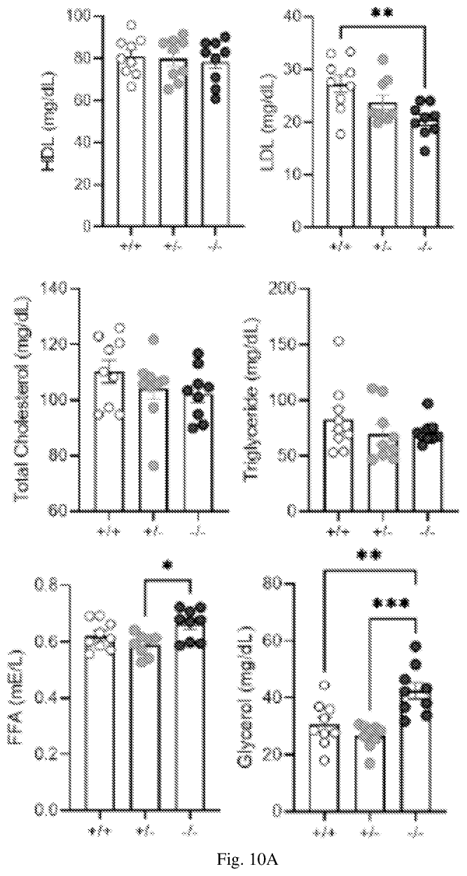

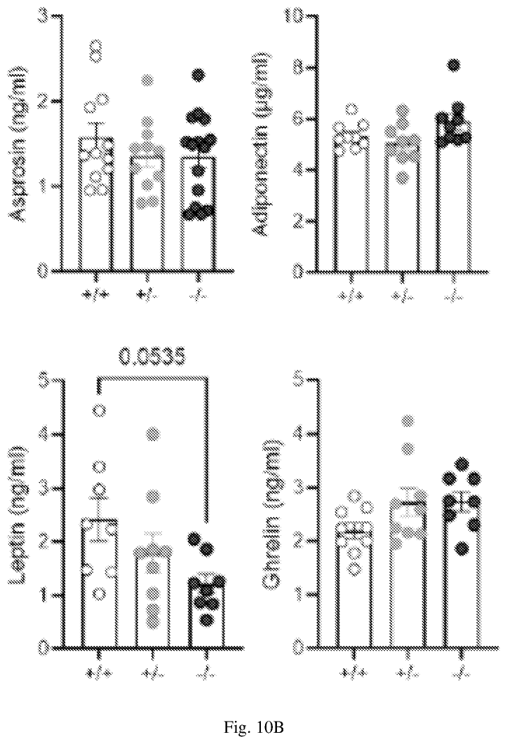

Significantly lower levels of the satiety adipokine, leptin (FIG. 10) was consistent with markedly lower subcutaneous and intraperitoneal adipose depots in Ptprd^{-/-} mice (FIGS. 3C-D). Therefore, on a comprehensive assessment of energy balance, Ptprd^{-/-} mice phenocopy Fbnl^{NPS/+} mice, a model of genetic asprosin deficiency, and mice treated with anti-asprosin mAbs, a model of pharmacologic asprosin inhibition.

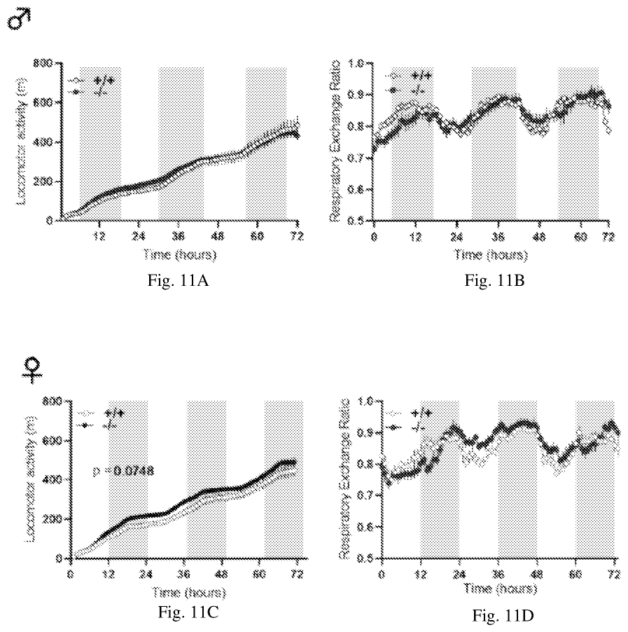

Further, increased plasma levels of free fatty acids and glycerol implicate increased mobilization of lipids to meet energy demands in hypophagic Ptprd^{-/-} mice (FIG. 10). Circulating asprosin levels were not statistically different between the Ptprd^{-/-} and WT littermates (FIG. 10). Ptprd^{-/-} mice did not display lethargy or signs of sickness in general, with no deficits seen in general locomotor activity (FIG. 11). No difference in nutrient preference via respiratory exchange ratio analysis was detected (FIG. 11) between Ptprd^{-/-} and WT littermates.

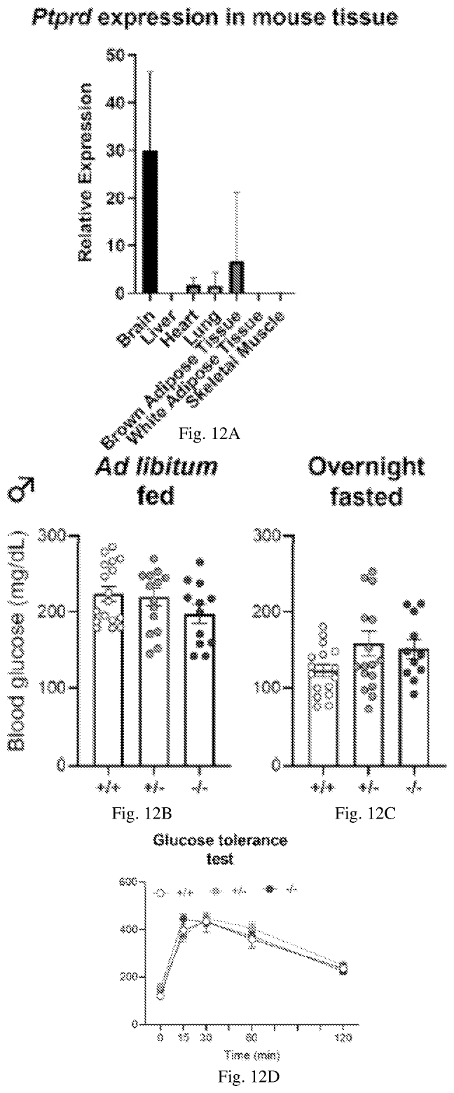

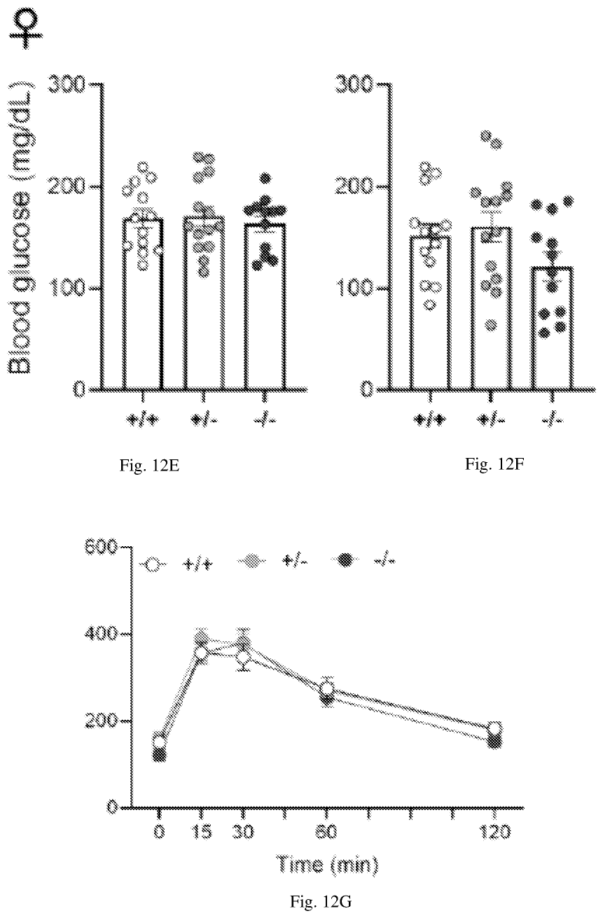

Ptprd mRNA was found to be undetectable in hepatic tissue (FIGS. 12A) from WT mice. Therefore, Ptprd seemed unlikely to modulate the peripheral effect of asprosin on hepatic glucose release. Nonetheless, plasma glucose levels were monitored under fed and fasted conditions, along with a glucose tolerance test in both male and female mice. Unlike Olfr734^{-/-} mice, Ptprd^{-/-} mice maintained euglycemia (FIGS. 12B-C, E-F) and glucose tolerance (FIGS. 12D, G) similar to age- and sex-matched WT littermates. Together these results suggested that Ptprd potentially mediates the orexigenic but not the glucogenic effects of asprosin.

### Whole Body Loss of a Single Allele and AgRP Neuron-Specific Complete Loss of Ptprd is Protective Against Diet Induced Obesity in Females

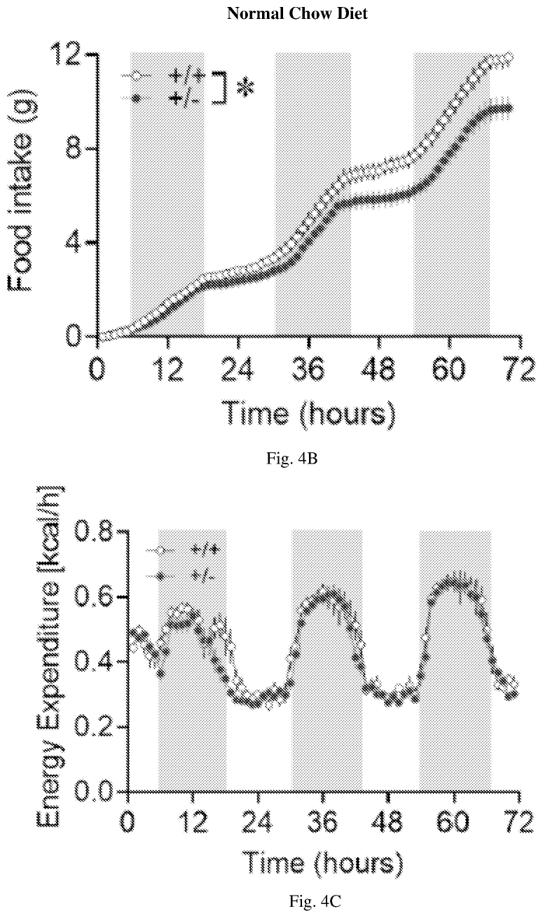

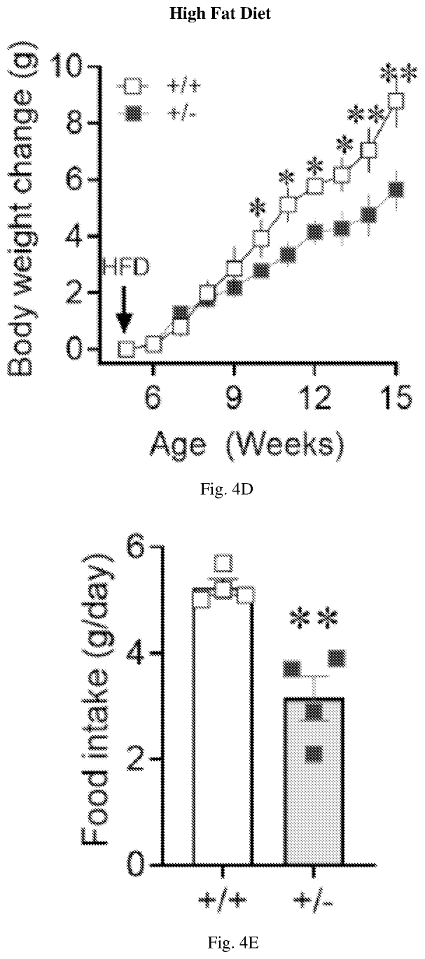

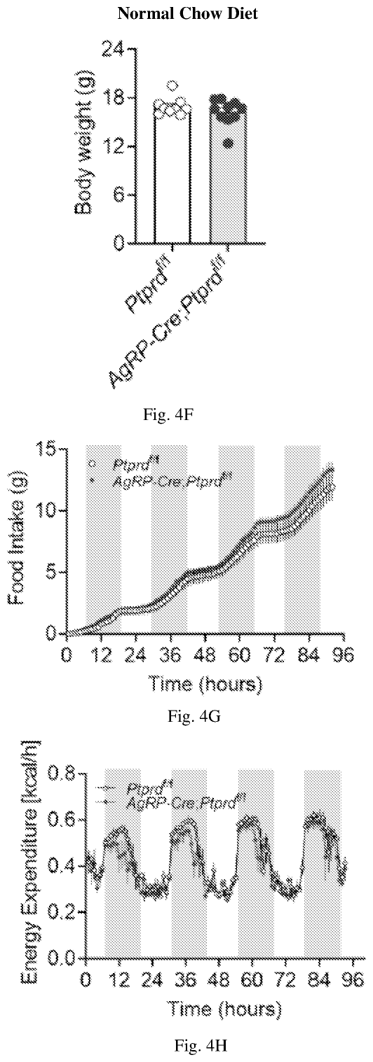

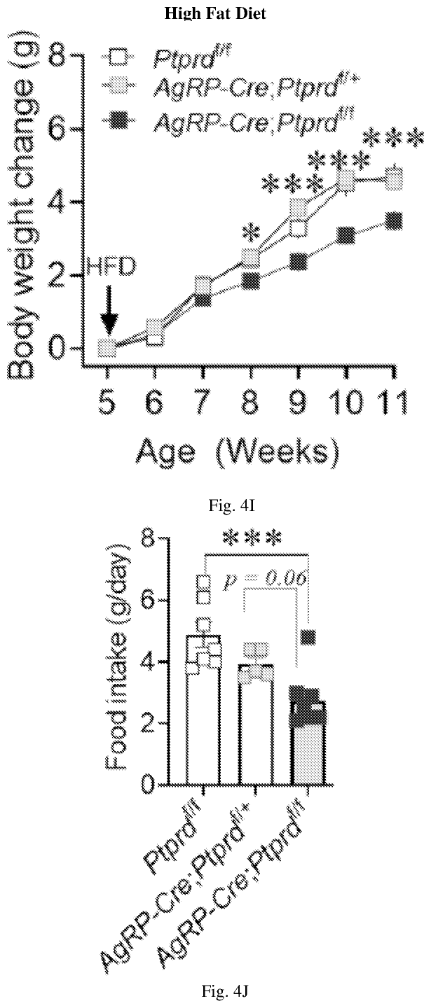

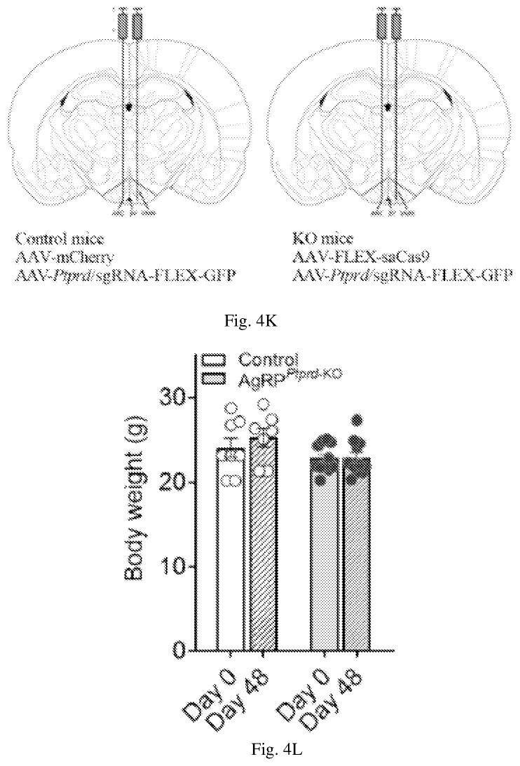

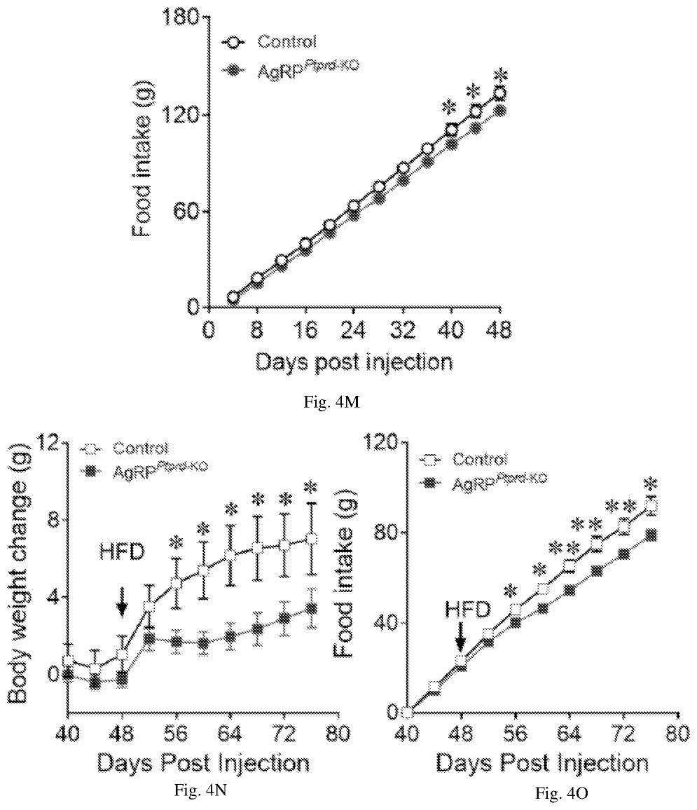

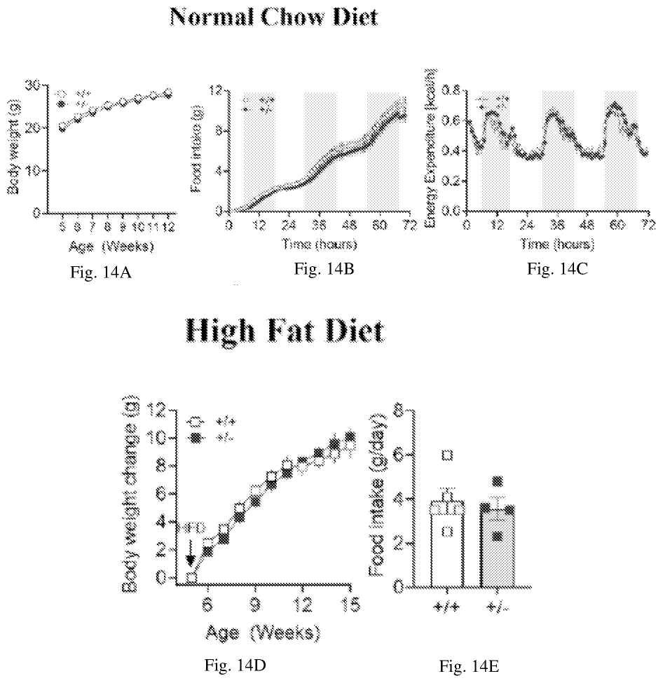

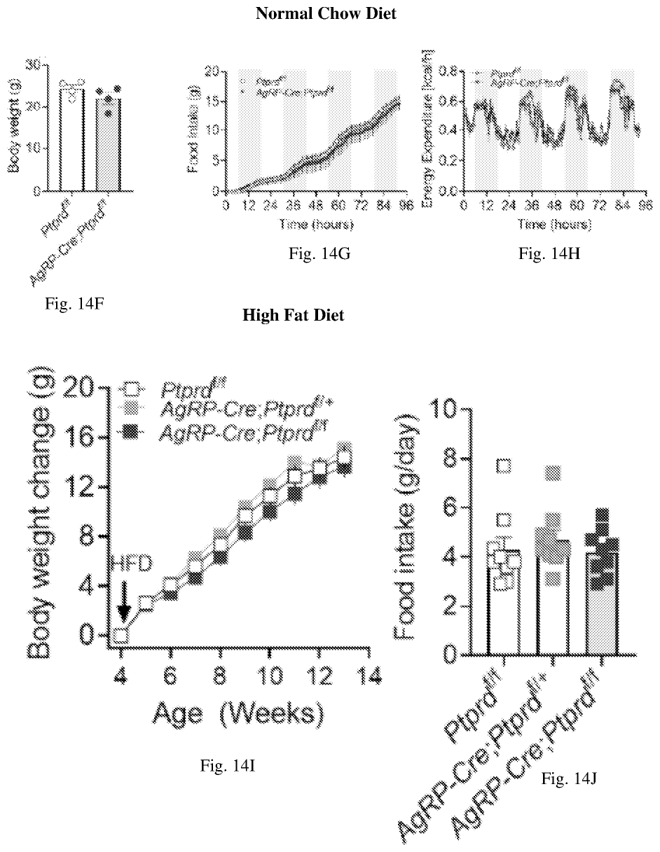

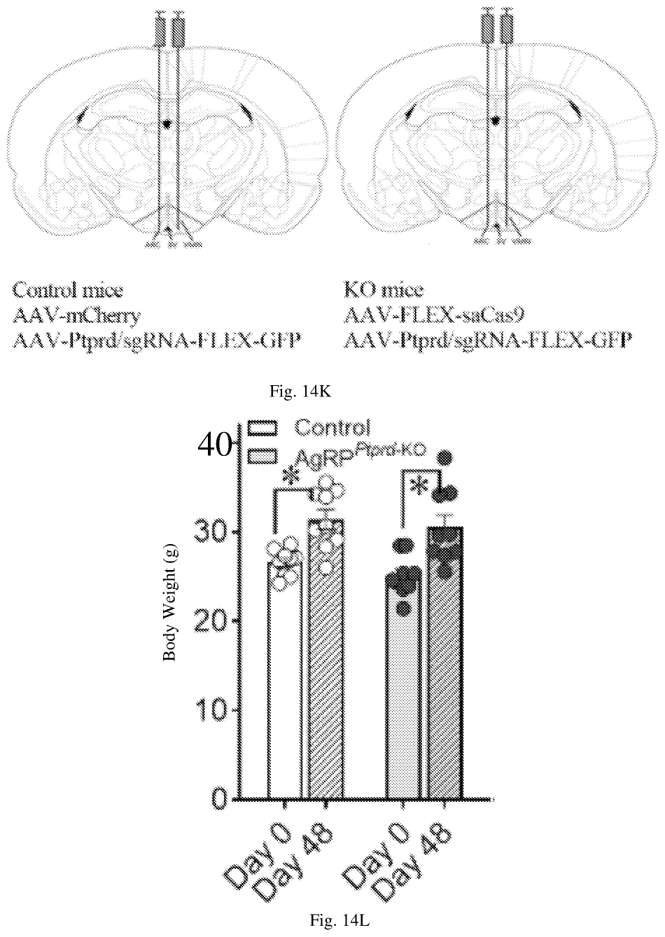

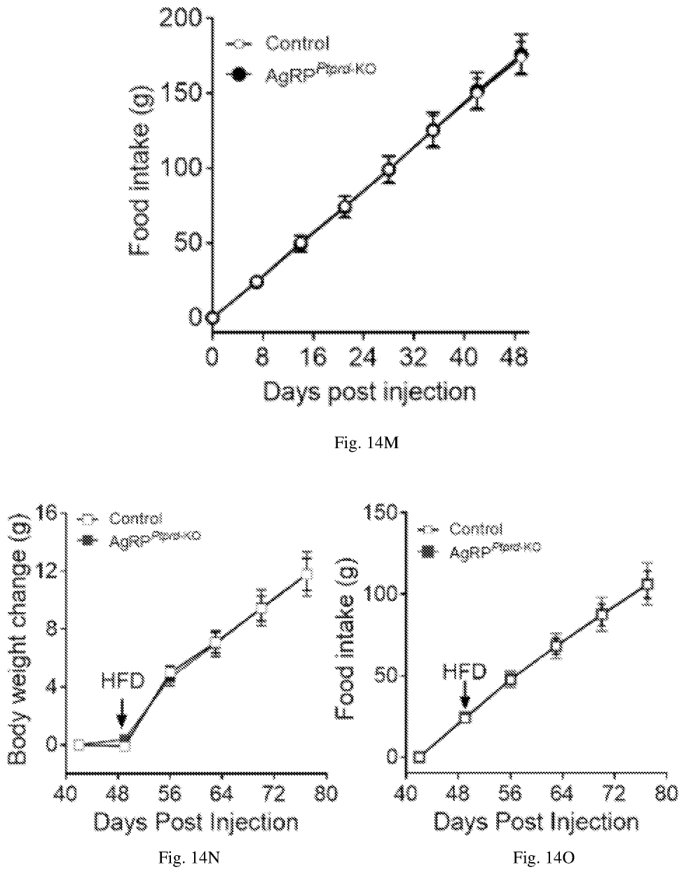

To test whether global loss of a single Ptprd allele is sufficient to perturb metabolism in mice, we tracked the body weight of Ptprd^{+/+} and Ptprd^{+/-} weanlings to adulthood on a normal chow (NC) diet. 10-week-old Ptprd^{+/-} females weighed slightly less than their WT counterparts (FIG. 4A). This marginal reduction in body weight was accompanied by a statistically significant reduction in food intake (FIG. 4B). Energy expenditure was unaltered in Ptprd^{+/-} females on NC (FIG. 4C). However, these metabolic differences between Ptprd^{+/-} and Ptprd^{+/+} females were exacerbated upon placing mice on a high fat diet (HFD). Ptprd^{+/-} females consumed nearly 40% less food and weighed nearly 4 g less than WT littermates after 10 weeks of HFD (FIGS. 4D-E). Thus, loss of a single Ptprd allele protected female mice from HFD-induced obesity. Interestingly, the metabolic consequences of loss of one allele of Ptprd were sexually dimorphic, with no differences seen in body weight, food intake or energy expenditure of Ptprd^{+/-} and Ptprd^{+/+} males on NC (FIGS. 14A-C) or HFD (FIGS. 14D, E).

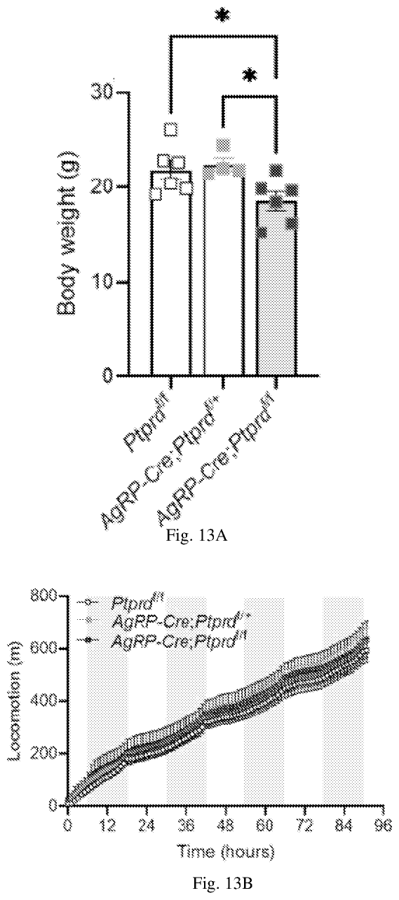

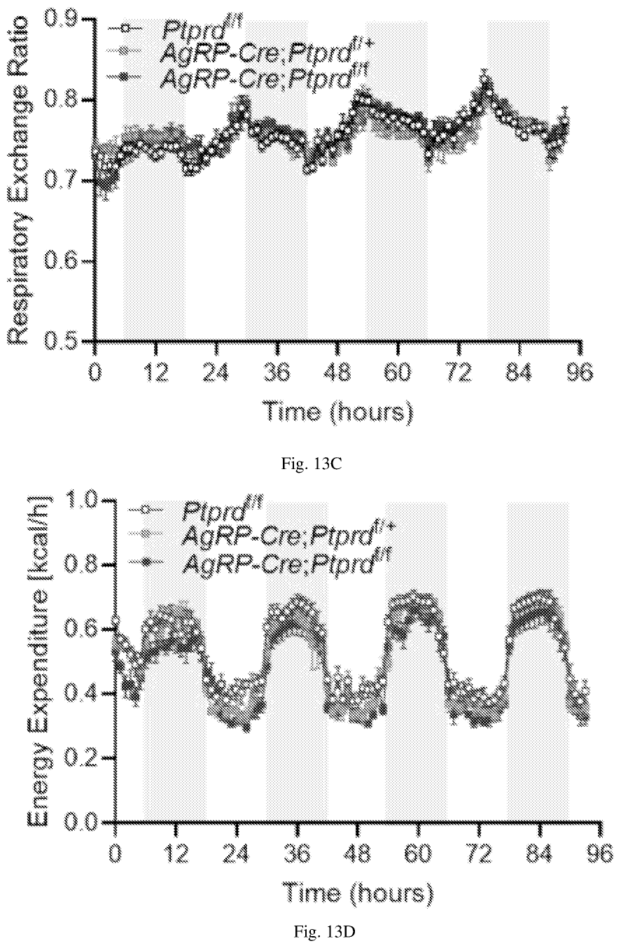

To understand the spatial relevance of Ptprd in regulation of food intake, conditional knockout of Ptprd in AgRP neurons was accomplished by crossing Ptprd^{flox/flox} mice with AgRP-Cre mice. On NC diet, AgRP-Cre⁺; Ptprd^{f/f} females were found to be metabolically similar to Piprd^{f/f} control mice, with no difference in body weight, food intake or energy expenditure (FIGS. 4F-J). This is in contrast to the marked reductions in body weight, appetite and energy expenditure seen with whole body loss of Ptprd and suggests that AgRP neuron-specific Ptprd loss on a NC diet can be compensated for by cell types with intact asprosin/Ptprd signaling. However, on a high fat diet, AgRP neuron-specific ablation of Ptprd protected female mice from diet induced obesity with AgRP-Cre⁺; Ptprd^{f/f} mice weighing significantly less than the control Ptprd^{f/f} and AgRP-Cre⁺; Piprd^{f/+} mice (FIG. 4I; FIG. 13A) after 6 weeks of high fat diet. Protection from HFD-induced obesity in AgRP-Cre⁺; Ptprd^{f/f} mice was accompanied by a significant reduction in food intake (FIG. 4J), with no changes seen in energy expenditure, activity, and respiratory exchange ratio (FIGS. 13B-D). Interestingly, male mice with AgRP neuron-specific loss of Ptprd gained weight and consumed food in quantities similar to the control Ptprd^{f/f} and AgRP-Cre⁺Ptprd^{f/+} mice (FIGS. 14I-J), again displaying a sexually dimorphic phenotype in contrast with whole body Ptprd^{-/-} mice where both sexes are equally affected.

As a third strategy, to avoid any developmental impacts of Ptprd loss, AgRP neuron-specific Ptprd ablation was accomplished in adult mice (FIGS. 4K-O). For this, Agrp-Cre mice received a bilateral stereotaxic injection of two Adeno-Associated Virus, serotype 8 (AAV) vectors in the arcuate nuclei (Arc) of the hypothalamus. The first AAV expressed a Ptprd-specific small guide RNA (sgRNA), while the second AAV expressed a cre-inducible cas9, leading to in-dels in the Ptprd gene only in AgRP neurons. Control mice received the same sgRNA as experimental mice, however the cre-inducible cas9 was replaced with mCherry. Mice were monitored for food intake and body weight changes for 80 days post stereotaxic injection. On a NC diet, day 40-48 post treatment, AgRP^{Ptprd-KO} females showed a mild reduction in food intake which was not accompanied by a body weight reduction (FIG. 4L, M). Once again, sexually dimorphic protection from HFD-induced obesity was seen with AgRP neuron-specific loss of Ptprd. Over a 40-day period on HFD, AgRP^{Ptprd-KO} female mice consumed 14g less food and gained half-as-much weight compared with control female mice (FIGS. 4N, O). AgRP^{Ptprd-KO} males consumed as much food and gained as much weight as control mice on NC and HFD (FIGS. 14L-O). These results contrast with whole body Ptprd^{-/-} mice where both sexes are equally affected, showing that sexual dimorphism only appears with less-than-total loss of Ptprd. Further, mice with AgRP neuron-specific loss of Ptprd are relatively unaffected at baseline and need to be put on a HFD for the feeding and body weight reductions to manifest. This indicates that the effects of Ptprd loss on food intake and body weight are not confounded by general sickness, learning/memory deficits or strength deficits as those would also be evident at baseline.

### Deletion of Ptprd Renders AgRP Neurons Unresponsive to Asprosin

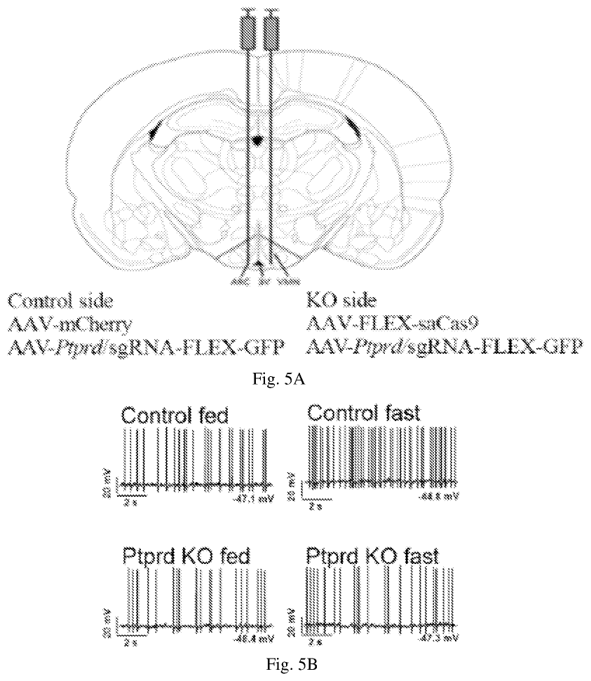

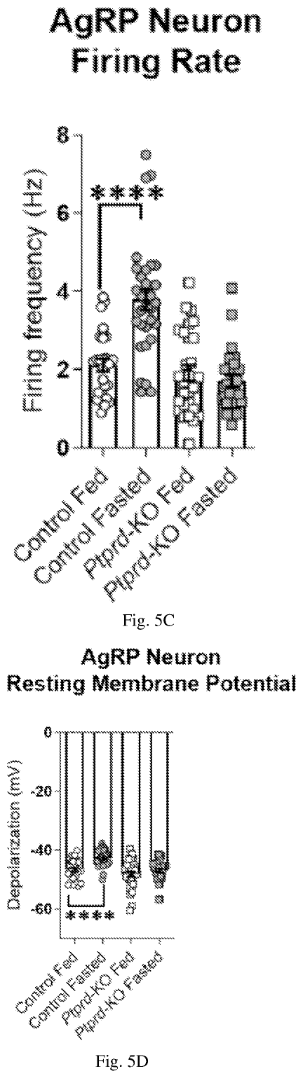

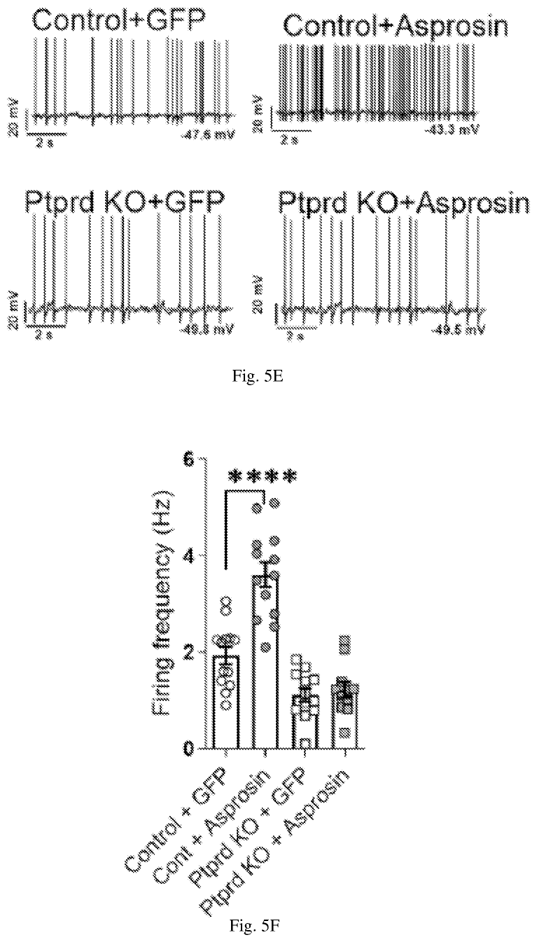

We accomplished unilateral knockout of Ptprd in AgRP neurons in adult mice to determine the impact of Ptprd loss on asprosin-mediated AgRP neuron activation (FIG. 5). For this, Agrp-Cre mice received a stereotaxic injection of two AAV vectors in the arcuate nucleus of the hypothalamus on the experimental side. The first AAV expressed a Ptprd-specific small guide RNA (sgRNA), while the second AAV expressed a cre-inducible cas9, leading to in-dels in the Ptprd gene only in AgRP neurons. On the control side, mice received the same sgRNA as the experimental side, however the AgRP-cre inducible cas9 was replaced with mCherry.

40 days after stereotaxic injections, an endogenous ‘high-asprosin’ condition was induced in mice with overnight fasting. Increased activity of AgRP neurons via an increase in firing rate and resting membrane potential upon fasting was noted in neurons from the control side. The fasting-induced increase in AgRP neuron activity was abolished in AgRP^{Ptprd-KO} neurons from the experimental side (FIGS. 5B-D). Similarly, increase in AgRP neuron activity in response to recombinant asprosin displayed by neurons from the control side was completely abolished in AgRP^{Ptprd-KO} neurons from the experimental side (FIGS. 5E-G). These results suggest absolute Ptprd necessity for asprosin-mediated AgRP neuron activation.

Ptprd^{-/-} Mice are Unresponsive to the Orexigenic Effects of Asprosin while Responding Normally to its Glucogenic Effects

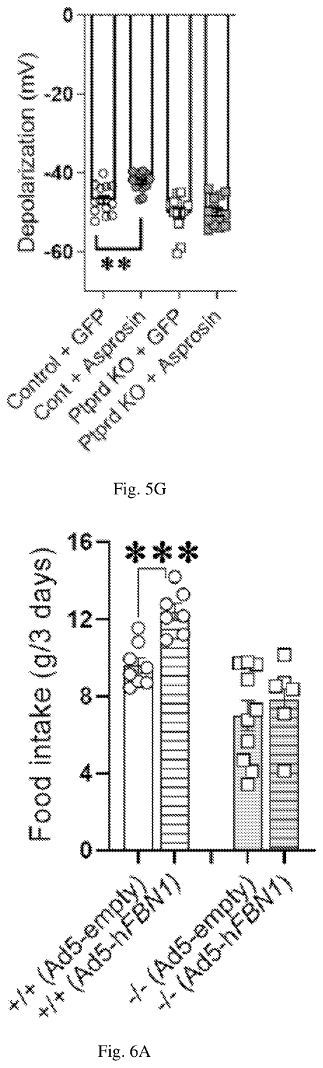

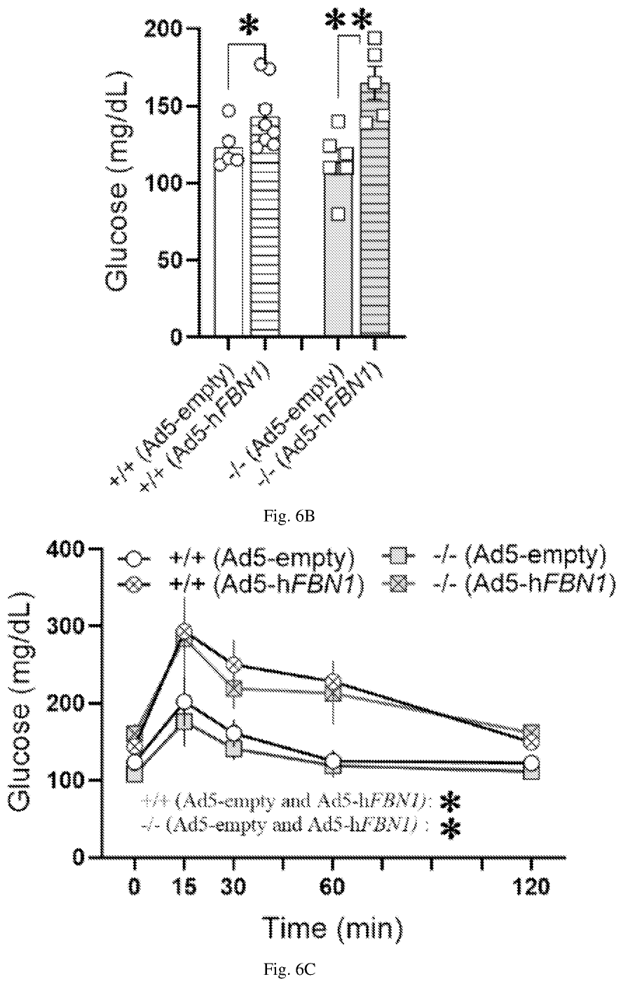

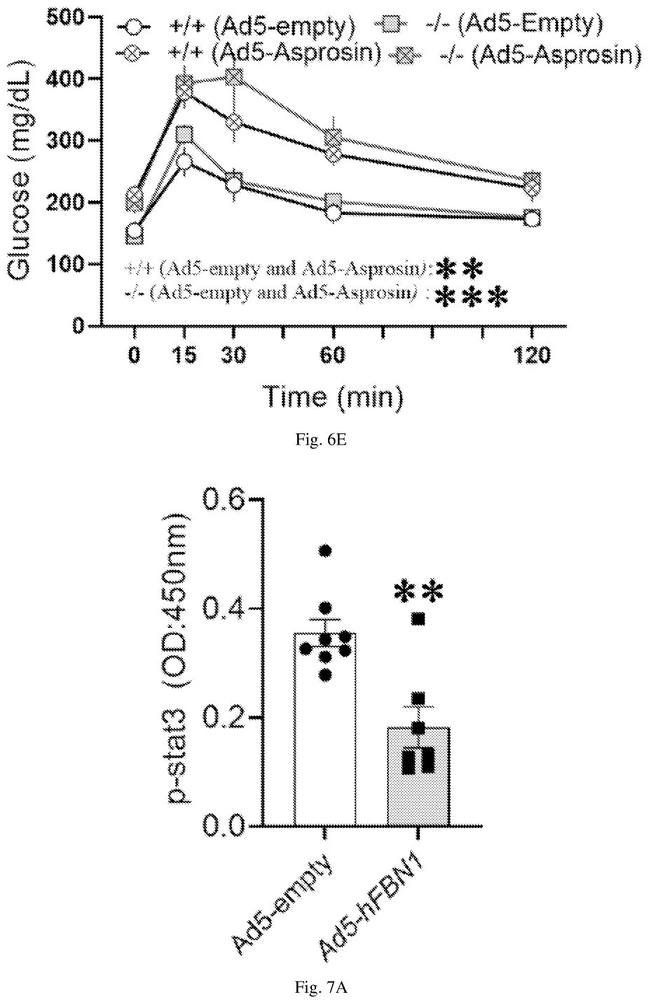

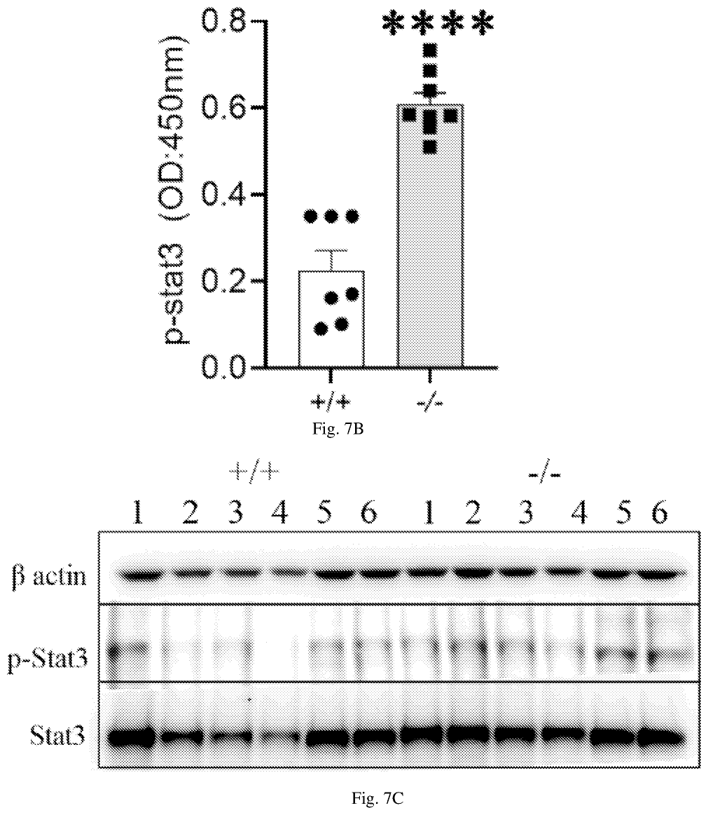

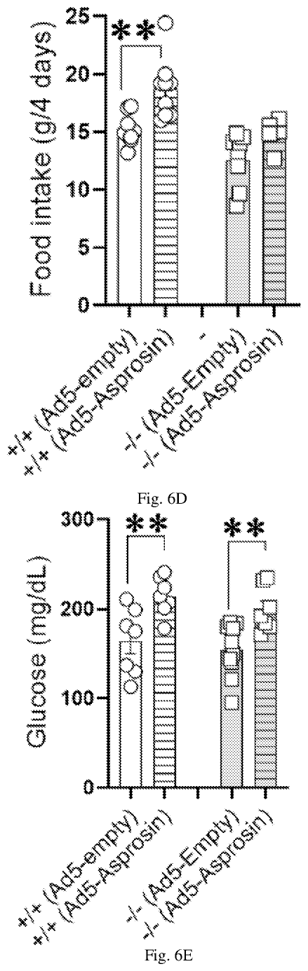

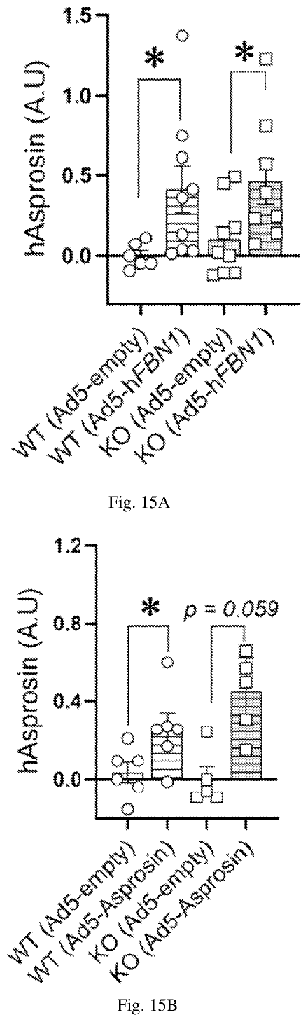

To produce sustained plasma asprosin elevation in mice without relying on recombinant preparations of variable activity, we used an adenovirus-mediated gain-of-function (GOF) strategy that we have successfully used in the past (FIG. 15). We tail-vein injected WT and Ptprd^{-/-} mice with adenoviruses (Ad5) encoding human FBNl (with native signal peptide) or human cleaved asprosin (with an IL2 signal peptide to promote secretion), and then tested whether asprosin-GOF-induced metabolic phenotypes were observed in the setting of complete Ptprd loss (FIG. 6). Like our previous reports, WT mice transduced with Ad5-FBNl or Ad5-Asprosin exhibited hyperphagia, higher baseline blood-glucose, and lower glucose tolerance when compared with Ad5-empty control mice. Interestingly, asprosin-GOF had no impact at all on appetite in Ptprd^{-/-} mice compared with the hyperphagia seen in WT mice, while the impact on baseline blood-glucose and glucose tolerance was essentially identical between WT and Ptprd^{-/-} mice. Thus, crucially, this set of experiments confirmed the absolute necessity of Ptprd for asprosin-mediated appetite stimulation, and its complete dispensableness for asprosin-mediated glucogenesis.

### Asprosin Activates Ptprd in a Cell Autonomous Manner

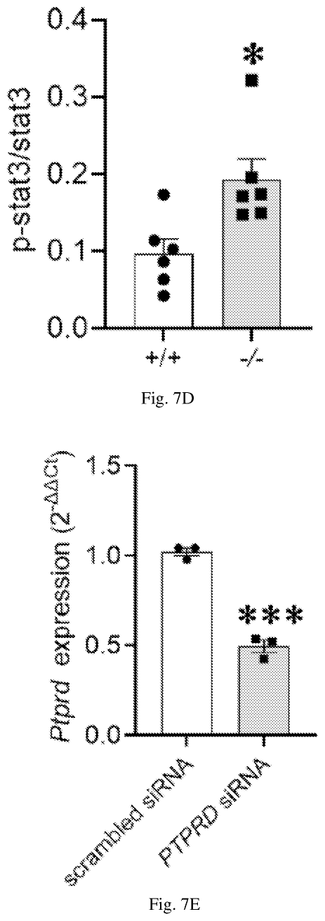

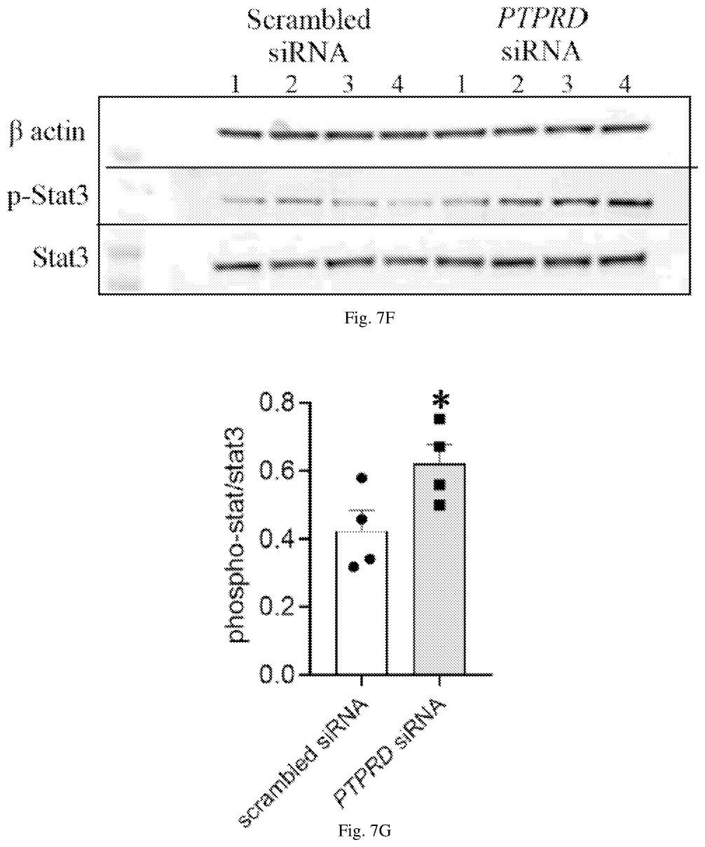

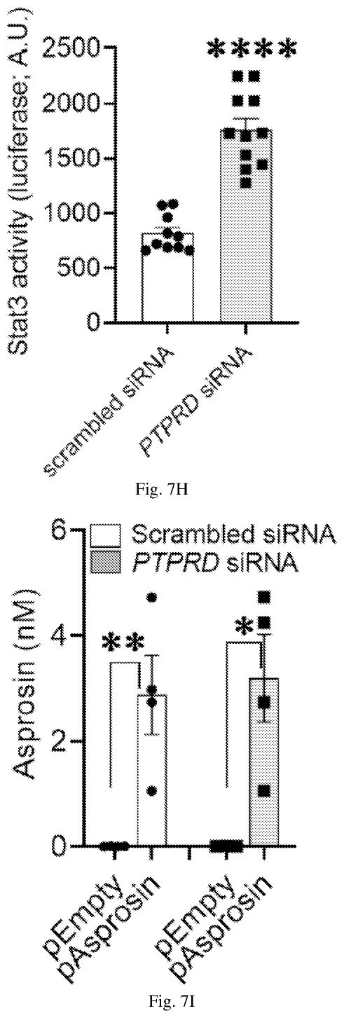

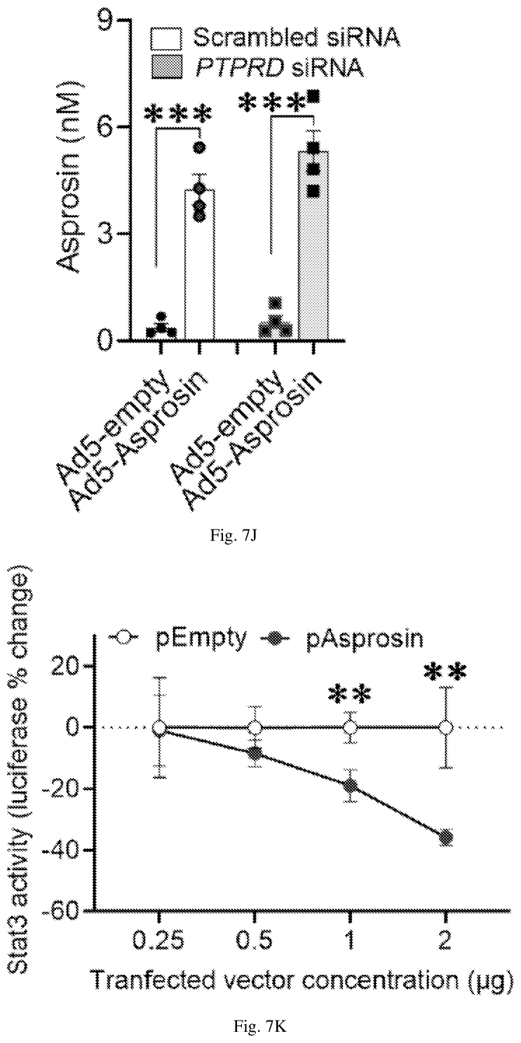

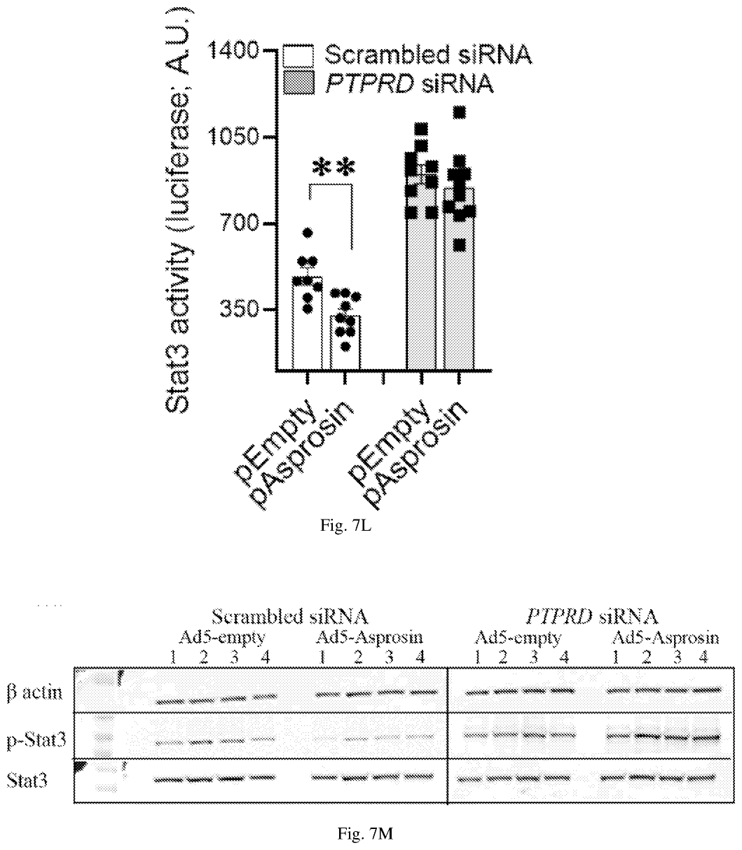

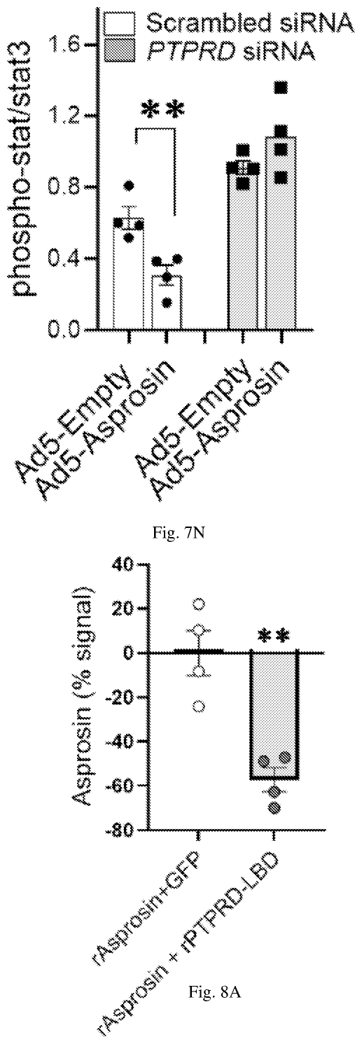

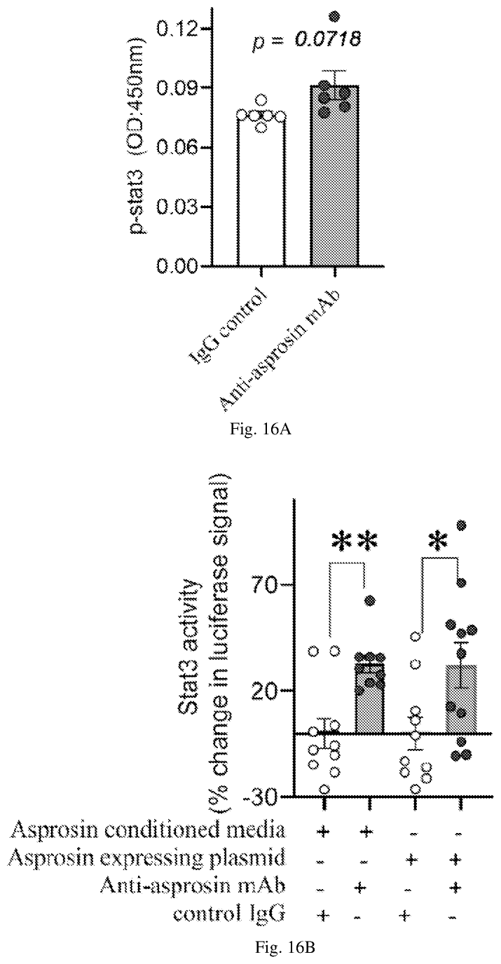

Ptprd phosphatase domain 1 (PD1) directly interacts with the transcription factor Stat3, leading to Stat3 dephosphorylation at Tyrosine residue 705 (p-Stat3) and inhibition of its transcriptional activity. This suggests that Stat3 phosphorylation and transcriptional activity can serve as a measure of asprosin-mediated Ptprd signaling. In support of this, we found p-Stat3 levels to be significantly lower in the hypothalamus of mice exposed to higher circulating asprosin via tail-vein injection with Ad5-FBN1 compared with mice that received Ad5-empty (FIG. 7A). Conversely, we observed significantly higher hypothalamic p-Stat3 levels in Ptprd^{-/-} mice when compared with WT (FIGS. 7B, D). Further, we measured overnight changes in hypothalamic p-Stat3 levels upon treatment with a single dose of the anti-asprosin mAb or IgG control in DIO mice without access to food. The lack of access to food through the length of the experiment ensured that the two groups (IgG versus anti-asprosin mAb) would have comparable adiposity. Higher hypothalamic p-Stat3 levels were observed in DIO mice subjected to asprosin neutralization (FIG. 16A), demonstrating that the effect of asprosin inhibition on hypothalamic p-Stat3 was free of potential confounding effects of change in adiposity or body weight.

To interrogate whether asprosin could activate endogenous Ptprd in a cell autonomous manner, we used HEK293T cells that naturally express PTPRD and STAT3. PTPRD knockdown using siRNA significantly increased p-Stat3 levels and Stat3 transcriptional activity (FIGS. 7E-H). Recombinant asprosin has been shown to display variable activity. Therefore, we transduced or transfected HEK293T cells with adenovirus or mammalian-expression plasmid encoding human cleaved asprosin (with an IL2 signal peptide to induce secretion) to enhance media asprosin levels (FIGS. 7I, J). This resulted in dose-dependent decline in Stat3 transcriptional activity (FIG. 7K). Additionally, we found that introduction of the anti-asprosin mAb in the media enhances Stat3 activity in cells subjected to asprosin overexpression or exposed to asprosin conditioned media compared with the control IgG (FIG. 16B), showing that extracellular asprosin neutralization completely prevents asprosin signaling independent of whether asprosin is overexpressed and secreted or delivered via conditioned media. Importantly, Ptprd knockdown rendered the cells unresponsive to asprosin-mediated suppression of p-Stat3 levels and Stat3 transcriptional activity (FIGS. 7L-N). Collectively, these results demonstrate cell-autonomous activation of Ptprd by asprosin.

Introduction of the PTPRD Ligand-Binding-Domain (PTPRD-LBD) into the Circulation Sequesters Asprosin to Decrease Appetite, Body Weight and Blood Glucose in Obese Mice

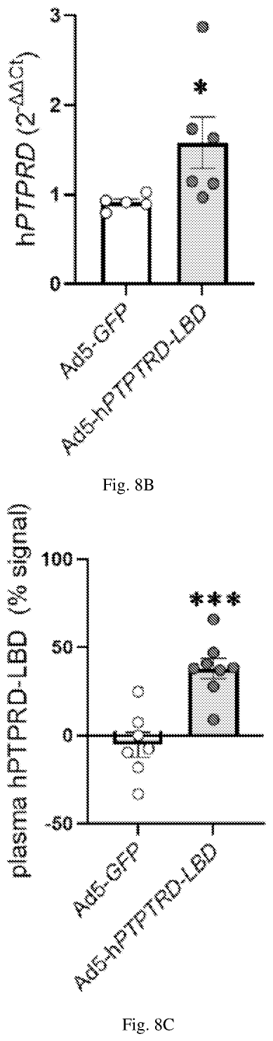

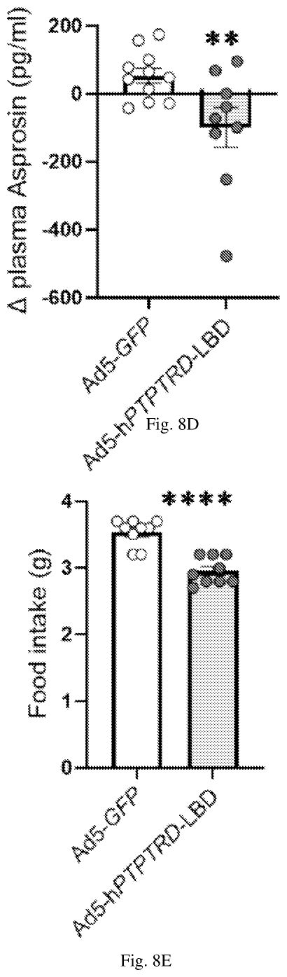

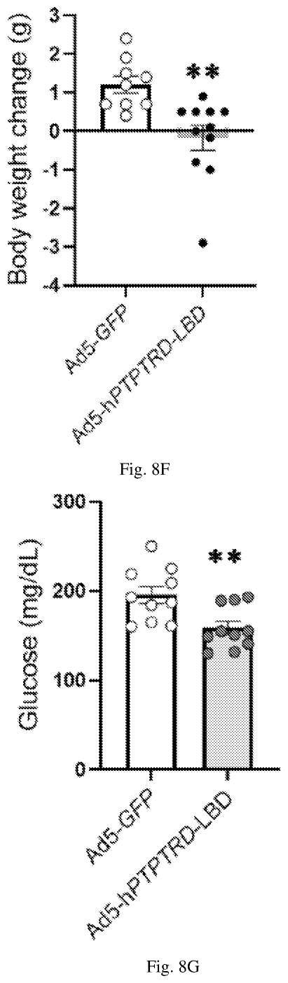

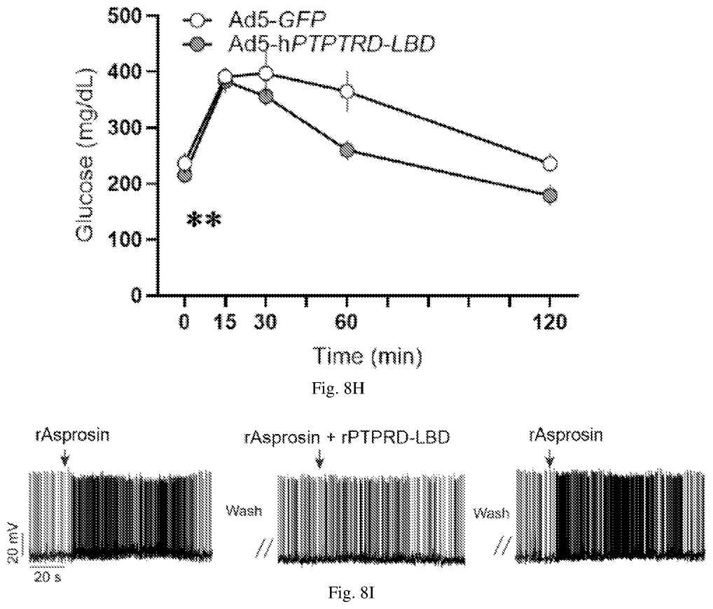

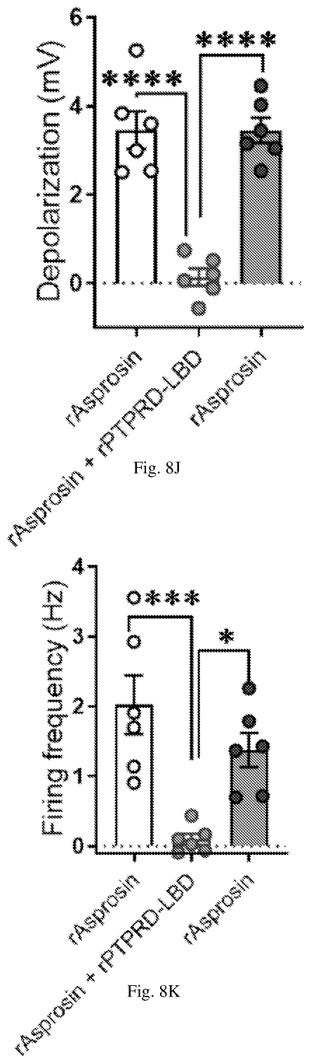

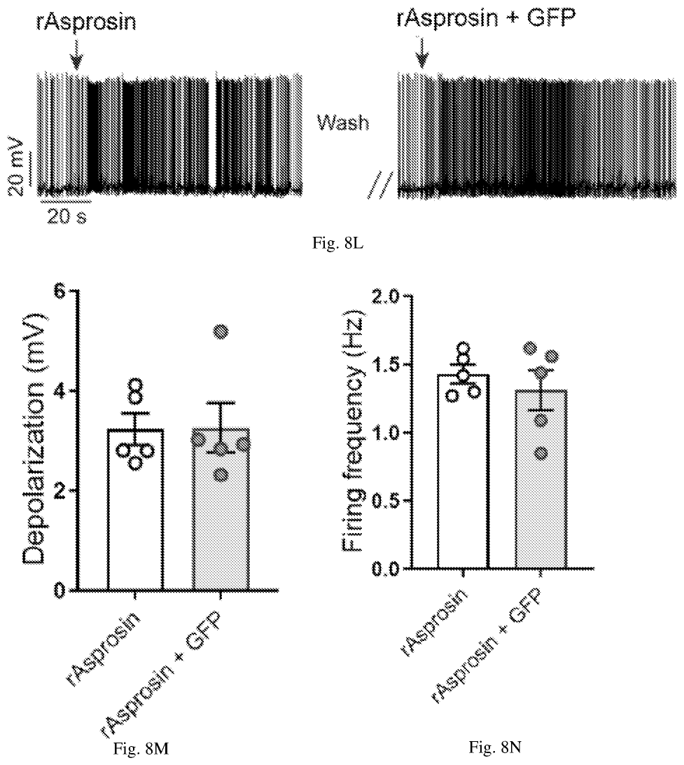

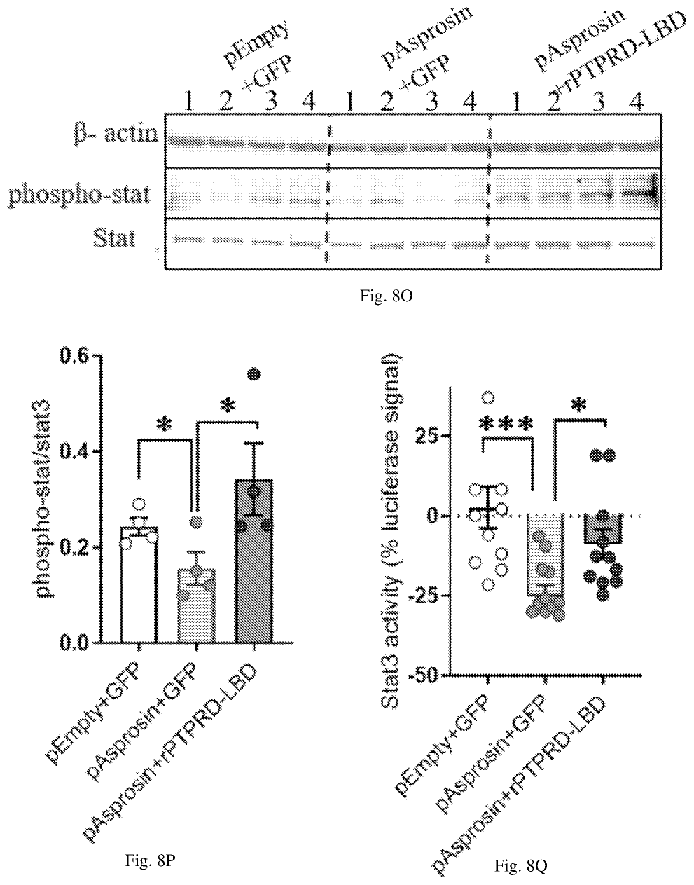

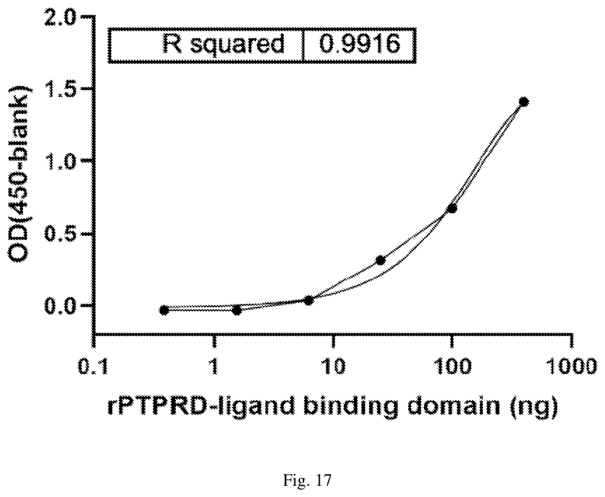

We have previously validated the use of asprosin-neutralizing monoclonal antibodies as a therapeutic against metabolic syndrome. Similar to the anti-asprosin mAb approach, here we tested whether asprosin can be sequestered, and metabolic syndrome ameliorated with introduction of the PTPRD ligand-binding-domain (PTPRD-LBD) in the circulation of mice with diet-induced obesity. The level of free asprosin detected by ELISA was significantly reduced when recombinant asprosin was incubated with recombinant PTPRD-LBD (FIG. 8A), indicating asprosin sequestration by the PTPRD-LBD. DIO mice were exposed to elevated plasma PTPRD-LBD by transducing mice with adenovirus (Ad5) encoding PTPRD-LBD (tagged with an IL2 signal peptide to induce secretion). We again chose the adenoviral strategy to avoid the unreliability of the recombinant protein approach in vivo. We developed a sandwich ELISA to detect his-tagged PTPRD-LBD in mouse plasma (FIG. 17) and demonstrated an elevation in the PTPRD-LBD levels in the plasma of DIO mice injected with Ad5-IL2-PTPRD-LBD concurrent with increased PTPRD-LBD expression in the liver (FIGS. 8B, C). Of note, DIO mice serve as a model of pathologically high plasma asprosin. We found significantly reduced levels of free asprosin in the plasma of DIO mice with elevated plasma PTPRD-LBD (FIG. 8D) indicating that plasma asprosin can be successfully sequestered by the circulating PTPRD-LBD. Introduction of the PTPRD-LBD in the plasma was associated with a significant reduction in daily food intake, body weight, blood glucose levels and glucose tolerance in DIO mice (FIGS. 8E-H). These results are virtually identical to the effects of anti-asprosin mAbs in DIO mice.

As a second line of evidence, we found that recombinant PTPRD-LBD, but not recombinant GFP, was able to completely prevent asprosin-mediated AgRP neuron activation in ex vivo hypothalamic slice electrophysiology (FIGS. 8I-N). Again, these results are virtually identical to the effects of anti-asprosin mAbs.

We extended these studies to an in vitro setup to interrogate whether introducing the PTPRD-LBD into the media could interfere with asprosin-mediated PTPRD signaling in a cell autonomous manner, as measured by p-Stat3 levels and Stat3 transcriptional activity. We transfected HEK293T cells with a plasmid encoding human cleaved asprosin (tagged with an IL2 signal peptide to induce secretion) and found that asprosin-mediated reduction in p-Stat3 levels and Stat3 transcriptional activity was abrogated upon introduction of recombinant PTPRD-LBD into the media (FIG. 8O-Q).

These in vivo, ex vivo and in vitro results suggest that circulating asprosin can be successfully inhibited via a “receptor-trap” approach using the PTPRD-LBD and may serve as a new therapeutic strategy in parallel to the anti-asprosin mAb approach to combat metabolic syndrome.

To achieve the goal of identifying the asprosin receptor that modulates its orexigenic and body weight maintaining functions, we performed unbiased screening of asprosin-interacting proteins in the mouse brain. Three independent screens identified protein tyrosine phosphatase receptor type δ (Ptprd) as an asprosin-interactor. This observation was confirmed biochemically, with single-to double-digit nanomolar affinity with multiple assays, well within the range traditionally displayed by hormones and their receptors. However, the absence of possible binding potentiators and/or the absence of the correct in vivo context from the purely in vitro MST/BLI/SPR techniques, could potentially underestimate the binding affinity.

Published AgRP neuron transcriptome analysis showed that Ptprd is highly expressed in AgRP neurons, the key cell type we have previously shown to respond to asprosin in a cell autonomous manner. Based on these results, we embarked on a multi-pronged experimental strategy to test the necessity of Ptprd for asprosin-mediated AgRP neuron activation and orexigenic function. Whether Ptprd is genetically ablated, or its ligand-binding domain (Ptprd-LBD) introduced into the circulation to sequester plasma asprosin, the result is a corresponding decrease in appetite and body weight. Marked leaness, reduced adiposity, hypophagia and a compensatory reduction in energy expenditure of Ptprd^{-/-} mice completely recapitulates the metabolic phenotype associated with genetic asprosin deficiency in humans (NPS patients) and mice (Fbnl^{NSP/+}).

Further, like Fbnl^{NPS/+} mice, mice with Ptprd ablated specifically in AgRP neurons, constitutively or in the adult setting, were protected from diet-induced-obesity. Importantly, deletion of Piprd from AgRP neurons rendered them unresponsive to exogenous asprosin. At the whole organism level, asprosin gain-of-function (via increase in plasma asprosin through an adenoviral-mediated approach) failed to increase appetite in Ptprd^{-/-} mice compared with WT, validating the absolute necessity of Ptprd for asprosin's orexigenic function. Interestingly, Ptprd loss is not associated with alterations in blood glucose or glucose tolerance, and asprosin gain-of-function in Ptprd^{-/-} mice results in changes in blood glucose and glucose tolerance comparable to WT mice, suggesting that Ptprd is necessary for only the orexigenic function of asprosin, and that the glucogenic effects of asprosin are mediated by a different receptor. This is consistent with Olfr734/OR4M1 serving as the hepatic (glucogenic) receptor for asprosin, and supported by the observed absence of the Ptprd transcript in the liver and that of the Olfr734 transcript in AgRP neurons.

Several clinical and pre-clinical studies have implicated PTPRD variants and SNPs as associative factors for metabolic disturbances, including type 2 diabetes, anorexia nervosa and anti-psychotic medication induced weight gain. We previously demonstrated the potent efficacy of anti-asprosin mAbs in multiple mouse models of metabolic syndrome. Given the association of asprosin deficiency with marked hypophagia, leanness, and protection from diet-induced metabolic syndrome; and its elevation in patients with metabolic syndrome; neutralization of asprosin or its downstream signaling can be an exceptionally promising therapeutic strategy for treatment of metabolic syndrome. The identification of Ptprd as an asprosin receptor provides a new modality for another clinical strategy for the pharmacological inhibition of the asprosin pathway in metabolic syndrome patients. As proof-of-concept, we demonstrate that the PTPRD ligand-binding domain (PTPRD-LBD) when introduced into the circulation of DIO mice leads to a reduction in appetite, body weight, and blood glucose; similar to that seen with use of anti-asprosin mAbs. Concordant with reduced appetite, asprosin-mediated AgRP neuron activation and Stat3-dephosphorylation/deactivation were both suppressed by the PTPRD-LBD. Interestingly, in contrast to Ptprd'-mice which display no alterations in glucose homeostasis, this “receptor-trap” strategy also improved hyperglycemia in DIO mice, indicating sequestration of asprosin from both its receptors (Ptprd and Olfr734). Thus, like anti-asprosin mAbs, use of the soluble PTPRD-LBD is a dual-effect therapy that targets the two spatio-temporally distinct functions of asprosin-orexigenic and glucogenic. This pharmacologic approach could serve as stand-alone or combination therapy with anti-asprosin mAbs and is reminiscent of the TNFα targeting approach against inflammatory diseases (Humira—anti-TNFα mAb, Enbrel-TNFα receptor trap). We envision further studies assessing the potential for off-target or toxic effects in the future as we contemplate a drug development path to the clinic. However, the discovery of Ptprd as an orexigenic asprosin-receptor, and demonstration of the PTPRD-LBD as a pharmacologic inhibitor of the asprosin pathway provides a new approach toward anti-metabolic syndrome pharmacotherapy.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1. A fusion polypeptide comprising a therapeutic peptide having an amino acid sequence substantially identical to an extracellular portion of the amino acid sequence of protein tyrosine phosphatase receptor type δ (PTPRD) that binds to asprosin and at least one polypeptide having an amino acid sequence heterologous to the therapeutic peptide.

2. The fusion polypeptide of claim 1, wherein the therapeutic peptide has an amino acid sequence at least about 70% identical to at least about 10 consecutive amino acids of SEQ ID NO: 2.

3. The fusion polypeptide of claim 1, wherein the therapeutic peptide has an amino acid sequence at least about 70% identical to SEQ ID NO: 2.

4. The fusion polypeptide of claim 1, wherein the therapeutic peptide has an amino acid sequence at least about 70% identical to at least about 10 consecutive amino acids of SEQ ID NO: 3.

5. The fusion polypeptide of claim 1 4, wherein the therapeutic peptide has a binding affinity K_{D} to asprosin less than about 10 μM.

6. The fusion polypeptide of claim 1, wherein the at least one heterologous polypeptide comprises an antibody or antigen binding fragment thereof, a glucagon-like peptide-1 receptor (GLP-1R) agonist, a Fc portion of an immunoglobulin, an albumin peptide, an albumin binding domain (ABD), a peptide linker, a signal peptide, or a combination thereof.

7. The fusion polypeptide of claim 1, wherein the heterologous polypeptide comprises a glucagon-like peptide-1 receptor (GLP-1R) agonist.

8. The fusion polypeptide of claim 7, wherein the heterologous polypeptide is selected from a bioactive GLP-1, a GLP-1 analogue, or a GLP-1 substitute.

9. The fusion polypeptide of claim 7, wherein the GLP-1R agonist is selected from GLP-1 (7-37), GLP-1 (7-36) amide, exendin-4, liraglutide, CJC-1131, albugon, albiglutide, exantide, exenatide-LAR, oxyntomodulin, lixisenatide, geniproside, or a peptide fragment thereof with GLP-1R agonistic activity.

10. The fusion polypeptide of claim 7, wherein the peptide linker links the therapeutic peptide to the GLP-1R agonist.

11. The fusion polypeptide of claim 10, wherein the linker comprises a protease cleavable site.

12. The fusion polypeptide of claim 11, wherein the linker includes Factor Xa cleavage site.

13. The fusion polypeptide of claim 10, wherein the protease cleavage site comprises the sequence IEGR (SEQ ID NO: 15) or GGGRR (SEQ ID NO: 16).

14. (canceled)

15. (canceled)

16. The fusion polypeptide of claim 6, wherein the signal peptide is an IL-2 signal peptide that promotes secretion of the fusion polypeptide from cells.

17-27. (canceled)

28. A method of inhibiting asprosin mediated orexigenesis and/or glucogenesis in a subject in need thereof, the method comprising:
administering to the subject a therapeutic peptide that includes an amino acid sequence substantially identical to an extracellular portion of the amino acid sequence of protein tyrosine phosphatase receptor type δ (PTPRD) that binds to asprosin.

29. The method of claim 28, wherein the therapeutic peptide has an amino acid sequence at least about 70 consecutive amino acids of SEQ ID NO: 2.

30. The method of claim 28, wherein the therapeutic peptide has an amino acid sequence at least about 70% identical to SEQ ID NO: 2.

31. The method of claim 28, wherein the therapeutic peptide has an amino acid sequence at least about 70% identical to at least about 10 consecutive amino acids of SEQ ID NO: 3.

32. The method of any of claim 28, wherein the therapeutic peptide has a binding affinity K_{D} to asprosin less than about 10 μM.

33. The method of claim 28, wherein the therapeutic peptide is linked to a heterologous polypeptide.

34-67. (canceled)
